(19) United States
(12) Patent Application Publication
(10) Pub. No.: US 2016/0115490 A1
(43) Pub. Date: Apr. 28, 2016

(54) Bacterial Mutants with Improved Transformation Efficiency

(71) Applicant: Novozymes A/S, Bagsvaerd (DK)

(72) Inventors: Barbara Cherry, Davis, CA (US); Randy Berka, Davis, CA (US)

(73) Assignee: Novozymes A/S, Bagsvaerd (DK)

(21) Appl. No.: 14/890,023

(22) PCT Filed: Jun. 18, 2014
(86) PCT No.: PCT/US14/42977

§ 371 Date: Nov. 9, 2015

Related U.S. Application Data
(60) Provisional application No. 61/836,448, filed on Jun. 18, 2013.

(51) Int. Cl. C12N 15/75; C12P 1/04; C12P 21/00

(52) U.S. Cl. CPC C12N 15/75; C12P 21/00; C12P 1/04

(57) ABSTRACT

Provided herein are Bacillus mutants having improved transformation efficiency, comprising a disruption of an endogenous epsA-O operon. Also described are methods for producing the mutants, methods for generating transformants using the mutants, and methods for producing a polypeptide or fermentation product using the mutants.

### REFERENCE TO A SEQUENCE LISTING

This application contains a Sequence Listing in computer readable form, which is incorporated herein by reference.

### BACKGROUND

Genetic competence is a physiological state in which exogenous DNA can be internalized, leading to a transformation event (Berka et al., 2002, Mol. Microbiol. 43: 1331-1345), but is distinct from artificial transformation involving electroporation, protoplasts, and heat shock or CaCl₂ treatment. Natural competence has been observed in both Gram positive and Gram negative bacterial species (Dubnau, 1999, Annual Rev. Microbiol. 53: 217-244), and the process requires more than a dozen proteins whose expression is precisely choreographed to the needs of each organism.

Several hypotheses have been proposed regarding the purpose of natural competence, and they can be summarized as DNA for food, DNA for repair, and DNA for genetic diversity (Dubnau, 1999, supra). The DNA for food hypothesis is supported by observations that competence is a stationary phase phenomenon that occurs when cells are nutrient limited, and often a powerful nonspecific nuclease is co-expressed with transformation specific proteins. Evidence for the second hypothesis comes from the fact that genes encoding DNA repair enzymes are coordinately expressed with those encoding DNA transport proteins. Lastly, the DNA for genetic diversity hypothesis proposes that competence is a mechanism for exploring the fitness landscape via horizontal gene transfer. Observations that competence is regulated by a quorum-sensing mechanism and that it is a bistable condition (Avery, 2005, Trends Microbiol. 13: 459-462) support this hypothesis.

Public databases now contain a multitude of complete bacterial genomes, including several genomes from different strains of the same species. Recent analyses have shown, using pairwise whole genome alignments, that different strains of the same species may vary substantially in gene content. For example, genome comparisons of Escherichia coli strains CFT073, EDL933, and MG1655 revealed that only 39.2% of their combined set of proteins (gene products) are common to all three strains, highlighting the astonishing diversity among strains of the same species (Blattner et al., 1997, Science 277: 1453-1474; Hayashi et al., 2006, Mol. Syst. Biol. doi:10.1038:msb4100049; Perna et al., 2001, Nature 409: 529-533; Welch et al., 2002, Proc. Natl. Acad Sci. USA 99: 17020-17024). Furthermore, the genome sequence of E. coli strain CFT073 revealed 1,623 strain-specific genes (21.2%). From comparisons of this type, it is clearly seen that bacterial genomes are segmented into a common conserved backbone and strain-specific sequences. Typically the genome of a given strain within a species shows a mosaic structure in terms of the distribution of conserved “backbone” genes conserved among all strains and non-conserved genes that may have been acquired by horizontal transfer (Brzuszkiewicz et al., 2006, Proc. Natl. Acad. Sci. USA 103: 12879-12884; Welch et al., 2002, supra).

In terms of practical utility, transformation via natural competence is an extremely useful tool for constructing bacterial strains, e.g., Bacillus, which may contain altered alleles for chromosomal genes or plasmids assembled via recombinant DNA methods. Although transformation of certain species with plasmids and chromosomal DNA may be achieved via artificial means as noted above (e.g., electroporation, protoplasts, and heat shock or CaCl₂ treatment), introduction of DNA by natural competence offers clear advantages of simplicity, convenience, speed, and efficiency.

In Bacillus subtilis, only 5-10% of the cells in a population differentiate to a competent state (termed the K-state) via a process that involves quorum-sensing, signal transduction, and a cascade of gene expression (Avery, 2005, supra). At least 50 genes are known to be involved directly in competence, and as many as 165 genes are regulated (directly or indirectly) by the central transcription factor ComK (Berka et al., 2002, supra). The competence cascade in Bacillus subtilis consists of two regulatory modules punctuated by a molecular switch (FIG. 1) that involves ComS binding to the adaptor molecule MecA, thereby interfering with degradation of the transcription factor ComK by the ClpC/ClpP protease (Turgay et al., 1998, EMBO J. 17: 6730-6738).

In Bacillus subtilis, mecA inactivation has been shown to moderately elevate transformation efficiency due to increased availability of ComK (Hahn et al., 1995, Mol. Microbiol. 18: 755-767). A recent report suggests a Bacillus subtilis mecA deletion results in an increased expression of the eps and tasA operons (Prepiak et al., 2011, Mol. Microbiol., 80: 1014-1030) in accordance with the regulatory relationship between mecA and the eps and tasA regulons shown in FIG. 2.

With the exception of comP and comS, Bacillus licheniformis harbors orthologues of the genes necessary to achieve natural competence. Ostensibly naturally competent Bacillus licheniformis cells cannot be obtained due to the lack of a functional comS gene resulting in ComK continually sequestered by MecA, and proteolytically degraded by ClpC/P/MecA complex. The Applicant has shown that expression of ComS and ComK in Bacillus licheniformis can improve competence (US 2010/0028944).

Myxococcus xanthus mutants deficient in exopolysaccharide synthesis were reported to have increased transformation efficiency (Wang et al., 2011, Journal of Bacteriology 193: 2122-2132). However, Streptococcus gordonii mutants defective in extracellular polysaccharide production showed reduced competence (Zheng et al., 2011, Molecular Oral Microbiology 27: 83-94). Thus, the effect of exopolysaccharide appears to vary depending on host. The extent to which the exopolymeric substance (EPS) alters transformation competence in Bacillus cells has remained largely unknown.

Since Bacillus species provide a key platform for a variety of industrially relevant processes such as metabolic engineering and biochemical production, engineering strains that manifest improved competence is highly desirable for construction of new and improved production strains. The availability of a turn-key method for improving competence in Bacillus strains would improve the speed and efficiency with which chromosomal markers/alleles and expression vectors could be introduced. The present invention fulfills these and other needs.

### SUMMARY

Described herein are Bacillus mutants having improved transformation efficiency. Applicants have surprisingly found that disruption of the endogenous epsA-O operon in a Bacillus host cell shows significantly improved transformation efficiency compared to the wild-type parent.

One aspect is an isolated mutant of a parent Bacillus strain, comprising disruption of an endogenous epsA-O operon, wherein the mutant has improved transformation efficiency compared to the parent Bacillus strain that lacks disruption of the endogenous epsA-O operon, when cultivated under identical conditions. In some aspects, the Bacillus mutant is a Bacillus amyloliquefaciens mutant, a Bacillus licheniformis mutant or a Bacillus subtilis mutant.

Also described are methods for obtaining the Bacillus mutants, comprising disrupting an endogenous epsA-O operon in a parent Bacillus strain.

Also described are methods for obtaining a Bacillus transformant, comprising transforming a heterologous polynucleotide into a Bacillus mutant, wherein the mutant comprises a disruption of an endogenous epsA-O operon.

Also described are methods of producing a polypeptide, comprising: (a) cultivating a Bacillus transformant described herein comprising a heterologous polynucleotide encoding the polypeptide; and (b) recovering the polypeptide.

Also described are methods of producing a fermentation product, comprising: (a) cultivating the Bacillus transformant described herein comprising a heterologous polynucleotide encoding a polypeptide of the fermentation pathway; and (b) recovering the fermentation product.

### BRIEF DESCRIPTION OF THE FIGURES

FIG. 1 shows the competence regulatory cascade of Bacillus subtilis. Module 1 involves detection of the competence pheromone CSF and signal transduction via a phosphorelay mechanism resulting in synthesis of the ComS peptide. ComS interferes with proteolytic degradation of the transcription factor ComK via binding to MecA that activates Module 2 encoding the late competence functions encoding DNA transport machinery.

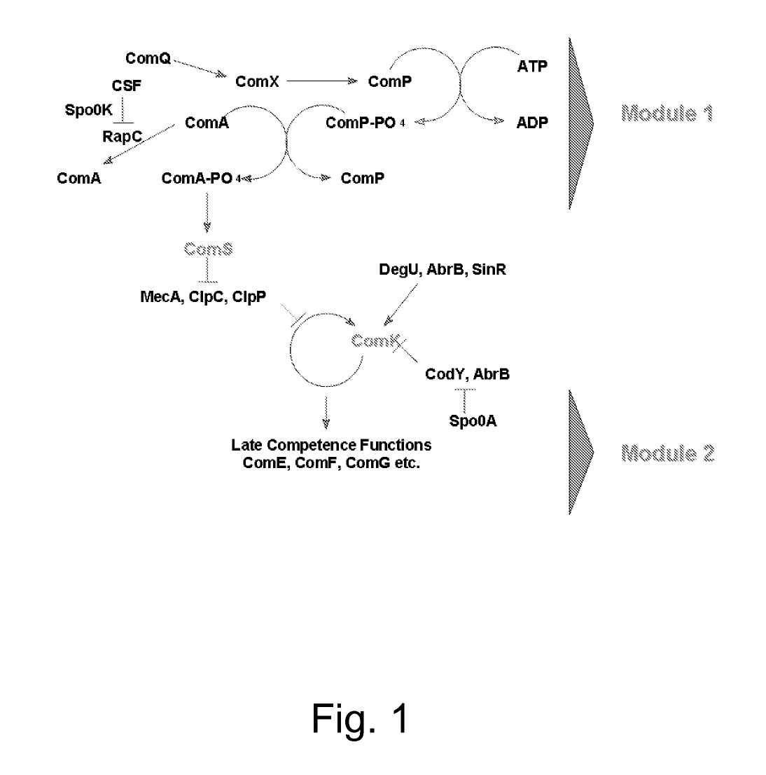

FIG. 2 shows the regulatory relationship between mecA and the eps and tasA regulons in Bacillus subtilis.

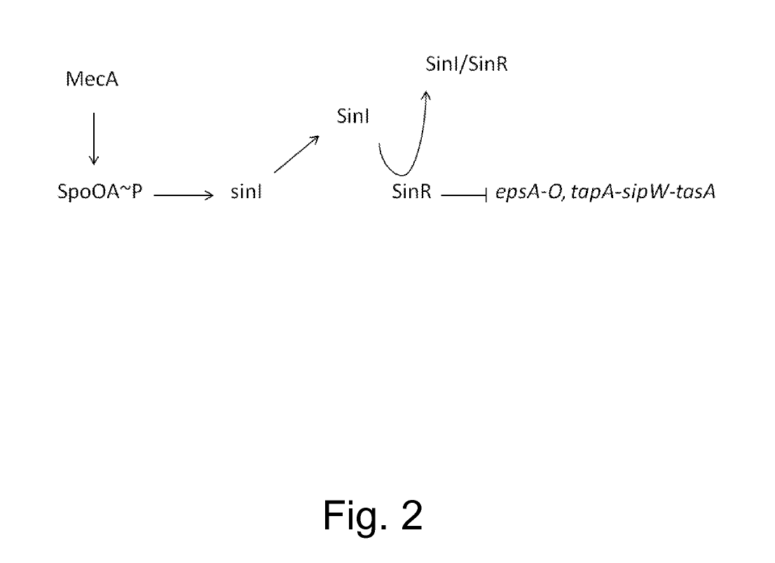

FIG. 3 shows the relative transformation efficiency of Bacillus strain BaC0171 comprising a disruption of the epsA-O operon compared to the non-disrupted parent strain using two micrograms of transforming plasmid DNA.

FIG. 4 shows the relative transformation efficiency of Bacillus strain BaC0171 comprising a disruption of the epsA-O operon compared to the non-disrupted parent strain using two micrograms of transforming plasmid DNA.

FIG. 5 shows the transformation efficiency of Bacillus strain TaHy9 comprising a mecA gene disruption.

### DEFINITIONS

Coding sequence: The term “coding sequence” means a polynucleotide sequence, which specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a sequence of genomic DNA, cDNA, a synthetic polynucleotide, and/or a recombinant polynucleotide.

Disruption: The term “disruption” means that a coding region and/or control sequence of a referenced gene is partially or entirely modified (such as by deletion, insertion, and/or substitution of one or more nucleotides) resulting in the absence (inactivation) or decrease in expression, and/or the absence or decrease of enzyme activity of the encoded polypeptide. The effects of disruption can be measured using techniques known in the art such as detecting the absence or decrease of enzyme activity using cell-free extract measurements referenced herein; or by the absence or decrease of corresponding mRNA (e.g., at least 25% decrease, at least 50% decrease, at least 60% decrease, at least 70% decrease, at least 80% decrease, or at least 90% decrease); the absence or decrease in the amount of corresponding polypeptide having enzyme activity (e.g., at least 25% decrease, at least 50% decrease, at least 60% decrease, at least 70% decrease, at least 80% decrease, or at least 90% decrease); or the absence or decrease of the specific activity of the corresponding polypeptide having enzyme activity (e.g., at least 25% decrease, at least 50% decrease, at least 60% decrease, at least 70% decrease, at least 80% decrease, or at least 90% decrease). Disruptions of a particular gene of interest can be generated by methods known in the art, e.g., by directed homologous recombination (see Methods in Yeast Genetics (1997 edition), Adams, Gottschling, Kaiser, and Stems, Cold Spring Harbor Press (1998)). Techniques to disrupt Bacillus genes are described herein and have been demonstrated in the art (see Stahl & Ferrari, 1984, J. Bacteriol. 158: 411-418).

epsA-O operon: The term “epsA-O operon” means a fifteen-gene operon known in Bacillus cells to be involved in exopolymeric substance (EPS) biosynthesis, modification, and export (Branda et al., 2001, Proc. Natl. Acad. Sci. USA 98: 11621-11626). The epsA-O operon, also designated as ybeK-T yvfA-F, is under control of both Spo0A and σ^{H}.

The term “disruption of an endogenous epsA-O operon” means a disruption resulting in the absence or decrease in expression of at least one coding sequence of the epsA-O operon, and/or the absence or decrease of enzyme activity of at least one encoded polypeptide of the epsA-O operon. Non-limiting examples of a disruption of an endogenous epsA-O operon include disruption of an operon promoter, and/or disruption of one or more (e.g., two, three, four, five, six, etc.) of the epsA-O operon coding sequences.

Improved transformation efficiency: The term “improved transformation efficiency” means that the referenced Bacillus mutant strain is capable of generating an increased number of transformants compared to the parent Bacillus strain when transformed and cultivated under identical conditions. Improved transformation efficiency can be demonstrated by generating an increased number of transformants, e.g., using transformation methods described in the Examples below. Improved transformation efficiency may also be demonstrated using methods previously described (e.g., Anagnostopoulos and Spizizen, 1961, J. Bacteriol. 81: 741-746). In some aspects, the Bacillus mutant strain is capable of producing at least 2-fold, e.g., at least 5-fold, at least 10-fold, at least 20-fold, at least 50-fold, at least 100-fold, at least 200-fold, at least 500-fold, at least 1000-fold, at least 2000-fold, at least 5000-fold, at least 10000-fold, at least 20000-fold, at least 50000-fold, or at least 100000-fold more transformants compared to the parent Bacillus strain, when cultivated under identical conditions.

Mutant: The term “mutant” means the resulting Bacillus strain after one or more disruptions are made to a parent Bacillus strain.

Parent: The term “parent” or “parent Bacillus strain” means a Bacillus strain to which a disruption is made to produce a mutant Bacillus strain described herein. The parent may be a naturally occurring (wild-type) or previously modified Bacillus strain.

Sequence Identity: The relatedness between two amino acid sequences or between two nucleotide sequences is described by the parameter “sequence identity”.

For purposes described herein, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes described herein, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Hybridization conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Heterologous polynucleotide: The term “heterologous polynucleotide” is defined herein as a polynucleotide that is not native to the host cell; a native polynucleotide in which one or more (e.g., two, several) structural modifications have been made to the coding region; a native polynucleotide whose expression is quantitatively altered as a result of manipulation of the DNA by recombinant DNA techniques, e.g., a different (foreign) promoter linked to the polynucleotide; or a native polynucleotide whose expression is quantitatively altered by the introduction of one or more extra copies of the polynucleotide into the host cell.

Isolated: The term “isolated” means a substance in a form or environment that does not occur in nature. Non-limiting examples of isolated substances include (1) any non-naturally occurring substance, (2) any substance including, but not limited to, any host cell, enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated.

Endogenous: With reference to a gene/operon/coding sequence, the term “endogenous” means the referenced polynucleotide native to a parent Bacillus strain prior to any disruption.

Nucleic acid construct: The term “nucleic acid construct” means a polynucleotide comprises one or more (e.g., two, several) control sequences. The polynucleotide may be single-stranded or double-stranded, and may be isolated from a naturally occurring gene, modified to contain segments of nucleic acids in a manner that would not otherwise exist in nature, or synthetic.

Control sequence: The term “control sequence” means a nucleic acid sequence necessary for polypeptide expression. Control sequences may be native or foreign to the polynucleotide encoding the polypeptide, and native or foreign to each other. Such control sequences include, but are not limited to, a leader sequence, polyadenylation sequence, propeptide sequence, promoter sequence, signal peptide sequence, and transcription terminator sequence. The control sequences may be provided with linkers for the purpose of introducing specific restriction sites facilitating ligation of the control sequences with the coding region of the polynucleotide encoding a polypeptide.

Operably linked: The term “operably linked” means a configuration in which a control sequence is placed at an appropriate position relative to the coding sequence of a polynucleotide such that the control sequence directs the expression of the coding sequence.

Expression: The term “expression” includes any step involved in the production of the polypeptide including, but not limited to, transcription, post-transcriptional modification, translation, post-translational modification, and secretion. Expression can be measured—for example, to detect increased expression—by techniques known in the art, such as measuring levels of mRNA and/or translated polypeptide.

Expression vector: The term “expression vector” means a linear or circular DNA molecule that comprises a polynucleotide encoding a polypeptide and is operably linked to control sequences, wherein the control sequences provide for expression of the polynucleotide encoding the polypeptide. At a minimum, the expression vector comprises a promoter sequence, and transcriptional and translational stop signal sequences.

Host cell: The term “host cell” means any cell type that is susceptible to transformation, transfection, transduction, and the like with a nucleic acid construct or expression vector. The term “host cell” encompasses any progeny of a parent cell that is not identical to the parent cell due to mutations that occur during replication.

Transformation: The term “transformation” means introducing a heterologous polynucleotide into a Bacillus cell so that the DNA is maintained as a chromosomal integrant or as a self-replicating extra-chromosomal vector. The resulting Bacillus cell following transformation is described herein as a “transformant.”

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Reference to “about” a value or parameter herein includes aspects that are directed to that value or parameter per se. For example, description referring to “about X” includes the aspect “X”. When used in combination with measured values, “about” includes a range that encompasses at least the uncertainty associated with the method of measuring the particular value, and can include a range of plus or minus two standard deviations around the stated value.

As used herein and in the appended claims, the singular forms “a,” “or,” and “the” include plural referents unless the context clearly dictates otherwise. It is understood that the aspects described herein include “consisting” and/or “consisting essentially of” aspects.

Unless defined otherwise or clearly indicated by context, all technical and scientific terms used herein have the same meaning as commonly understood by one of ordinary skill in the art.

### DETAILED DESCRIPTION

### Bacillus Mutants

Described herein, inter alia, are isolated mutants of a parent Bacillus strain, comprising a disruption of an endogenous epsA-O operon which provides improved transformation efficiency. Without being bound by theory, targeted disruption of the epsA-O operon can decrease the production of an exopolymeric substance (EPS)—a physical barrier that appears to be inhibiting transformation efficiency in Bacilli.

The parent strain of the mutants and related methods may be any Bacillus strain, such as a wild-type Bacillus or a mutant thereof. In some aspects, the parent Bacillus strain is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis strain. In some aspects, the Bacillus mutants are selected from Bacillus amyloliquefaciens, Bacillus licheniformis, and Bacillus subtilis.

The disrupted epsA-O operon may be any suitable endogenous Bacillus epsA-O operon, e.g., an endogenous epsA-O operon that comprises one or more (e.g., two, several) of the coding sequences of SEQ ID NOs: 1-45 shown in Table 1, which encode for the corresponding polypeptides of SEQ ID NOs: 46-90 shown in Table 2.

TABLE 1

| | Bacillus | Bacillus | Bacillus |
|---|---|---|---|
| | amyloliquefaciens | licheniformis | subtilis |
| epsA | SEQ ID NO: 1 | SEQ ID NO: 2 | SEQ ID NO: 3 |
| epsB | SEQ ID NO: 4 | SEQ ID NO: 5 | SEQ ID NO: 6 |
| epsC | SEQ ID NO: 7 | SEQ ID NO: 8 | SEQ ID NO: 9 |
| epsD | SEQ ID NO: 10 | SEQ ID NO: 11 | SEQ ID NO: 12 |
| epsE | SEQ ID NO: 13 | SEQ ID NO: 14 | SEQ ID NO: 15 |
| epsF | SEQ ID NO: 16 | SEQ ID NO: 17 | SEQ ID NO: 18 |
| epsG | SEQ ID NO: 19 | SEQ ID NO: 20 | SEQ ID NO: 21 |
| epsH | SEQ ID NO: 22 | SEQ ID NO: 23 | SEQ ID NO: 24 |
| epsI | SEQ ID NO: 25 | SEQ ID NO: 26 | SEQ ID NO: 27 |
| epsJ | SEQ ID NO: 28 | SEQ ID NO: 29 | SEQ ID NO: 30 |
| epsK | SEQ ID NO: 31 | SEQ ID NO: 32 | SEQ ID NO: 33 |
| epsL | SEQ ID NO: 34 | SEQ ID NO: 35 | SEQ ID NO: 36 |
| epsM | SEQ ID NO: 37 | SEQ ID NO: 38 | SEQ ID NO: 39 |
| epsN | SEQ ID NO: 40 | SEQ ID NO: 41 | SEQ ID NO: 42 |
| epsO | SEQ ID NO: 43 | SEQ ID NO: 44 | SEQ ID NO: 45 |

TABLE 2

| | Bacillus | Bacillus | Bacillus |
|---|---|---|---|
| | amyloliquefaciens | licheniformis | subtilis |
| EPSA | SEQ ID NO: 46 | SEQ ID NO: 47 | SEQ ID NO: 48 |
| EPSB | SEQ ID NO: 49 | SEQ ID NO: 50 | SEQ ID NO: 51 |
| EPSC | SEQ ID NO: 52 | SEQ ID NO: 53 | SEQ ID NO: 54 |
| EPSD | SEQ ID NO: 55 | SEQ ID NO: 56 | SEQ ID NO: 57 |
| EPSE | SEQ ID NO: 58 | SEQ ID NO: 59 | SEQ ID NO: 60 |
| EPSF | SEQ ID NO: 61 | SEQ ID NO: 62 | SEQ ID NO: 63 |
| EPSG | SEQ ID NO: 64 | SEQ ID NO: 65 | SEQ ID NO: 66 |
| EPSH | SEQ ID NO: 67 | SEQ ID NO: 68 | SEQ ID NO: 69 |
| EPSI | SEQ ID NO: 70 | SEQ ID NO: 71 | SEQ ID NO: 72 |
| EPSJ | SEQ ID NO: 73 | SEQ ID NO: 74 | SEQ ID NO: 75 |
| EPSK | SEQ ID NO: 76 | SEQ ID NO: 77 | SEQ ID NO: 78 |
| EPSL | SEQ ID NO: 79 | SEQ ID NO: 80 | SEQ ID NO: 81 |
| EPSM | SEQ ID NO: 82 | SEQ ID NO: 83 | SEQ ID NO: 84 |
| EPSN | SEQ ID NO: 85 | SEQ ID NO: 86 | SEQ ID NO: 87 |
| EPSO | SEQ ID NO: 88 | SEQ ID NO: 89 | SEQ ID NO: 90 |

In one aspect, the endogenous epsA-O operon of the isolated mutant Bacillus strain (a) encodes for at least one polypeptide having at least 60%, e.g., at least 70%, at least 75%, at least 80%, at least 85%, at least 90%, at least 95%, at least 96%, at least 97%, at least 98%, at least 99%, or 100% sequence identity to any of SEQ ID NOs: 46-90; (b) comprises at least one coding sequence that hybridizes under at least low, medium, medium-high, high, or very high stringency conditions with the full-length complementary strand of any of SEQ ID NOs: 1-45; or (c) comprises at least one coding sequence that has at least 60%, e.g., at least 70%, at least 75%, at least 80%, at least 85%, at least 90%, at least 95%, at least 96%, at least 97%, at least 98%, at least 99%, or 100% sequence identity to any of SEQ ID NOs: 1-45.

In some aspects, the endogenous epsA-O operon encodes for at least one polypeptide having at least 60%, e.g., at least 70%, at least 75%, at least 80%, at least 85%, at least 90%, at least 95%, at least 96%, at least 97%, at least 98%, at least 99%, or 100% sequence identity to any of SEQ ID NOs: 46-90. In some aspects, the endogenous epsA-O operon encodes for at least one polypeptide having a sequence that differs by no more than ten amino acids, e.g., by no more than five amino acids, by no more than four amino acids, by no more than three amino acids, by no more than two amino acids, or by one amino acid from any of SEQ ID NOs: 46-90. In some aspects, the endogenous epsA-O operon encodes for at least one polypeptide comprising or consisting of any of SEQ ID NOs: 46-90.

In some aspects, the endogenous epsA-O operon comprises at least one coding sequence that has at least 60%, e.g., at least 70%, at least 75%, at least 80%, at least 85%, at least 90%, at least 95%, at least 96%, at least 97%, at least 98%, at least 99%, or 100% sequence identity to any of SEQ ID NOs: 1-45. In some aspects, the endogenous epsA-O operon comprises at least one coding sequence comprising or consisting of any of SEQ ID NOs: 1-45.

In some aspects, the endogenous epsA-O operon comprises at least one coding sequence that hybridizes under at least low, medium, medium-high, high, or very high stringency conditions with the full-length complementary strand of any of SEQ ID NOs: 1-45.

Disruption of the endogenous epsA-O operon may occur to a control sequence (e.g., promoter) and/or one or more (e.g., two, several) of the epsA, epsB, epsC, epsD, epsE, epsF, epsG, epsH, epsI, epsJ, epsK, epsL, epsM, epsN, or epsO coding sequences.

In one aspect, the isolated mutant Bacillus strain comprises a disruption of the promoter sequence of the endogenous epsA-O operon.

In some aspects, disruption of the endogenous epsA-O operon comprises disruption of at least two (e.g., at least three, four, five, six, etc.) of the epsA-O operon coding sequences.

In some aspects, the mutants have improved transformation efficiency compared to the parent Bacillus strain that lacks disruption of the endogenous epsA-O operon, when cultivated under identical conditions. In some embodiments, the mutants are capable of producing at least 10-fold (e.g., at least 100-fold, at least 1000-fold, at least 10000-fold, or at least 100000-fold) more transformants compared to the parent Bacillus strain that lacks disruption of the endogenous epsA-O operon, when cultivated under identical conditions.

epsA

In some aspects, disruption of the endogenous epsA-O operon occurs in an endogenous epsA coding sequence. In one aspect, the endogenous epsA coding sequence is inactivated.

Examples of endogenous Bacillus epsA coding sequences include the Bacillus amyloliquefaciens epsA coding sequence of SEQ ID NO: 1 (which encodes the polypeptide of SEQ ID NO: 46), the Bacillus licheniformis epsA coding sequence of SEQ ID NO: 2 (which encodes the polypeptide of SEQ ID NO: 47), and the Bacillus subtilis epsA coding sequence of SEQ ID NO: 3 (which encodes the polypeptide of SEQ ID NO: 48).

In some embodiments, the endogenous epsA coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 46, 47, or 48; (b) hybridizes under at least low, medium, medium-high, high, or very high stringency conditions with the full-length complementary strand of SEQ ID NO: 1, 2, or 3; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 1, 2, or 3.

In some embodiments, the endogenous epsA coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 46, 47, or 48. In some embodiments, the endogenous epsA coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 46, 47, or 48. In some embodiments, the endogenous epsA coding sequence encodes for a polypeptide comprising or consisting of SEQ ID NO: 46, 47, or 48.

In some embodiments, the endogenous epsA coding sequence hybridizes under at least low, medium, medium-high, high, or very high stringency conditions with the full-length complementary strand of SEQ ID NO: 1, 2, or 3.

In some embodiments, the endogenous epsA coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 1, 2, or 3. In some embodiments, the endogenous epsA coding sequence comprises or consists of SEQ ID NO: 1, 2, or 3.

In some aspects, the mutant produces at least 25% less (e.g., at least 50% less, at least 60% less, at least 70% less, at least 80% less, at least 90% less, or 100% less) of the polypeptide encoded by the endogenous epsA coding sequence compared to the parent Bacillus strain that lacks disruption of the endogenous epsA-O operon, when cultivated under identical conditions.

epsB

In some aspects, disruption of the endogenous epsA-O operon occurs in an epsB coding sequence. In one aspect, the endogenous epsB coding sequence is inactivated.

Examples of endogenous Bacillus epsB coding sequences include the Bacillus amyloliquefaciens epsB coding sequence of SEQ ID NO: 4 (which encodes the polypeptide of SEQ ID NO: 49), the Bacillus licheniformis epsB coding sequence of SEQ ID NO: 5 (which encodes the polypeptide of SEQ ID NO: 50), and the Bacillus subtilis epsB coding sequence of SEQ ID NO: 6 (which encodes the polypeptide of SEQ ID NO: 51).

In some embodiments, the endogenous epsB coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 49, 50, or 51; (b) hybridizes under at least low, medium, medium-high, high, or very high stringency conditions with the full-length complementary strand of SEQ ID NO: 4, 5, or 6; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 4, 5, or 6.

In some embodiments, the endogenous epsB coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 49, 50, or 51. In some embodiments, the endogenous epsB coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 49, 50, or 51. In some embodiments, the endogenous epsB coding sequence encodes for a polypeptide comprising or consisting of SEQ ID NO: 49, 50, or 51.

In some embodiments, the endogenous epsB coding sequence hybridizes under at least low, medium, medium-high, high, or very high stringency conditions with the full-length complementary strand of SEQ ID NO: 4, 5, or 6.

In some embodiments, the endogenous epsB coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 4, 5, or 6. In some embodiments, the endogenous epsB coding sequence comprises or consists of SEQ ID NO: 4, 5, or 6.

In some aspects, the mutant produces at least 25% less (e.g., at least 50% less, at least 60% less, at least 70% less, at least 80% less, at least 90% less, or 100% less) of the polypeptide encoded by the endogenous epsB coding sequence compared to the parent Bacillus strain that lacks disruption of the endogenous epsA-O operon, when cultivated under identical conditions.

epsC

In some aspects, disruption of the endogenous epsA-O operon occurs in an endogenous epsC coding sequence. In one aspect, the endogenous epsC coding sequence is inactivated.

Examples of endogenous Bacillus epsC coding sequences include the Bacillus amyloliquefaciens epsC coding sequence of SEQ ID NO: 7 (which encodes the polypeptide of SEQ ID NO: 52), the Bacillus licheniformis epsC coding sequence of SEQ ID NO: 8 (which encodes the polypeptide of SEQ ID NO: 53), and the Bacillus subtilis epsC coding sequence of SEQ ID NO: 9 (which encodes the polypeptide of SEQ ID NO: 54).

In some embodiments, the endogenous epsC coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 52, 53, or 54; (b) hybridizes under at least low, medium, medium-high, high, or very high stringency conditions with the full-length complementary strand of SEQ ID NO: 7, 8, or 9; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 7, 8, or 9.

In some embodiments, the endogenous epsC coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 52, 53, or 54. In some embodiments, the endogenous epsC coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 52, 53, or 54. In some embodiments, the endogenous epsC coding sequence that encodes for a polypeptide comprising or consisting of SEQ ID NO: 52, 53, or 54.

In some embodiments, the endogenous epsC coding sequence hybridizes under at least low, medium, medium-high, high, or very high stringency conditions with the full-length complementary strand of SEQ ID NO: 7, 8, or 9.

In some embodiments, the endogenous epsC coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 7, 8, or 9. In some embodiments, the endogenous epsC coding sequence comprises or consists of SEQ ID NO: 7, 8, or 9.

In some aspects, the mutant produces at least 25% less (e.g., at least 50% less, at least 60% less, at least 70% less, at least 80% less, at least 90% less, or 100% less) of the polypeptide encoded by the endogenous epsC coding sequence compared to the parent Bacillus strain that lacks disruption of the endogenous epsA-O operon, when cultivated under identical conditions.

epsD

In some aspects, disruption of the endogenous epsA-O operon occurs in an endogenous epsD coding sequence. In one aspect, the endogenous epsD coding sequence is inactivated.

Examples of endogenous Bacillus epsD coding sequences include the Bacillus amyloliquefaciens epsD coding sequence of SEQ ID NO: 10 (which encodes the polypeptide of SEQ ID NO: 55), the Bacillus licheniformis epsD coding sequence of SEQ ID NO: 11 (which encodes the polypeptide of SEQ ID NO: 56), and the Bacillus subtilis epsD coding sequence of SEQ ID NO: 12 (which encodes the polypeptide of SEQ ID NO: 57).

In some embodiments, the endogenous epsD coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 55, 56, or 57; (b) hybridizes under at least low, medium, medium-high, high, or very high stringency conditions with the full-length complementary strand of SEQ ID NO: 10, 11, or 12; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 10, 11, or 12.

In some embodiments, the endogenous epsD coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 55, 56, or 57. In some embodiments, the endogenous epsD coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 55, 56, or 57. In some embodiments, the endogenous epsD coding sequence encodes for a polypeptide comprising or consisting of SEQ ID NO: 55, 56, or 57.

In some embodiments, the endogenous epsD coding sequence hybridizes under at least low, medium, medium-high, high, or very high stringency conditions with the full-length complementary strand of SEQ ID NO: 10, 11, or 12.

In some embodiments, the endogenous epsD coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 10, 11, or 12. In some embodiments, the endogenous epsD coding sequence comprises or consists of SEQ ID NO: 10, 11, or 12.

In some aspects, the mutant produces at least 25% less (e.g., at least 50% less, at least 60% less, at least 70% less, at least 80% less, at least 90% less, or 100% less) of the polypeptide encoded by the endogenous epsD coding sequence compared to the parent Bacillus strain that lacks disruption of the endogenous epsA-O operon, when cultivated under identical conditions.

epsE

In some aspects, disruption of the endogenous epsA-O operon occurs in an epsE coding sequence. In one aspect, the epsE coding sequence is inactivated.

Examples of endogenous Bacillus epsE coding sequences include the Bacillus amyloliquefaciens epsE coding sequence of SEQ ID NO: 13 (which encodes the polypeptide of SEQ ID NO: 58), the Bacillus licheniformis epsE coding sequence of SEQ ID NO: 14 (which encodes the polypeptide of SEQ ID NO: 59), and the Bacillus subtilis epsE coding sequence of SEQ ID NO: 15 (which encodes the polypeptide of SEQ ID NO: 60).

In some embodiments, the endogenous epsE coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 58, 59, or 60; (b) hybridizes under at least low, medium, medium-high, high, or very high stringency conditions with the full-length complementary strand of SEQ ID NO: 13, 14, or 15; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 13, 14, or 15.

In some embodiments, the endogenous epsE coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 58, 59, or 60. In some embodiments, the endogenous epsE coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 58, 59, or 60. In some embodiments, the endogenous epsE coding sequence encodes for a polypeptide comprising or consisting of SEQ ID NO: 58, 59, or 60.

In some embodiments, the endogenous epsE coding sequence hybridize under at least low, medium, medium-high, high, or very high stringency conditions with the full-length complementary strand of SEQ ID NO: 10, 11, or 12.

In some embodiments, the endogenous epsE coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 10, 11, or 12. In some embodiments, the endogenous epsE coding sequence comprises or consists of SEQ ID NO: 10, 11, or 12.

In some aspects, the mutant produces at least 25% less (e.g., at least 50% less, at least 60% less, at least 70% less, at least 80% less, at least 90% less, or 100% less) of the polypeptide encoded by the endogenous epsE coding sequence compared to the parent Bacillus strain that lacks disruption of the endogenous epsA-O operon, when cultivated under identical conditions.

epsF

In some aspects, disruption of the endogenous epsA-O operon occurs in an epsF coding sequence. In one aspect, the epsF coding sequence is inactivated.

Examples of Bacillus epsF coding sequences include the Bacillus amyloliquefaciens epsF coding sequence of SEQ ID NO: 16 (which encodes the polypeptide of SEQ ID NO: 61), the Bacillus licheniformis epsF coding sequence of SEQ ID NO: 17 (which encodes the polypeptide of SEQ ID NO: 62), and the Bacillus subtilis epsF coding sequence of SEQ ID NO: 18 (which encodes the polypeptide of SEQ ID NO: 63).

In some embodiments, the endogenous epsF coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 61, 62, or 63; (b) hybridizes under at least low, medium, medium-high, high, or very high stringency conditions with the full-length complementary strand of SEQ ID NO: 16, 17, or 18; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 16, 17, or 18.

In some embodiments, the endogenous epsF coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 61, 62, or 63. In some embodiments, the endogenous epsF coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 61, 62, or 63. In some embodiments, the endogenous epsF coding sequence encodes for a polypeptide comprising or consisting of SEQ ID NO: 61, 62, or 63.

In some embodiments, the endogenous epsF coding sequence hybridizes under at least low, medium, medium-high, high, or very high stringency conditions with the full-length complementary strand of SEQ ID NO: 16, 17, or 18.

In some embodiments, the endogenous epsF coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 16, 17, or 18. In some embodiments, the endogenous epsF coding sequence comprises or consists of SEQ ID NO: 16, 17, or 18.

In some aspects, the mutant produces at least 25% less (e.g., at least 50% less, at least 60% less, at least 70% less, at least 80% less, at least 90% less, or 100% less) of the polypeptide encoded by the endogenous epsF coding sequence compared to the parent Bacillus strain that lacks disruption of the endogenous epsA-O operon, when cultivated under identical conditions.

epsG

In some aspects, disruption of the endogenous epsA-O operon occurs in an epsG coding sequence. In one aspect, the epsG coding sequence is inactivated.

Examples of Bacillus epsG coding sequences include the Bacillus amyloliquefaciens epsG coding sequence of SEQ ID NO: 19 (which encodes the polypeptide of SEQ ID NO: 64), the Bacillus licheniformis epsG coding sequence of SEQ ID NO: 20 (which encodes the polypeptide of SEQ ID NO: 65), and the Bacillus subtilis epsG coding sequence of SEQ ID NO: 21 (which encodes the polypeptide of SEQ ID NO: 66).

In some embodiments, the endogenous epsG coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 64, 65, or 66; (b) hybridizes under at least low, medium, medium-high, high, or very high stringency conditions with the full-length complementary strand of SEQ ID NO: 19, 20, or 21; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 19, 20, or 21.

In some embodiments, the endogenous epsG coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 64, 65, or 66. In some embodiments, the endogenous epsG coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 64, 65, or 66. In some embodiments, the endogenous epsG coding sequence encodes for a polypeptide comprising or consisting of SEQ ID NO: 64, 65, or 66.

In some embodiments, the endogenous epsG coding sequence hybridizes under at least low, medium, medium-high, high, or very high stringency conditions with the full-length complementary strand of SEQ ID NO: 19, 20, or 21.

In some embodiments, the endogenous epsG coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 19, 20, or 21. In some embodiments, the endogenous epsG coding sequence comprises or consists of SEQ ID NO: 19, 20, or 21.

In some aspects, the mutant produces at least 25% less (e.g., at least 50% less, at least 60% less, at least 70% less, at least 80% less, at least 90% less, or 100% less) of the polypeptide encoded by the endogenous epsG coding sequence compared to the parent Bacillus strain that lacks disruption of the endogenous epsA-O operon, when cultivated under identical conditions.

epsH

In some aspects, disruption of the endogenous epsA-O operon occurs in an epsH coding sequence. In one aspect, the epsH coding sequence is inactivated.

Examples of Bacillus epsH coding sequences include the Bacillus amyloliquefaciens epsH coding sequence of SEQ ID NO: 22 (which encodes the polypeptide of SEQ ID NO: 67), the Bacillus licheniformis epsH coding sequence of SEQ ID NO: 23 (which encodes the polypeptide of SEQ ID NO: 68), and the Bacillus subtilis epsH coding sequence of SEQ ID NO: 24 (which encodes the polypeptide of SEQ ID NO: 69).

In some embodiments, the endogenous epsH coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 67, 68, or 69; (b) hybridizes under at least low, medium, medium-high, high, or very high stringency conditions with the full-length complementary strand of SEQ ID NO: 22, 23, or 24; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 22, 23, or 24.

In some embodiments, the endogenous epsH coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 67, 68, or 69. In some embodiments, the endogenous epsH coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 67, 68, or 69. In some embodiments, the endogenous epsH coding sequence encodes for a polypeptide comprising or consisting of SEQ ID NO: 67, 68, or 69.

In some embodiments, the endogenous epsH coding sequence hybridizes under at least low, medium, medium-high, high, or very high stringency conditions with the full-length complementary strand of SEQ ID NO: 22, 23, or 24.

In some embodiments, the endogenous epsH coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 22, 23, or 24. In some embodiments, the endogenous epsH coding sequence comprises or consists of SEQ ID NO: 22, 23, or 24.

In some aspects, the mutant produces at least 25% less (e.g., at least 50% less, at least 60% less, at least 70% less, at least 80% less, at least 90% less, or 100% less) of the polypeptide encoded by the endogenous epsH coding sequence compared to the parent Bacillus strain that lacks disruption of the endogenous epsA-O operon, when cultivated under identical conditions.

epsI

In some aspects, disruption of the endogenous epsA-O operon occurs in an epsI coding sequence. In one aspect, the epsI coding sequence is inactivated.

Examples of Bacillus epsI coding sequences include the Bacillus amyloliquefaciens epsI coding sequence of SEQ ID NO: 25 (which encodes the polypeptide of SEQ ID NO: 70), the Bacillus licheniformis epsI coding sequence of SEQ ID NO: 26 (which encodes the polypeptide of SEQ ID NO: 71), and the Bacillus subtilis epsI coding sequence of SEQ ID NO: 27 (which encodes the polypeptide of SEQ ID NO: 72).

In some embodiments, the endogenous epsI coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 70, 71, or 72; (b) hybridizes under at least low, medium, medium-high, high, or very high stringency conditions with the full-length complementary strand of SEQ ID NO: 25, 26, or 27; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 25, 26, or 27.

In some embodiments, the endogenous epsI coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 70, 71, or 72. In some embodiments, the endogenous epsI coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 70, 71, or 72. In some embodiments, the endogenous epsI coding sequence encodes for a polypeptide comprising or consisting of SEQ ID NO: 70, 71, or 72.

In some embodiments, the endogenous epsI coding sequence hybridize under at least low, medium, medium-high, high, or very high stringency conditions with the full-length complementary strand of SEQ ID NO: 25, 26, or 27.

In some embodiments, the endogenous epsI coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 25, 26, or 27. In some embodiments, the endogenous epsI coding sequence comprises or consists of SEQ ID NO: 25, 26, or 27.

In some aspects, the mutant produces at least 25% less (e.g., at least 50% less, at least 60% less, at least 70% less, at least 80% less, at least 90% less, or 100% less) of the polypeptide encoded by the endogenous epsI coding sequence compared to the parent Bacillus strain that lacks disruption of the endogenous epsA-O operon, when cultivated under identical conditions.

epsJ

In some aspects, disruption of the endogenous epsA-O operon occurs in an epsJ coding sequence. In one aspect, the epsJ coding sequence is inactivated.

Examples of Bacillus epsJ coding sequences include the Bacillus amyloliquefaciens epsJ coding sequence of SEQ ID NO: 28 (which encodes the polypeptide of SEQ ID NO: 73), the Bacillus licheniformis epsJ coding sequence of SEQ ID NO: 29 (which encodes the polypeptide of SEQ ID NO: 74), and the Bacillus subtilis epsJ coding sequence of SEQ ID NO: 30 (which encodes the polypeptide of SEQ ID NO: 75).

In some embodiments, the endogenous epsJ coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 73, 74, or 75; (b) hybridizes under at least low, medium, medium-high, high, or very high stringency conditions with the full-length complementary strand of SEQ ID NO: 28, 29, or 30; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 28, 29, or 30.

In some embodiments, the endogenous epsJ coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 73, 74, or 75. In some embodiments, the endogenous epsJ coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 73, 74, or 75. In some embodiments, the endogenous epsJ coding sequence encodes for a polypeptide comprising or consisting of SEQ ID NO: 73, 74, or 75.

In some embodiments, the endogenous epsJ coding sequence hybridizes under at least low, medium, medium-high, high, or very high stringency conditions with the full-length complementary strand of SEQ ID NO: 28, 29, or 30.

In some embodiments, the endogenous epsJ coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 28, 29, or 30. In some embodiments, the endogenous epsJ coding sequence comprises or consists of SEQ ID NO: 28, 29, or 30.

In some aspects, the mutant produces at least 25% less (e.g., at least 50% less, at least 60% less, at least 70% less, at least 80% less, at least 90% less, or 100% less) of the polypeptide encoded by the endogenous epsJ coding sequence compared to the parent Bacillus strain that lacks disruption of the endogenous epsA-O operon, when cultivated under identical conditions.

epsK

In some aspects, disruption of the endogenous epsA-O operon occurs in an epsK coding sequence. In one aspect, the epsK coding sequence is inactivated.

Examples of Bacillus epsK coding sequences include the Bacillus amyloliquefaciens epsK coding sequence of SEQ ID NO: 31 (which encodes the polypeptide of SEQ ID NO: 76), the Bacillus licheniformis epsK coding sequence of SEQ ID NO: 32 (which encodes the polypeptide of SEQ ID NO: 77), and the Bacillus subtilis epsK coding sequence of SEQ ID NO: 33 (which encodes the polypeptide of SEQ ID NO: 78).

In some embodiments, the endogenous epsK coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 76, 77, or 78; (b) hybridizes under at least low, medium, medium-high, high, or very high stringency conditions with the full-length complementary strand of SEQ ID NO: 31, 32, or 33; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 31, 32, or 33.

In some embodiments, the endogenous epsK coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 76, 77, or 78. In some embodiments, the endogenous epsK coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 76, 77, or 78. In some embodiments, the endogenous epsK coding sequence encodes for a polypeptide comprising or consisting of SEQ ID NO: 76, 77, or 78.

In some embodiments, the endogenous epsK coding sequence hybridizes under at least low, medium, medium-high, high, or very high stringency conditions with the full-length complementary strand of SEQ ID NO: 31, 32, or 33.

In some embodiments, the endogenous epsK coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 31, 32, or 33. In some embodiments, the endogenous epsK coding sequence comprises or consists of SEQ ID NO: 31, 32, or 33.

In some aspects, the mutant produces at least 25% less (e.g., at least 50% less, at least 60% less, at least 70% less, at least 80% less, at least 90% less, or 100% less) of the polypeptide encoded by the endogenous epsK coding sequence compared to the parent Bacillus strain that lacks disruption of the endogenous epsA-O operon, when cultivated under identical conditions.

epsL

In some aspects, disruption of the endogenous epsA-O operon occurs in an epsL coding sequence. In one aspect, the epsL coding sequence is inactivated.

Examples of Bacillus epsL coding sequences include the Bacillus amyloliquefaciens epsL coding sequence of SEQ ID NO: 34 (which encodes the polypeptide of SEQ ID NO: 79), the Bacillus licheniformis epsL coding sequence of SEQ ID NO: 35 (which encodes the polypeptide of SEQ ID NO: 80), and the Bacillus subtilis epsL coding sequence of SEQ ID NO: 36 (which encodes the polypeptide of SEQ ID NO: 81).

In some embodiments, the endogenous epsL coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 79, 80, or 81; (b) hybridizes under at least low, medium, medium-high, high, or very high stringency conditions with the full-length complementary strand of SEQ ID NO: 34, 35, or 36; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 34, 35, or 36.

In some embodiments, the endogenous epsL coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 79, 80, or 81. In some embodiments, the endogenous epsL coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 79, 80, or 81. In some embodiments, the endogenous epsL coding sequence encodes for a polypeptide comprising or consisting of SEQ ID NO: 79, 80, or 81.

In some embodiments, the endogenous epsL coding sequence hybridizes under at least low, medium, medium-high, high, or very high stringency conditions with the full-length complementary strand of SEQ ID NO: 34, 35, or 36.

In some embodiments, the endogenous epsL coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 34, 35, or 36. In some embodiments, the endogenous epsL coding sequence comprises or consists of SEQ ID NO: 34, 35, or 36.

In some aspects, the mutant produces at least 25% less (e.g., at least 50% less, at least 60% less, at least 70% less, at least 80% less, at least 90% less, or 100% less) of the polypeptide encoded by the endogenous epsL coding sequence compared to the parent Bacillus strain that lacks disruption of the endogenous epsA-O operon, when cultivated under identical conditions.

epsM

In some aspects, disruption of the endogenous epsA-O operon occurs in an epsM coding sequence. In one aspect, the epsM coding sequence is inactivated.

Examples of Bacillus epsM coding sequences include the Bacillus amyloliquefaciens epsM coding sequence of SEQ ID NO: 37 (which encodes the polypeptide of SEQ ID NO: 82), the Bacillus licheniformis epsM coding sequence of SEQ ID NO: 38 (which encodes the polypeptide of SEQ ID NO: 83), and the Bacillus subtilis epsM coding sequence of SEQ ID NO: 39 (which encodes the polypeptide of SEQ ID NO: 84).

In some embodiments, the endogenous epsM coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 82, 83, or 84; (b) hybridizes under at least low, medium, medium-high, high, or very high stringency conditions with the full-length complementary strand of SEQ ID NO: 37, 38, or 39; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 37, 38, or 39.

In some embodiments, the endogenous epsM coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 82, 83, or 84. In some embodiments, the endogenous epsM coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 82, 83, or 84. In some embodiments, the endogenous epsM coding sequence encodes for a polypeptide comprising or consisting of SEQ ID NO: 82, 83, or 84.

In some embodiments, the endogenous epsM coding sequence hybridizes under at least low, medium, medium-high, high, or very high stringency conditions with the full-length complementary strand of SEQ ID NO: 37, 38, or 39.

In some embodiments, the endogenous epsM coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 37, 38, or 39. In some embodiments, the endogenous epsM coding sequence comprises or consists of SEQ ID NO: 37, 38, or 39.

In some aspects, the mutant produces at least 25% less (e.g., at least 50% less, at least 60% less, at least 70% less, at least 80% less, at least 90% less, or 100% less) of the polypeptide encoded by the endogenous epsM coding sequence compared to the parent Bacillus strain that lacks disruption of the endogenous epsA-O operon, when cultivated under identical conditions.

epsN

In some aspects, disruption of the endogenous epsA-O operon occurs in an epsN coding sequence. In one aspect, the epsN coding sequence is inactivated.

Examples of Bacillus epsN coding sequences include the Bacillus amyloliquefaciens epsN coding sequence of SEQ ID NO: 40 (which encodes the polypeptide of SEQ ID NO: 85), the Bacillus licheniformis epsN coding sequence of SEQ ID NO: 41 (which encodes the polypeptide of SEQ ID NO: 86), and the Bacillus subtilis epsN coding sequence of SEQ ID NO: 42 (which encodes the polypeptide of SEQ ID NO: 87).

In some embodiments, the endogenous epsN coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 85, 86, or 87; (b) hybridizes under at least low, medium, medium-high, high, or very high stringency conditions with the full-length complementary strand of SEQ ID NO: 40, 41, or 42; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 40, 41, or 42.

In some embodiments, the endogenous epsN coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 85, 86, or 87. In some embodiments, the epsN coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 85, 86, or 87. In some embodiments, the endogenous epsN coding sequence encodes for a polypeptide comprising or consisting of SEQ ID NO: 85, 86, or 87.

In some embodiments, the endogenous epsN coding sequence hybridizes under at least low, medium, medium-high, high, or very high stringency conditions with the full-length complementary strand of SEQ ID NO: 40, 41, or 42.

In some embodiments, the endogenous epsN coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 40, 41, or 42. In some embodiments, the endogenous epsN coding sequence comprises or consists of SEQ ID NO: 40, 41, or 42.

In some aspects, the mutant produces at least 25% less (e.g., at least 50% less, at least 60% less, at least 70% less, at least 80% less, at least 90% less, or 100% less) of the polypeptide encoded by the endogenous epsN coding sequence compared to the parent Bacillus strain that lacks disruption of the endogenous epsA-O operon, when cultivated under identical conditions.

epsO

In some aspects, disruption of the endogenous epsA-O operon occurs in an epsO coding sequence. In one aspect, the epsO coding sequence is inactivated.

Examples of Bacillus epsO coding sequences include the Bacillus amyloliquefaciens epsO coding sequence of SEQ ID NO: 43 (which encodes the polypeptide of SEQ ID NO: 88), the Bacillus licheniformis epsO coding sequence of SEQ ID NO: 44 (which encodes the polypeptide of SEQ ID NO: 89), and the Bacillus subtilis epsO coding sequence of SEQ ID NO: 45 (which encodes the polypeptide of SEQ ID NO: 90).

In some embodiments, the endogenous epsO coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 88, 89, or 90; (b) hybridizes under at least low, medium, medium-high, high, or very high stringency conditions with the full-length complementary strand of SEQ ID NO: 43, 44, or 45; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 43, 44, or 45.

In some embodiments, the endogenous epsO coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 88, 89, or 90. In some embodiments, the endogenous epsO coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 88, 89, or 90. In some embodiments, the endogenous epsO coding sequence encodes for a polypeptide comprising or consisting of SEQ ID NO: 88, 89, or 90.

In some embodiments, the endogenous epsO coding sequence hybridizes under at least low, medium, medium-high, high, or very high stringency conditions with the full-length complementary strand of SEQ ID NO: 43, 44, or 45.

In some embodiments, the endogenous epsO coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 43, 44, or 45. In some embodiments, the endogenous epsO coding sequence comprises or consists of SEQ ID NO: 43, 44, or 45.

In some aspects, the mutant produces at least 25% less (e.g., at least 50% less, at least 60% less, at least 70% less, at least 80% less, at least 90% less, or 100% less) of the polypeptide encoded by the endogenous epsO coding sequence compared to the parent Bacillus strain that lacks disruption of the endogenous epsA-O operon, when cultivated under identical conditions.

mecA

Disruption of the mecA gene has been shown to contribute to increased transformation efficiency in Bacillus hosts (see WO 2014/052630, the content of which is hereby incorporated by reference; and Hahn et al., 1995, Mol. Microbiol. 18: 755-767). Accordingly, in some aspects, the Bacillus mutants further comprise a disruption of an endogenous mecA gene. Disruption of the endogenous mecA gene may occur in the coding sequence and/or promoter sequence. In one aspect, the mecA gene is inactivated.

Examples of endogenous mecA genes include a Bacillus amyloliquefaciens mecA gene comprising the coding sequence of SEQ ID NO: 91 (encoding the polypeptide of SEQ ID NO: 92), a Bacillus licheniformis mecA gene comprising the coding sequence of SEQ ID NO: 93 (encoding the polypeptide of SEQ ID NO: 94), and a Bacillus subtilis mecA gene comprising the coding sequence of SEQ ID NO: 95 (encoding the polypeptide of SEQ ID NO: 96).

In some embodiments, the endogenous mecA coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 92, 94, or 96; (b) hybridizes under at least low, medium, medium-high, high, or very high stringency conditions with the full-length complementary strand of SEQ ID NO: 91, 93, or 95; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 91, 93, or 95.

In some embodiments, the endogenous mecA coding sequence encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 92, 94, or 96. In some embodiments, the endogenous epsA coding sequence encodes for a polypeptide having a sequence that differs by no more than ten amino acids, e.g., by no more than five amino acids, by no more than four amino acids, by no more than three amino acids, by no more than two amino acids, or by one amino acid from any of SEQ ID NOs: 92, 94, or 96. In some embodiments, the endogenous mecA coding sequence encodes for a polypeptide comprising or consisting of SEQ ID NO: 92, 94, or 96.

In some embodiments, the endogenous mecA coding sequence hybridizes under at least low, medium, medium-high, high, or very high stringency conditions with the full-length complementary strand of SEQ ID NO: 91, 93, or 95.

In some embodiments, the endogenous mecA coding sequence has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 91, 93, or 95. In some embodiments, the endogenous epsA coding sequence comprises or consists of SEQ ID NO: 91, 93, or 95.

In some aspects, the mutant produces at least 25% less (e.g., at least 50% less, at least 60% less, at least 70% less, at least 80% less, at least 90% less, or 100% less) of the polypeptide encoded by the endogenous mecA coding sequence compared to the parent Bacillus strain that lacks disruption of the endogenous mecA gene, when cultivated under identical conditions.

In some aspects, the mutant has improved transformation efficiency compared to the parent Bacillus strain that lacks disruption of the endogenous mecA gene, when cultivated under identical conditions. In some embodiments, the mutant is capable of producing at least 10-fold (e.g., at least 100-fold, at least 1000-fold, at least 10000-fold, or at least 100000-fold) more transformants compared to the parent Bacillus strain that lacks disruption of the endogenous mecA gene, when cultivated under identical conditions.

The polynucleotide sequences disclosed herein, or subsequences thereof, as well as the amino acid sequences described herein, or fragments thereof, may be used to design nucleic acid probes to identify and clone homologous coding sequences in epsA-O operons from strains of different genera or species according to methods well known in the art. In particular, such probes can be used for hybridization with the DNA from a Bacillus species of interest, following standard Southern blotting procedures, in order to identify and isolate the corresponding gene therein. Such probes can be considerably shorter than the entire sequence, e.g., at least 14 nucleotides, at least 25 nucleotides, at least 35 nucleotides, at least 70 nucleotides in lengths. The probes may be longer, e.g., at least 100 nucleotides, at least 200 nucleotides, at least 300 nucleotides, at least 400 nucleotides, at least 500 nucleotides in lengths. Even longer probes may be used, e.g., at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin).

A DNA library prepared from such other strains may be screened for DNA that hybridizes with the probes described above. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with the polynucleotide sequences described herein, or a subsequence thereof, the carrier material may be used in a Southern blot. For purposes of the probes described above, hybridization indicates that the polynucleotide hybridizes to a labeled nucleic acid probe corresponding to the polynucleotide sequences, the full-length complementary strand thereof, or a subsequence of the foregoing; under very low to very high stringency conditions. Molecules to which the nucleic acid probe hybridizes under these conditions can be detected using, for example, X-ray film.

For long probes of at least 100 nucleotides in length, very low to very high stringency conditions are defined as prehybridization and hybridization at 42° C. in 5×SSPE, 0.3% SDS, 200 micrograms/mL sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally. The carrier material is finally washed three times each for 15 minutes using 0.2×SSC, 0.2% SDS at 45° C. (very low stringency), at 50° C. (low stringency), at 55° C. (medium stringency), at 60° C. (medium-high stringency), at 65° C. (high stringency), and at 70° C. (very high stringency).

For short probes of about 15 nucleotides to about 70 nucleotides in length, stringency conditions are defined as prehybridization and hybridization at about 5° C. to about 10° C. below the calculated Tₘ using the calculation according to Bolton and McCarthy (Proc. Natl. Acad. Sci. USA 48: 1390 (1962)) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per mL following standard Southern blotting procedures for 12 to 24 hours optimally. The carrier material is finally washed once in 6×SCC plus 0.1% SDS for 15 minutes and twice each for 15 minutes using 6×SSC at 5° C. to 10° C. below the calculated Tₘ.

Homologs of the polypeptides encoded by the endogenous coding sequences of the epsA-O operons described herein from strains of different genera or species generally have amino acid changes that are of a minor nature, that is conservative amino acid substitutions or insertions that do not significantly affect the folding and/or activity of the protein; small deletions, typically of one to about 30 amino acids; small amino-terminal or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with other related enzymes.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnol. 17: 893-896). Mutagenized DNA molecules that encode active enzymes can be recovered from the host cells and rapidly sequenced using standard methods in the art. These methods allow the rapid determination of the importance of individual amino acid residues in a polypeptide.

Disruption of the epsA-O Operon and Methods of Producing Bacillus Mutants

The Bacillus mutant strains described herein may be constructed by disrupting the referenced endogenous epsA-O operon using methods well known in the art, including those methods described herein. A portion of the operon can be disrupted such as one or more (e.g., two, several) coding regions or a control sequence required for expression of the coding regions. Such a control sequence of the operon may be a promoter sequence or a functional part thereof, i.e., a part that is sufficient for affecting expression of the operon. For example, a promoter sequence may be inactivated resulting in no expression or a weaker promoter may be substituted for the native promoter sequence to reduce expression of the coding sequences. Other control sequences for possible modification include, but are not limited to, a leader, propeptide sequence, signal sequence, transcription terminator, and transcriptional activator.

The Bacillus mutant strains may be constructed by gene deletion techniques to eliminate or reduce expression of the epsA-O operon coding sequences. Gene deletion techniques enable the partial or complete removal of the operon thereby eliminating expression. In such methods, deletion of the operon is accomplished by homologous recombination using one or more plasmids that have been constructed to contiguously contain the 5′ and 3′ regions flanking the genes.

The Bacillus mutant strains may also be constructed by introducing, substituting, and/or removing one or more (e.g., two, several) nucleotides in the epsA-O operon or a control sequence thereof required for the transcription or translation thereof. For example, nucleotides may be inserted or removed for the introduction of a stop codon, the removal of the start codon, or a frame-shift of the open reading frame. Such a modification may be accomplished by site-directed mutagenesis or PCR generated mutagenesis in accordance with methods known in the art. See, for example, Botstein and Shortie, 1985, Science 229: 4719; Lo et al., 1985, Proc. Natl. Acad. Sci. USA 81: 2285; Higuchi et al., 1988, Nucleic Acids Res 16: 7351; Shimada, 1996, Meth. Mol. Biol. 57: 157; Ho et al., 1989, Gene 77: 51; Horton et al., 1989, Gene 77: 61; and Sarkar and Sommer, 1990, Bio Techniques 8: 404.

The Bacillus mutant strains may also be constructed by gene disruption techniques by inserting into the epsA-O operon a disruptive nucleic acid construct comprising a nucleic acid fragment homologous to the coding sequence that will create a duplication of the region of homology and incorporate construct DNA between the duplicated regions. Such a gene disruption can eliminate expression if the inserted construct separates the promoter of the operon from the coding regions or interrupts the coding sequences such that a non-functional or functionally reduced coding sequence results. A disrupting construct may be simply a selectable marker gene accompanied by 5′ and 3′ regions homologous to the gene. The selectable marker enables identification of transformants containing the disrupted operon.

The Bacillus mutant strains may also be constructed by the process of gene conversion (see, for example, Iglesias and Trautner, 1985, Molecular General Genetics 189: 73-76). For example, in the gene conversion method, a nucleotide sequence corresponding to a sequence in the epsA-O operon is mutagenized in vitro to produce a defective nucleotide sequence, which is then transformed into the parent Bacillus strain to produce a defective operon. By homologous recombination, the defective nucleotide sequence replaces the endogenous sequence. It may be desirable that the defective nucleotide sequence also comprises a marker for selection of transformants containing the defective sequence.

The Bacillus mutant strains may be further constructed by random or specific mutagenesis using methods well known in the art, including, but not limited to, chemical mutagenesis (see, for example, Hopwood, The Isolation of Mutants in Methods in Microbiology (J. R. Norris and D. W. Ribbons, eds.) pp. 363-433, Academic Press, New York, 1970). Modification of the epsA-O operon may be performed by subjecting the parent strain to mutagenesis and screening for mutant strains in which expression of the gene has been reduced or inactivated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosogaunidine (NTG) O-methyl hydroxylamine, nitrous acid, ethyl methane sulphonate (EMS), sodium bisulphite, formic acid, and nucleotide analogues. When such agents are used, the mutagenesis is typically performed by incubating the parent strain to be mutagenized in the presence of the mutagenizing agent of choice under suitable conditions, and selecting for mutants exhibiting reduced or no expression of the coding sequences in the epsA-O operon.

A nucleotide sequence homologous or complementary to a sequence described herein may be used from other microbial sources to disrupt the corresponding sequence in a Bacillus strain of choice.

In one aspect, disruption of the epsA-O operon in the Bacillus mutant is unmarked with a selectable marker. Removal of the selectable marker gene may be accomplished by culturing the mutants on a counter-selection medium. Where the selectable marker gene contains repeats flanking its 5′ and 3′ ends, the repeats will facilitate the looping out of the selectable marker gene by homologous recombination when the mutant strain is submitted to counter-selection. The selectable marker gene may also be removed by homologous recombination by introducing into the mutant strain a nucleic acid fragment comprising 5′ and 3′ regions of the defective gene, but lacking the selectable marker gene, followed by selecting on the counter-selection medium. By homologous recombination, the defective gene containing the selectable marker gene is replaced with the nucleic acid fragment lacking the selectable marker gene. Other methods known in the art may also be used.

Also described are methods of producing the Bacillus mutant described herein. In one aspect is a method for obtaining a Bacillus mutant described herein, comprising disrupting an endogenous epsA-O operon in a parent Bacillus strain. In another aspect is a method for obtaining a Bacillus mutant described herein, comprising: (a) cultivating a parent Bacillus strain; (b) disrupting an endogenous epsA-O operon in a parent Bacillus strain of (a); and (c) isolating the mutant strain resulting from (b).

### Methods of Using the Bacillus Mutants

The Bacillus mutants described herein are useful for producing Bacillus transformants. One aspect is a method of obtaining a Bacillus transformant, comprising transforming a heterologous polynucleotide into a Bacillus mutant described herein. Another aspect is a method of obtaining a Bacillus transformant, comprising: (a) cultivating a Bacillus mutant described herein (e.g., a Bacillus mutant comprising a disruption of an endogenous epsA-O operon); (b) transforming a heterologous polynucleotide into the Bacillus mutant of (a); and (c) isolating the transformant strain resulting from (b).

The transformed DNA described herein can be any DNA of interest. The DNA may be of genomic, cDNA, semisynthetic, synthetic origin, or any combinations thereof. The DNA may be a heterologous polynucleotide that encodes any polypeptide having biological activity of interest or may be a DNA involved in the expression of the polypeptide having biological activity, e.g., a promoter.

The polypeptide having a biological activity may be any polypeptide of interest. The polypeptide may be native or foreign to the Bacillus host cell of interest. The polypeptide may be naturally occurring allelic and engineered variations of the below-mentioned polypeptides and hybrid polypeptides.

The term “polypeptide” is not meant herein to refer to a specific length of the encoded product and, therefore, encompasses peptides, oligopeptides, and proteins. The term “polypeptide” also encompasses hybrid polypeptides, which comprise a combination of partial or complete polypeptide sequences obtained from at least two different polypeptides wherein one or more may be foreign to the Bacillus cell. Polypeptides further include naturally occurring allelic and engineered variations of a polypeptide.

In one aspect, the polypeptide is an antibody, antigen, antimicrobial peptide, enzyme, growth factor, hormone, immunodilator, neurotransmitter, receptor, reporter protein, structural protein, and transcription factor.

In another aspect, the polypeptide is a hydrolase, isomerase, ligase, lyase, oxidoreductase, or transferase. In a most preferred aspect, the polypeptide is an alpha-glucosidase, aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, glucocerebrosidase, alpha-glucosidase, beta-glucosidase, invertase, laccase, lipase, mannosidase, mutanase, oxidase, pectinolytic enzyme, peroxidase, phospholipase, phytase, polyphenoloxidase, proteolytic enzyme, ribonuclease, transglutaminase, urokinase, or xylanase.

In another aspect, the polypeptide is an albumin, collagen, tropoelastin, elastin, or gelatin.

In another aspect, the polypeptide is a hybrid polypeptide, which comprises a combination of partial or complete polypeptide sequences obtained from at least two different polypeptides wherein one or more may be foreign to the Bacillus host cell.

In another aspect, the polypeptide is a fused polypeptide in which another polypeptide is fused at the N-terminus or the C-terminus of the polypeptide or fragment thereof. A fused polypeptide is produced by fusing a nucleotide sequence (or a portion thereof) encoding one polypeptide to a nucleotide sequence (or a portion thereof) encoding another polypeptide. Techniques for producing fusion polypeptides are known in the art, and include, ligating the coding sequences encoding the polypeptides so that they are in frame and expression of the fused polypeptide is under control of the same promoter(s) and terminator.

The heterologous polynucleotide encoding a polypeptide of interest may be obtained from any prokaryotic, eukaryotic, or other source. For purposes of the present invention, the term “obtained from” as used herein in connection with a given source shall mean that the polypeptide is produced by the source or by a cell in which a gene from the source has been inserted.

Techniques used to isolate or clone a heterologous polynucleotide encoding a polypeptide of interest are known in the art and include isolation from genomic DNA, preparation from cDNA, or a combination thereof. The cloning of the DNA of interest from such genomic DNA can be effected, e.g., by using the well known polymerase chain reaction (PCR). See, for example, Innis et al., PCR Protocols: A Guide to Methods and Application, Academic Press, New York, 1990. The cloning procedures may involve excision and isolation of a desired nucleic acid fragment comprising the nucleic acid sequence encoding the polypeptide, insertion of the fragment into a vector molecule, and incorporation of the recombinant vector into the Bacillus mutant where multiple copies or clones of the nucleic acid sequence will be replicated. The DNA may be of genomic, cDNA, RNA, semisynthetic, synthetic origin, or any combinations thereof.

A heterologous polynucleotide encoding a polypeptide of interest may be manipulated in a variety of ways to provide for expression of the polypeptide in a mutant Bacillus strain. Manipulation of the polynucleotide's sequence prior to its insertion into a vector may be desirable or necessary depending on the expression vector. The techniques for modifying polynucleotide sequences utilizing recombinant DNA methods are well known in the art.

A nucleic acid construct comprising a polynucleotide encoding a polypeptide may be operably linked to one or more (e.g., two, several) control sequences capable of directing expression of the coding sequence in a mutant Bacillus strain of the present invention under conditions compatible with the control sequences.

The control sequence may be an appropriate promoter sequence, a nucleotide sequence that is recognized by a mutant Bacillus strain of the present invention for expression of the polynucleotide encoding the polypeptide. The promoter sequence contains transcriptional control sequences that mediate expression of the polypeptide. The promoter may be any nucleotide sequence that shows transcriptional activity in the mutant Bacillus strain, including mutant, truncated, and hybrid promoters, and may be obtained from genes encoding extracellular or intracellular polypeptides either native or foreign to the mutant Bacillus strain.

Examples of suitable promoters for directing the transcription of the nucleic acid constructs in a mutant Bacillus strain are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, Molecular Cloning, A Laboratory Manual, 2d edition, Cold Spring Harbor, N.Y.

The control sequence may also be a suitable transcription terminator sequence, a sequence recognized by a mutant Bacillus strain to terminate transcription. The terminator sequence is operably linked to the 3′ terminus of the nucleotide sequence encoding the heterologous polypeptide. Any terminator that is functional in a Bacillus strain may be used.

The control sequence may also be a suitable leader sequence, a nontranslated region of mRNA that is important for translation by a mutant Bacillus strain. The leader sequence is operably linked to the 5′ terminus of the nucleotide sequence encoding the heterologous polypeptide. Any leader sequence that is functional in the mutant Bacillus strain may be used.

The control sequence may also be a signal peptide coding sequence that codes for an amino acid sequence linked to the amino terminus of a polypeptide and directs the encoded polypeptide into the cell's secretory pathway. The 5′ end of the coding sequence of the nucleotide sequence may inherently contain a signal peptide coding sequence naturally linked in translation reading frame with the segment of the coding sequence that encodes the secreted polypeptide. Alternatively, the 5′ end of the coding sequence may contain a signal peptide coding sequence that is foreign to the coding sequence. The foreign signal peptide coding sequence may be required where the coding sequence does not naturally contain a signal peptide coding sequence. Alternatively, the foreign signal peptide coding sequence may simply replace the natural signal peptide coding sequence in order to enhance secretion of the polypeptide. However, any signal peptide coding sequence that directs the expressed polypeptide into the secretory pathway of the mutant Bacillus strain, i.e., secreted into a culture medium, may be used in the present invention.

A recombinant expression vector comprising a nucleotide sequence, a promoter, and transcriptional and translational stop signals may be used for the recombinant production of a polypeptide of interest. The various nucleic acids and control sequences described herein may be joined together to produce a recombinant expression vector that may include one or more (e.g., two, several) convenient restriction sites to allow for insertion or substitution of the nucleotide sequence encoding the polypeptide at such sites. Alternatively, the nucleotide sequence may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the nucleotide sequence. The choice of the vector will typically depend on its compatibility with the mutant Bacillus strain into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the mutant Bacillus strain, is integrated into the genome and replicated together with the chromosome(s) into which it has been integrated. Furthermore, a single vector or plasmid or two or more vectors or plasmids that together contain the total DNA to be introduced into the genome of the mutant Bacillus strain, or a transposon, may be used.

The vector may contain one or more (e.g., two, several) selectable markers that permit easy selection of transformed mutant Bacillus strains. A selectable marker is a gene the product of which provides for biocide or viral resistance, resistance to heavy metals, prototrophy to auxotrophs, and the like.

Examples of selectable markers for use in the mutant Bacillus strain include, but are not limited to, the dal genes from Bacillus subtilis or Bacillus licheniformis, or markers that confer antibiotic resistance such as ampicillin, chloramphenicol, kanamycin, or tetracycline resistance.

The vectors may contain one or more (e.g., two, several) elements that permit integration of the vector into the Bacillus genome or autonomous replication of the vector in the cell independent of the genome.

For integration into the genome of the mutant Bacillus strain, the vector may rely on the polynucleotide's sequence encoding the polypeptide of interest or any other element of the vector for integration into the genome by homologous or nonhomologous recombination. Alternatively, the vector may contain additional nucleotide sequences for directing integration by homologous recombination into the genome of the mutant Bacillus strain at a precise location(s) in the chromosome. To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which have a high degree of identity to the corresponding target sequence to enhance the probability of homologous recombination. The integrational elements may be any sequence that is homologous with the target sequence in the genome of the mutant Bacillus strain. Furthermore, the integrational elements may be non-encoding or encoding nucleotide sequences. On the other hand, the vector may be integrated into the genome of the mutant Bacillus strain by non-homologous recombination.

For autonomous replication, the vector may further comprise an origin of replication enabling the vector to replicate autonomously in the mutant Bacillus strain. The origin of replication may be any plasmid replicator mediating autonomous replication that functions in a cell. The term “origin of replication” or “plasmid replicator” is defined herein as a nucleotide sequence that enables a plasmid or vector to replicate in vivo. Examples of bacterial origins of replication useful in the mutant Bacillus strain are the origins of replication of plasmids pBR322, pUC19, pACYC177, and pACYC184 permitting replication in E. coli, and pUB110, pE194, pTA1060, and pAMβ1 permitting replication in Bacillus.

The procedures used to ligate the elements described herein to construct the recombinant expression vectors are well known to one skilled in the art (see, e.g., J. Sambrook et al., 1989, supra).

The DNA can also be a control sequence, e.g., promoter, for manipulating the expression of a gene of interest. Non-limiting examples of control sequences are described above.

The DNA can further be a nucleic acid construct for inactivating a gene of interest in a Bacillus cell.

The DNA is not to be limited in scope by the specific examples disclosed above, since these examples are intended as illustrations of several aspects of the invention.

Transformation of the DNA into the mutant Bacillus strains can be conducted using techniques known in the art, such as electroporation as described in the Examples section below.

The transformants described herein can be isolated using standard techniques well-known in the art, including, but not limited to, streak plate isolation, growth in enrichment or selective media, temperature growth selection, filtration, or single cell isolation techniques, such as flow cytometry and microfluidics.

### Methods of Producing Polypeptides and Fermentation Products

### Polypeptides

As mentioned above, the Bacillus mutants described herein can increase the efficiency in producing Bacillus transformants which are useful, e.g., in producing a polypeptide having biological activity. Accordingly, in one aspect is a method of producing a polypeptide having biological activity, comprising: (a) cultivating a Bacillus host cell transformed with a heterologous polynucleotide encoding the polypeptide under conditions conducive for production of the polypeptide, wherein the Bacillus host cell is a Bacillus mutant described herein; and (b) recovering the polypeptide.

Another aspect is a method of producing a polypeptide, comprising: (a) cultivating a Bacillus transformant described herein (e.g., a Bacillus mutant comprising a disruption of an endogenous epsA-O operon); and (b) recovering the polypeptide.

The competent Bacillus host cells are cultivated in a nutrient medium suitable for production of a polypeptide of interest using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the polypeptide of interest to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). The secreted substance of interest, e.g., polypeptide or fermentation product, can be recovered directly from the medium or the whole broth is recovered.

The polypeptide having biological activity may be detected using methods known in the art that are specific for the substance. These detection methods may include use of specific antibodies, high performance liquid chromatography, capillary chromatography, formation of an enzyme product, disappearance of an enzyme substrate, or SDS-PAGE. For example, an enzyme assay may be used to determine the activity of a polypeptide having enzyme activity. Procedures for determining enzyme activity are known in the art for many enzymes (see, for example, D. Schomburg and M. Salzmann (eds.), Enzyme Handbook, Springer-Verlag, New York, 1990).

The resulting polypeptide having biological activity may be isolated by methods known in the art. For example, a polypeptide of interest may be isolated from the cultivation medium by conventional procedures including, but not limited to, centrifugation, filtration, extraction, spray-drying, evaporation, or precipitation. The isolated polypeptide may then be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989).

### Fermentation Products

The Bacillus mutants described herein can be used in metabolic engineering, e.g., in the production of a fermentation product. The increased transformation efficiency for the mutants may provide the tools to use Bacillus over an existing host, and may permit rapid screening of overexpressed heterologous genes for existing and new metabolic pathways.

“Fermentation” or “fermentation process” refers to any fermentation process or any process comprising a fermentation step. Fermentation processes include fermentation processes used in the consumable alcohol industry (e.g., beer and wine), dairy industry (e.g., fermented dairy products), leather industry, tobacco industry, and specialty or bulk chemical industry.

In one aspect is a method of producing a fermentation product, comprising: (a) cultivating a Bacillus transformant described herein (e.g., a Bacillus mutant described herein transformed with one or more heterologous polynucleotides that encode one or more polypeptides of a fermentation pathway) under conditions conducive for production of the fermentation product; and (b) recovering the fermentation product.

The Bacillus transformant can be any Bacillus mutant described herein that is transformed with one or more heterologous fermentation pathway genes, resulting in increased production of a desired fermentation product. Metabolic pathway genes and corresponding engineered transformants for fermentation of a variety of desired fermentation products are known in the art, e.g., the production of isopropanol and n-propanol (WO 2012/058603), 3-hydroxypropionic acid (WO 2005/118719), malic acid (WO 2011/028643), 1,4-butanediol (WO 2008/115840), 1,3-butanediol (WO 2010/127319), 2-butanol (WO 2010/144746), THF (WO 2010/141920), caprolactam (WO 2010/129936), hexamethylenediamine (WO 2010/129936), levulinic acid (WO 2010/129936), 2/3-hydroxyisobutyric acid (WO 2009/135074), methacrylic acid (WO 2009/135074), adipic acid (WO 2009/151728), butadiene (WO 2011/140171), muconate (WO 2011/017560) and 4-hydroxybutanal (WO 2011/047101) (the contents of these applications are hereby incorporated by reference). The Bacillus mutants described herein may provide tools to further improve on producing the fermented products in the references above.

Methods for producing a fermentation product may be performed in a fermentable medium comprising one or more (e.g., two, several) sugars, such as glucose, fructose, sucrose, cellobiose, xylose, xylulose, arabinose, mannose, galactose, and/or soluble oligosaccharides. In some instances, the fermentation medium is derived from a natural source, such as sugar cane, starch, or cellulose, and may be the result of pretreating the source by enzymatic hydrolysis (saccharification).

In addition to the appropriate carbon sources from one or more (e.g., two, several) sugar(s), the fermentable medium may contain other nutrients or stimulators known to those skilled in the art, such as macronutrients (e.g., nitrogen sources) and micronutrients (e.g., vitamins, mineral salts, and metallic cofactors). In some aspects, the carbon source can be preferentially supplied with at least one nitrogen source, such as yeast extract, N₂, peptone (e.g., Bacto™ Peptone), or soytone (e.g., Bacto™ Soytone). Nonlimiting examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. Examples of mineral salts and metallic cofactors include, but are not limited to Na, P, K, Mg, S, Ca, Fe, Zn, Mn, and Cu.

The fermenting microorganism is typically added to the fermentation medium and the fermentation is performed for about 8 to about 96 hours, e.g., about 24 to about 60 hours. The temperature is typically between about 26° C. to about 60° C., e.g., about 32° C. or 50°, and about pH 3 to about pH 8, e.g., pH 4-5, 6, or 7.

Cultivation may be performed under anaerobic, substantially anaerobic (microaerobic), or aerobic conditions, as appropriate. Briefly, anaerobic refers to an environment devoid of oxygen, substantially anaerobic (microaerobic) refers to an environment in which the concentration of oxygen is less than air, and aerobic refers to an environment wherein the oxygen concentration is approximately equal to or greater than that of the air. Substantially anaerobic conditions include, for example, a culture, batch fermentation or continuous fermentation such that the dissolved oxygen concentration in the medium remains less than 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases. In some embodiments, the cultivation is performed under anaerobic conditions or substantially anaerobic conditions.

The methods for producing a fermentation product can employ any suitable fermentation operation mode. For example, batch mode fermentation may be used with a close system where culture media and host microorganism, set at the beginning of fermentation, have no additional input except for the reagents certain reagents, e.g., for pH control, foam control or others required for process sustenance. The process described herein can also be employed in Fed-batch or continuous mode.

The methods for producing a fermentation product may be practiced in several bioreactor configurations, such as stirred tank, bubble column, airlift reactor and others known to those skilled in the art. The methods may be performed in free cell culture or in immobilized cell culture as appropriate. Any material support for immobilized cell culture may be used, such as alginates, fibrous bed, or argyle materials such as chrysotile, montmorillonite KSF and montmorillonite K-10.

A fermentation product can be any substance derived from the fermentation. The fermentation product can be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol); an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g., pentene, hexene, heptene, and octene); an amino acid (e.g., aspartic acid, glutamic acid, glycine, lysine, serine, and threonine); a gas (e.g., methane, hydrogen (H₂), carbon dioxide (CO₂), and carbon monoxide (CO)); isoprene; a ketone (e.g., acetone); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); and polyketide. The fermentation product can also be protein as a high value product.

In one aspect, the fermentation product is an alcohol. It will be understood that the term “alcohol” encompasses a substance that contains one or more hydroxyl moieties. The alcohol can be any alcohol, including, but not limited to propanol, n-butanol, iso-butanol, isobutanol, ethanol, methanol, arabinitol, butanediol, ethylene glycol, glycerin, glycerol, 1,3-propanediol, sorbitol, or xylitol. See, for example, Gong, et al., 1999, Ethanol production from renewable resources, in Advances in Biochemical Engineering/Biotechnology, Scheper, T., ed., Springer-Verlag Berlin Heidelberg, Germany, 65: 207-241; Silveira and Jonas, 2002, Appl. Microbiol. Biotechnol. 59: 400-408; Nigam and Singh, 1995, Process Biochemistry 30: 117-124; Ezeji et al., 2003, World Journal of Microbiology and Biotechnology 19: 595-603.

In another aspect, the fermentation product is propanol, such as isopropanol and/or n-propanol (see WO 2012/058603, the content of which is hereby incorporated by reference).

In another aspect, the fermentation product is an alkane. The alkane can be any unbranched or a branched alkane, including, but not limited to pentane, hexane, heptanes, octane, nonane, decane, undecane, or dodecane.

In another aspect, the fermentation product is a cycloalkane, e.g., cyclopentane, cyclohexane, cycloheptane, or cyclooctane.

In another aspect, the fermentation product is an alkene. The alkene can be any unbranched or a branched alkene, including, but not limited to pentene, hexane, heptene, or octene.

In another aspect, the fermentation product is an amino acid. The amino acid can be any amino acid, including, but not limited to aspartic acid, glutamic acid, glycine, lysine, serine, or threonine. See, for example, Richard and Margaritis, 2004, Biotechnol. Bioeng. 87: 501-515.

In another aspect, the fermentation product is a gas. The gas can be any gas, including, but not limited to methane, H₂, CO₂, or CO. See, for example, Kataoka et al., 1997, Water Science and Technology 36: 41-47; and Gunaseelan, 1997, Biomass and Bioenergy 13: 83-114.

In another aspect, the fermentation product is isoprene.

In another aspect, the fermentation product is a ketone. The term “ketone” encompasses a substance that contains one or more ketone moieties. In one aspect, the ketone is acetone.

In another aspect, the fermentation product is an organic acid. The organic acid can be any organic acid, including, but not limited to acetic acid, acetonic acid, adipic acid, ascorbic acid, citric acid, 2,5-diketo-D-gluconic acid, formic acid, fumaric acid. glucaric acid, gluconic acid, glucuronic acid, glutaric acid, 3-hydroxypropionic acid, itaconic acid, lactic acid, malic acid, malonic acid, oxalic acid, propionic acid, succinic acid, or xylonic acid. See, for example, Chen and Lee, 1997, Appl. Biochem. Biotechnol. 63-65: 435-448. In some aspects, the fermentation product is an amino acid. The amino acid can be any amino acid, including, but not limited to aspartic acid, glutamic acid, glycine, lysine, serine, or threonine. See, for example, Richard and Margaritis, 2004, Biotechnol. Bioeng. 87: 501-515.

In another aspect, the fermentation product is polyketide.

Suitable assays to test for the production of the fermentation product can be performed using methods known in the art, as described above for polypeptides. For example, the fermentation product (and other organic compounds, such as side products) can be analyzed by methods such as HPLC (High Performance Liquid Chromatography), GC-MS (Gas Chromatography Mass Spectroscopy) and LC-MS (Liquid Chromatography-Mass Spectroscopy) or other suitable analytical methods using routine procedures well known in the art. The release of the fermentation product in the fermentation broth can also be tested with the culture supernatant. Byproducts and residual sugar in the fermentation medium (e.g., glucose) can be quantified by HPLC using, for example, a refractive index detector for glucose and alcohols, and a UV detector for organic acids (Lin et al., 2005, Biotechnol Bioeng 90: 775-779), or using other suitable assay and detection methods well known in the art.

Recovery of the fermentation product from the fermentation medium can be conducted using any procedure known in the art including, but not limited to, chromatography (e.g., size exclusion chromatography, adsorption chromatography, ion exchange chromatography), electrophoretic procedures, differential solubility, distillation, extraction (e.g., liquid-liquid extraction), pervaporation, extractive filtration, membrane filtration, membrane separation, reverse osmosis, ultrafiltration, or crystallization.

The following examples are provided by way of illustration and are not intended to be limiting of the invention.

### EXAMPLES

Chemicals used as buffers and substrates were commercial products of at least reagent grade.

### Strains

Escherichia coli

One Shot™ TOP10 chemically competent E. coli cells (Invitrogen, Carlsbad, Calif., USA) and Sure™ Competent cells (Stratagene, La Jolla, Calif., USA) were used for routine plasmid constructions and propagation.

Bacillus licheniformis

B. licheniformis SJ1904 (U.S. Pat. No. 5,733,753) was used as a host for the disruptions described below.

Bacillus amyloliquefaciens

B. amyloliquefaciens FZB24 (Taegro®, EPA registration number: 70127-5, EPA establishment number: 33967-NJ-1) was used as a host for the disruptions described below.

### Media

Bacillus strains were grown on TBAB (Tryptose Blood Agar Base, Difco Laboratories, Sparks, Md., USA) or LB agar plates (10 g/l Tryptone, 5 g/l yeast extract, 5 g/l NaCl, 15 g/l agar) plates or in LB liquid medium (10 g/l Tryptone, 5 g/l yeast extract, 5 g/l NaCl).

To select for erythromycin resistance, agar media were supplemented with 1 μg/ml erythromycin+25 μg/ml lincomycin and liquid media were supplemented with 5 μg/ml erythromycin. To select for spectinomycin resistance, agar media were supplemented with 120 μg/ml spectinomycin. To select for chloramphenicol resistance, agar media were supplemented with 5 μg/ml chloramphenicol.

Spizizen I medium consists of 1× Spizizen salts (6 g/l KH₂PO₄, 14 g/l K₂HPO₄, 2 g/l (NH₄)₂SO₄, 1 g/l sodium citrate, 0.2 g/l MgSO₄, pH 7.0), 0.5% glucose, 0.1% yeast extract, and 0.02% casein hydrolysate.

Spizizen II medium consists of Spizizen I medium supplemented with 0.5 mM CaCl₂, and 2.5 mM MgCl₂.

### Example 1

### Construction of a B. amyloliquefaciens Strain Comprising a Disrupted epsA-O Operon (BaC0171)

This example describes the disruption of the epsA-O operon by deletion of nucleotides within the epsH and epsG coding sequences.

Plasmid pBM340 was designed to delete 421 bp within the B. amyloliquefaciens epsH gene and 217 bp within the epsG gene. Genomic DNA was isolated from B. amyloliquefaciens FZB24 according the method described previously (Pitcher et al., 1989, Letters in Applied Microbiology 8(4): 151-156). A 1984 bp fragment of the B. amyloliquefaciens FZB24 chromosome was amplified by PCR using primers 1202693 and 1203820 shown below.

Primer 1202693:
(SEQ ID NO: 97)
5′-GATCGGATCCATCGCCGTCCGCAAAACCGATATAA-3′
Primer 1203820:
(SEQ ID NO: 98)
5′-CGGAAGCATTTGGGAGATCTCGATCGCTTCAGCGTACGCG-3′

A cleavage site for restriction enzyme BamHI (bold) was incorporated into primer 1203820.

A second 1927 bp fragment of the B. amyloliquefaciens FZB24 chromosome was amplified by PCR using primers 1203819 and 1202694 shown below.

Primer 1203819:
(SEQ ID NO: 99)
5′-CGCGTACGCTGAAGCGATCGAGATCTOCCAAATGOTTCCG-3′
Primer 1202694:
(SEQ ID NO: 100)
5′-GATCGGATCCATTATATCGCCAGGCAGGACGGTGATGACATCTCC
AAC-3′

A cleavage site for the BamHI restriction enzyme (bold) was incorporated into primer 1202694. Primers 1203820 and 1203819 are complementary.

The respective DNA fragments were amplified by PCR using an Expand® High Fidelity^{Plus} PCR System (Roche Diagnostics, Mannheim, Germany). The PCR mixture contained approximately 1 μg of B. amyloliquefaciens FZB24 genomic DNA, 1 μl of sense primer (50 pmol/μl), 1 μl of anti-sense primer (50 pmol/μl), 10 μl of 5×PCR buffer with 15 mM MgCl₂, 1 μl of dNTP mix (10 mM each), 32.25 μl of water, and 0.75 μl of DNA polymerase mix (3.5 U/μl). An Eppendorf® Mastercycler® thermocycler was used to amplify the fragment with the following settings: One cycle at 94° C. for 2 minutes; 10 cycles each at 94° C. for 15 seconds, 58° C. for 30 seconds, and 72° C. for 2 minutes; 15 cycles each at 94° C. for 15 seconds, 58° C. for 30 seconds, and 72° C. for 2 minutes plus 5 second elongation at each successive cycle; one cycle at 72° C. for 7 minutes; and a 4° C. hold. The PCR products were purified from a 0.7% agarose (Amresco, Solon, Ohio) gel with 1×TBE buffer (10.8 g/l Tris Base, 5.5 g/l boric acid, and 2 mM EDTA pH 8) using a QIAquick® Gel Extraction Kit (Qiagen, Inc., Valencia, Calif., USA) according to the manufacturer's instructions.

The purified PCR products were used to create a single fragment by splice overlapping (SOE) PCR using an Expand® High Fidelity^{plus} PCR System as follows. The PCR mixture contained approximately 50 ng of gel purified PCR product from primer combination 1202693/1203820, approximately 50 ng of gel purified PCR product from primer combination 1203819/1202694, 1 μl of primer 1202693 (50 pmol/μl), 1 μl of primer 1202694 (50 pmol/μl), 10 μl of 5×PCR buffer with 15 mM MgCl₂, 1 μl of dNTP mix (10 mM each), 32.25 μl of water, and 0.75 μl of DNA polymerase mix (3.5 U/μl). An Eppendorf® Mastercycler® thermocycler was used to amplify the fragment with the following settings: One cycle at 94° C. for 2 minutes; 10 cycles each at 94° C. for 15 seconds, 58° C. for 30 seconds, and 72° C. for 4 minutes; 15 cycles each at 94° C. for 15 seconds, 58° C. for 30 seconds, and 72° C. for 4 minutes plus 5 second elongation at each successive cycle; one cycle at 72° C. for 7 minutes; and a 4° C. hold. The resulting 3959 bp PCR product was purified from a 0.7% agarose (Amresco) gel with 1×TBE buffer using a QIAquick® Gel Extraction Kit according to the manufacturer's instructions.

The purified PCR product and plasmid pShV2 (EP 0941349 B1) were each digested with BamHI and the resulting fragments were isolated by 0.7% agarose gel electrophoresis using TBE buffer followed by purification using a QIAquick® Gel Extraction Kit according to the manufacturer's instructions. The fragments were ligated using a Rapid DNA Ligation Kit following the manufacturer's instructions. A 2 μl aliquot of the ligation was used to transform E. coli OneShot™ cells according to the manufacturer's instructions. Plasmid DNA was prepared from E. coli transformants and digested using restriction enzyme BamHI followed by 0.7% agarose gel electrophoresis using TBE buffer. The plasmid identified as having the correct restriction pattern was designated pBM340.

The temperature-sensitive plasmid pBM340 was incorporated into the genome of B. amyloliquefaciens FZB24 by chromosomal integration and excision according to the method described previously (U.S. Pat. No. 5,843,720). B. amyloliquefaciens FZB24 transformants containing plasmid pBM340 were grown on TBAB selective medium at 50° C. to force integration of the vector. Desired integrants were chosen based on their ability to grow on TBAB erythromycin/lincomycin selective medium at 50° C. Integrants were then grown without selection in LB liquid medium at 37° C. to allow excision of the integrated plasmid. Cells were spread onto LB agar plates and screened for erythromycin-sensitivity.

Genomic DNA was prepared from several erythromycin/lincomycin sensitive isolates above accordingly to the method described previously (Pitcher et al., 1989, supra). Genomic PCR confirmed the disruption of the epsG and epsH coding sequences. The resulting strain was designated BaC0171 (FZB24 epsGΔ₈₈₇₋₁₀₄, epsHΔ₁₋₄₂₁).

### Example 2

### Construction of Transforming Plasmid, pBM331

Plasmid pBM331 was used for illustrative purposes to determine the increased transformation efficiency of strain BaC0171. This plasmid is a pNNB194 based plasmid (U.S. Pat. No. 5,958,728) which allows for selection of transformants, at 34° C., on agar plates containing erythromycin/lincomycin. Plasmid, pBM331, was constructed as follows.

Plasmid pBM331 was designed to delete the B. amyloliquefaciens srfAC gene. Genomic DNA was isolated from B. amyloliquefaciens FZB24 according the method described previously (Pitcher et al., 1989, supra). A 532 bp fragment of the B. amyloliquefaciens FZB24 chromosome was amplified by PCR using primers 1202649 and 1202650 shown below.

Primer 1202649:
(SEQ ID NO: 101)
5′-GATCCTCGAGAAAACGGTAAAAGAGACG-3′
Primer 1202650:
(SEQ ID NO: 102)
5′-CCATTGGCGGGCTTCCTCCTTTTCTGCTCCGCTCCCCCCTTCTG
TT-3′

A cleavage site for restriction enzyme XhoI (bold) was incorporated into primer 1202649.

A second 533 bp fragment of the B. amyloliquefaciens FZB24 chromosome was amplified by PCR using primers 1203819 and 1202694 shown below.

Primer 1202651:
(SEQ ID NO: 103)
5′-AACAGAAGGGGGGAGCGGAGCAGAAAAGGAGGAAGCCCGCCAAT
GG-3′
Primer 1202652:
(SEQ ID NO: 104)
5′-GATCCTCGAGTGAAAGAAGGCAGG-3′

A cleavage site for the BamHI restriction enzyme (bold) was incorporated into primer 1202694. Primers 1202650 and 1202651 are complementary.

The respective DNA fragments were amplified by PCR using an Expand® High Fidelity^{plus} PCR System. The PCR mixture contained approximately 1 μg of B. amyloliquefaciens FZB24 genomic DNA, 1 μl of sense primer (50 pmol/μl), 1 μl of anti-sense primer (50 pmol/μl), 10 μl of 5×PCR buffer with 15 mM MgCl₂, 1 μl of dNTP mix (10 mM each), 32.25 μl of water, and 0.75 μl of DNA polymerase mix (3.5 U/μl). An Eppendorf® Mastercycler® thermocycler was used to amplify the fragment with the following settings: One cycle at 94° C. for 2 minutes; 10 cycles each at 94° C. for 15 seconds, 58° C. for 30 seconds, and 72° C. for 30 seconds; 15 cycles each at 94° C. for 15 seconds, 58° C. for 30 seconds, and 72° C. for 30 seconds plus 5 second elongation at each successive cycle; one cycle at 72° C. for 7 minutes; and a 4° C. hold. The PCR products were purified from a 0.7% agarose (Amresco, Solon, Ohio, USA) gel with 1×TBE buffer using a QIAquick® Gel Extraction Kit according to the manufacturer's instructions.

The purified PCR products were used to create a single fragment b7 SOE PCR using an Expand® High Fidelity^{plus} PCR System as follows. The PCR mixture contained approximately 50 ng of gel purified PCR product from primer combination 1202649/1202650, approximately 50 ng of gel purified PCR product from primer combination 1202651/1202652, 1 μl of primer 1202649 (50 pmol/μl), 1 μl of primer 1202652 (50 pmol/μl), 10 μl of 5×PCR buffer with 15 mM MgCl₂, 1 μl of dNTP mix (10 mM each), 32.25 μl of water, and 0.75 μl of DNA polymerase mix (3.5 U/μl). An Eppendorf® Mastercycler® thermocycler was used to amplify the fragment with the following settings: One cycle at 94° C. for 2 minutes; 10 cycles each at 94° C. for 15 seconds, 58° C. for 30 seconds, and 72° C. for 1 minute; 15 cycles each at 94° C. for 15 seconds, 58° C. for 30 seconds, and 72° C. for 1 minute plus 5 second elongation at each successive cycle; one cycle at 72° C. for 7 minutes; and a 4° C. hold. The resulting 1019 bp PCR product was purified from a 0.7% agarose (Amresco) gel with 1×TBE buffer using a QIAquick® Gel Extraction Kit according to the manufacturer's instructions.

The purified PCR product and plasmid pBM317 (WO 2014/052630) were each digested with XhoI and the resulting fragments were isolated by 0.7% agarose gel electrophoresis using TBE buffer followed by purification using a QIAquick® Gel Extraction Kit according to the manufacturer's instructions. The purified fragments were ligated using a Rapid DNA Ligation Kit following the manufacturer's instructions. A 2 μl aliquot of the ligation was used to transform E. coli OneShot™ cells according to the manufacturer's instructions. Plasmid DNA was prepared from E. coli transformants and digested using restriction enzyme XhoI followed by 0.7% agarose gel electrophoresis using TBE buffer. The plasmid identified as having the correct restriction pattern was designated pBM331. Prior to transformation experiments, plasmid pBM331 was isolated from E. coli SCS110 (Stratagene, La Jolla, Calif., USA) cells using the Qiagen® Maxi-prep procedure according to the manufacturer's instructions (Qiagen, Inc., Valencia, Calif., USA).

### Example 3

### Transformation Efficiency of a B. amyloliquefaciens Strain Comprising a Disrupted epsA-O Operon (BaC0171)

The B. amyloliquefaciens strain BaC0171 comprising a disruption of the epsA-O operon described in Example 1 was spread onto LB agar plates to obtain single colony isolates after incubation at 37° C. overnight. After overnight incubation, one colony was used to inoculate 10 ml of LB liquid medium, and grown in a shaking incubator at 37° C. overnight. Approximately 250 μl of the overnight culture was used to inoculate 12 ml of Spizizen I medium containing 30 μl of 1 M MgCl₂. Growth was monitored using a Klett densitometer until cells entered early stationary phase. The cells were harvested, and 500 ml of the cell culture was added to a 15 ml Falcon 2059 tube. Transforming plasmid DNA (plasmid pBM331 bearing the erythromycin resistance gene described in Example 2) was added to the transformation mixture in the amounts indicated below and incubated at 34° C., 250 rpm for 30 minutes. After 30 minutes, 2 microliters of 50 mg/ml erythromycin was added to the transformation mixture. The culture was further incubated at 250 rpm, 34° C. for an additional hour, after which cells were spread onto TBAB plus erythromycin agar plates. The plates were incubated in a 34° C. incubator until colonies appeared. Colonies were counted the following day to determine transformation efficiency.

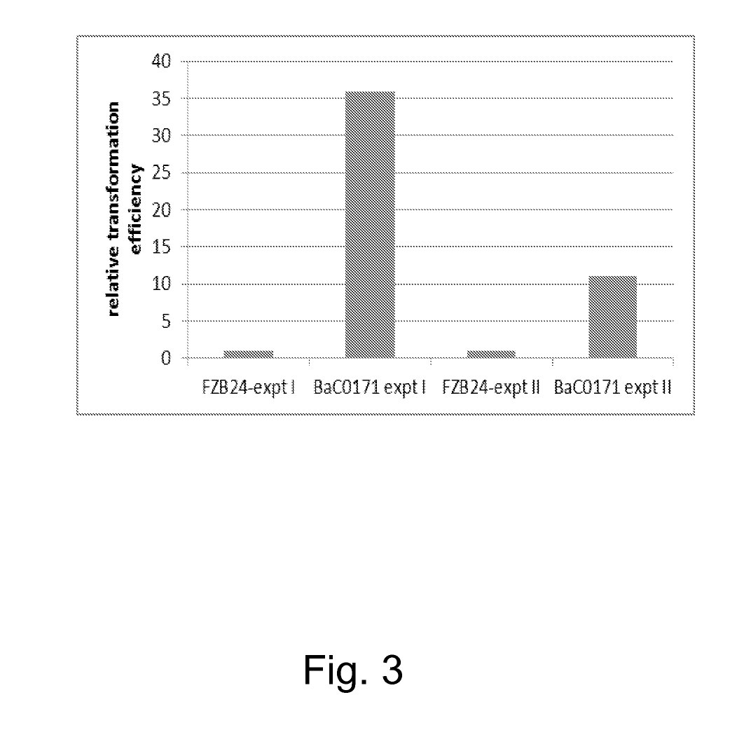

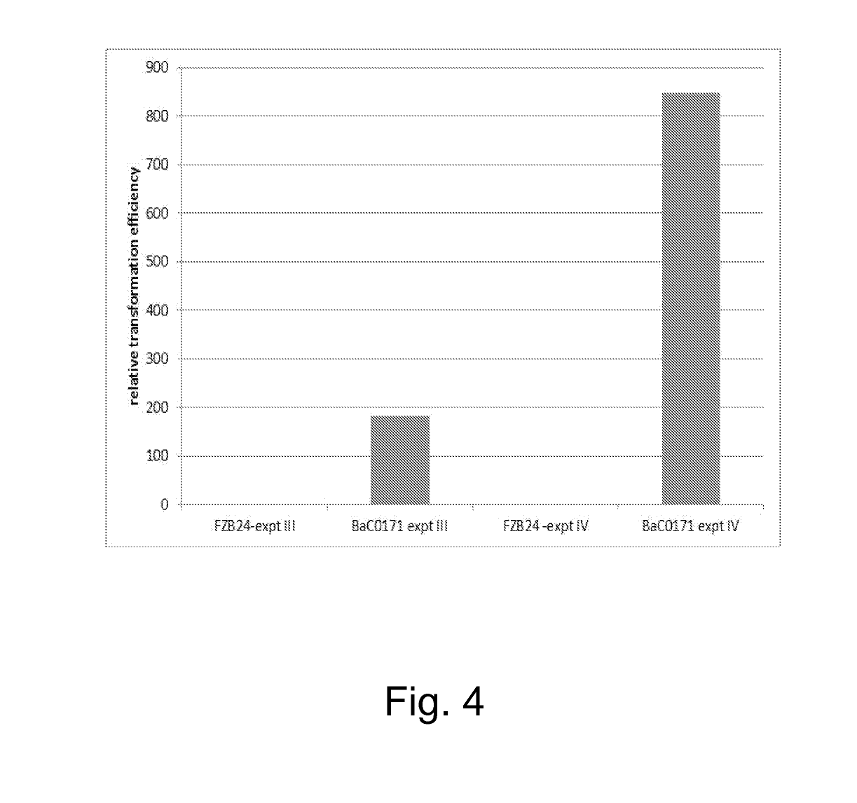

The results of two independent experiments using two micrograms of transforming plasmid DNA are shown in FIG. 3 (Actual transformation frequencies were as follows: Expt I FZB24 15 CFUs, BaC0171 541 CFUs. Expt II FZB24 2 CFUs, BaC0171 22 CFUs). The results of two additional independent experiments using four micrograms of transforming plasmid DNA are shown in FIG. 4 (Actual transformation frequencies were as follows: Expt I FZB24 12 CFUs, BaC0171 290 CFUs. Expt II FZB24 7 CFUs, BaC0171>5900 CFUs). Based on these experiments, disruption of the epsA-O operon results in a Bacillus strain (strain BaC0171) showing significantly higher transformation efficiency when compared to parent strain FZB24.

### Example 4

### Construction of a B. licheniformis mecA-Disrupted Strain (TaHy9)

Plasmid pBM294 was designed to delete 500 bp within the B. licheniformis mecA gene. Genomic DNA was isolated from B. licheniformis SJ1904 according to the method described previously (Pitcher et al., 1989, supra). A 323 bp fragment of the B. licheniformis SJ1904 chromosome, including the first 67 bp of the mecA coding sequence, was amplified by PCR using primers 0612056 and 0612057 shown below.

Primer 0612056 (SEQ ID NO: 105):
5′-GAATTCCATTAATAGCTGCTG-3′
Primer 0612057 (SEQ ID NO: 106):
5′-TCCATACTCTTTCAGCATGGTCTTCGATATCACCGT-3′

A cleavage site for restriction enzyme EcoRI (bold) was incorporated into primer 0612056. Primer 0612057 incorporates 18 bp (underlined) corresponding to by 568 to 588 of the mecA coding sequence.

A second 288 bp fragment of the B. licheniformis SJ1904 chromosome, including the segment from nucleotides 568 to 639 of the mecA coding sequence, was amplified by PCR using primers 0612058 and 0612060 shown below.

Primer 0612058 (SEQ ID NO: 107):
5′-ACGGTGATATCGAAGACCATGCTGAAAGAGTATGGA-3′
Primer 0612060 (SEQ ID NO: 108):
5′-CTCGAGCGCATCCTOCCAAAATC-3′

A cleavage site for the XhoI restriction enzyme (bold) was incorporated into primer 0612060. Primer 0612058 incorporates 18 bp (underlined) corresponding to by 47 to 67 of the mecA coding sequence. Primers 0612057 and 0612058 are complementary.

The respective DNA fragments were amplified by PCR using an Expand® High Fidelity^{plus} PCR System. The PCR mixture contained 4 μl (˜1 μg) of B. licheniformis SJ1904 genomic DNA, 1 μl of sense primer (50 pmol/μl), 1 μl of anti-sense primer (50 pmol/μl), 10 μl of 5×PCR buffer with 15 mM MgCl₂, 1 μl of dNTP mix (10 mM each), 32.25 μl of water, and 0.75 μl of DNA polymerase mix (3.5 U/μl). An Eppendorf® Mastercycler® thermocycler was used to amplify the fragment with the following settings: One cycle at 94° C. for 2 minutes; 10 cycles each at 94° C. for 15 seconds, 58° C. for 30 seconds, and 72° C. for 20 seconds; 15 cycles each at 94° C. for 15 seconds, 58° C. for 30 seconds, and 72° C. for 20 seconds plus 5 second elongation at each successive cycle; one cycle at 72° C. for 7 minutes; and a 4° C. hold. The PCR products were purified from a 1.2% agarose (Amresco, Solon, Ohio, USA) gel with 1×TBE buffer using a QIAquick® Gel Extraction Kit according to the manufacturer's instructions.

The purified PCR products were used in a subsequent PCR reaction to create a single fragment by SOE PCR (using an Expand® High Fidelity^{plus} PCR System as follows. The PCR mixture contained 2 μl (˜50 ng) of gel purified PCR product from primer combination 0612056/0612057, 2 μl (˜50 ng) of gel purified PCR product from primer combination 0612058/0612060, 1 μl of primer 0612056 (50 pmol/μl), 1 μl of primer 0612060 (50 pmol/μl), 10 μl of 5×PCR buffer with 15 mM MgCl₂, 1 μl of dNTP mix (10 mM each), 32.25 μl of water, and 0.75 μl of DNA polymerase mix (3.5 U/μl). An Eppendorf® Mastercycler® thermocycler was used to amplify the fragment with the following settings: One cycle at 94° C. for 2 minutes; 10 cycles each at 94° C. for 15 seconds, 58° C. for 30 seconds, and 72° C. for 40 seconds; 15 cycles each at 94° C. for 15 seconds, 58° C. for 30 seconds, and 72° C. for 40 seconds plus 5 second elongation at each successive cycle; one cycle at 72° C. for 7 minutes; and a 4° C. hold. The resulting 611 bp PCR product was purified from a 1.2% agarose (Amresco) gel with 1×TBE buffer using a QIAquick® Gel Extraction Kit according to the manufacturer's instructions.

The purified PCR product was cloned into plasmid pCR2.1-TOPO (Invitrogen, Carlsbad, Calif., USA) according to the manufacturer's instructions, resulting in a plasmid designated pBM293. Plasmid pBM293 and plasmid pNNB194 (U.S. Pat. No. 5,958,728) were digested with restriction enzymes XhoI and EcoRI to isolate the 606 bp insert fragment and vector fragment, respectively. These fragments were isolated by 1% agarose gel electrophoresis using TBE buffer followed by purification using a QIAquick® Gel Extraction Kit according to the manufacturer's instructions. The fragments were ligated using a Rapid DNA Ligation Kit following the manufacturer's instructions. A 2 μl aliquot of the ligation was used to transform E. coli Sure™ cells according to the manufacturer's instructions. Plasmid DNA was prepared from E. coli transformants and digested using restriction enzymes EcoRI and XhoI, followed by 0.7% agarose gel electrophoresis using TBE buffer, and the plasmid identified as having the correct restriction pattern was designated pBM294.

The temperature-sensitive plasmid pBM294 was incorporated into the genome of B. licheniformis SJ1904 by chromosomal integration and excision according to the method described previously (U.S. Pat. No. 5,843,720). B. licheniformis SJ1904 transformants containing plasmid pBM294 were grown on TBAB selective medium at 50° C. to force integration of the vector. Desired integrants were chosen based on their ability to grow on TBAB erythromycin/lincomycin selective medium at 50° C. Integrants were then grown without selection in LB medium at 37° C. to allow excision of the integrated plasmid. Cells were spread onto LB agar plates and screened for erythromycin-sensitivity.

Genomic DNA was prepared from several erythromycin/lincomycin sensitive isolates above accordingly to the method previously described (Pitcher et al., 1989, supra). Genomic PCR confirmed disruption of mecA and the resulting strain was designated TaHY9.

### Example 5

### Transformation Efficiency of a B. licheniformis mecA-Disrupted Strain (TaHy9)

The B. licheniformis mecA-disrupted strain TaHy9 from Example 4 was spread onto LB agar plates to obtain confluent growth after incubation at 37° C. overnight. After overnight incubation, approximately 2-3 ml of Spizizen I medium was added to each plate. Cells were scraped using sterile spreaders and transferred into 15 ml Falcon 2059 tubes. Approximately 500 μl of this culture was used to inoculate 50 ml of Spizizen I medium containing 1% xylose as the sole carbon source. Growth was monitored using a Klett densitometer. At each cell density corresponding to Klett unit 140, 160, 180, 200, and 250 μl of the culture plus 250 μl Spizizen II medium containing 2 mM EGTA was added to a Falcon 2059 tube. One microgram of transforming DNA (B. licheniformis MDT232 chromosomal DNA containing a spectinomycin resistance expression cassette integrated at the glpD locus; see WO 2008/079895) was added to each tube. Two microliters of 50 μg/ml spectinomycin was also included in the transformation mix. Tubes were incubated at 37° C. on a rotational shaker set at 250 rpm for 1 hour. Transformation reactions were plated to LB agar plates containing 120 μg/ml of spectinomycin. Colonies were counted the following day to determine transformation efficiency.

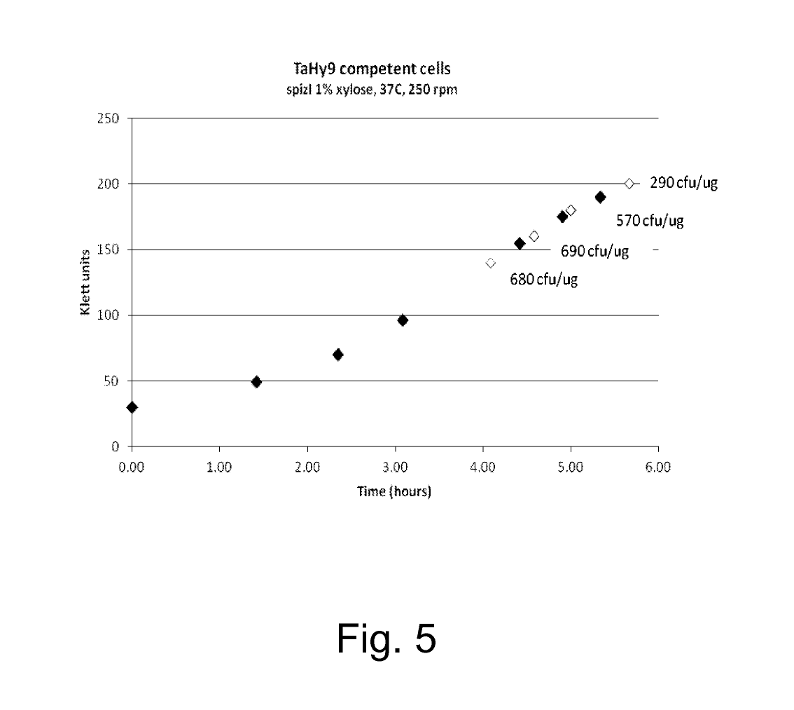

A B. licheniformis competent state in a mecA disrupted strain was reached during the exponential growth phase and declined as cells entered stationary phase (FIG. 5). The highest transformation efficiency was obtained at a Klett densitometer reading of 160 and transformation efficiency declined as cells reached stationary phase.

### Example 6

### DNA Microarray Analysis of a B. licheniformis mecA-Disrupted Strain (TaHy9)

In order to obtain additional understanding of B. licheniformis competence development, DNA microarray technology was used to compare global transcription profiles in the B. licheniformis strains TaHy9 (supra) and MMar2 (B. licheniformis SJ1904 amyL:Xylp-comK, a B. licheniformis strain containing a second copy of the comK gene under transcriptional control of the xylose inducible promoter; see US 2010/0028944). Strains TaHy9 and MMar2 were grown in triplicate shake flask cultures containing Spizizen I+1% xylose medium to 160 reading on a Klett densitometer as described in Example 2. RNA samples were purified from triplicate shake flask cultures and DNA microarray analysis was conducted using custom Affymetrix microarray chips (Affymetrix Inc., Santa Clara, Calif., USA) designed for use with B. subtilis strains 168, A164 and B. licheniformis SJ1904. RNA was isolated using a FastRNA™ Pro Blue Kit (MP Biomedicals, LLC, Solon, Ohio, USA), according to the manufacturer's recommendations for bacterial RNA isolation. Cells were disrupted two times for 40 seconds at maximum speed (speed 6) in the FastPrep® Instrument (MP Biomedicals, LLC, Solon, Ohio, USA). In addition, 45 μg of RNA from each sample were further purified using in an RNeasy™ column (Qiagen, Inc., Valencia, Calif., USA) according to the manufacturer's specifications. The quality, integrity and concentrations of the RNA samples were measured using a Bioanalyzer instrument (Agilent Technologies Inc., Santa Clara, Calif., USA). RNA samples and custom Affymetrix microarray chips were submitted to the UCLA Clinical Microarray Core Research Facility (Los Angeles, Calif., USA) for labeling, hybridization and scanning. Microarray data were analyzed using a custom script as described previously (Gillespie et al., 2010, BMC Research Notes 3(81)). Data were normalized using Robust Multi-array Average (RMA) (Irizarry et al., 2003, Biostatistics 4: 249-264) followed by differential expression analysis using Linear Models for Microarray Data (Limma) software (Smyth, 2005, Limma: Linear models for microarray data. In: Bioinformatics and Computational Biology Solutions using R and Bioconductor, Springer, New York, pp. 397-420).

The microarray data revealed significantly increased transcript levels for epsH, tapA, sigW and tasA genes in Bacillus licheniformis strain TaHy9 compared to strain MMar2 (p<0.05).

The invention is further defined in the following paragraphs:

1. A mutant Bacillus strain, comprising a disruption of an endogenous epsA-O operon.
2. The mutant Bacillus strain of paragraph 1, wherein the disruption of the endogenous epsA-O operon is a disruption of an endogenous epsA coding sequence.
3. The mutant Bacillus strain of paragraph 2, wherein the endogenous epsA coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 46, 47, or 48; (b) hybridizes under at least low, medium, medium-high, high, or very high stringency conditions with the full-length complementary strand of SEQ ID NO: 1, 2, or 3; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 1, 2, or 3.
4. The mutant Bacillus strain of paragraph 2 or 3, wherein the disruption of the endogenous epsA-O operon is in a coding region of the endogenous epsA coding sequence.
5. The mutant Bacillus strain of any of paragraphs 2-4, wherein the disruption of the endogenous epsA-O operon is in a control sequence of the endogenous epsA coding sequence.
6. The mutant Bacillus strain of any of paragraphs 1-5, wherein the disruption of the endogenous epsA-O operon is a disruption of an endogenous epsB coding sequence.
7. The mutant Bacillus strain of paragraph 6, wherein the endogenous epsB coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 49, 50, or 51; (b) hybridizes under at least low, medium, medium-high, high, or very high stringency conditions with the full-length complementary strand of SEQ ID NO: 4, 5, or 6; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 4, 5, or 6.
8. The mutant Bacillus strain of paragraph 6 or 7, wherein the disruption of the endogenous epsA-O operon is in a coding region of the endogenous epsB coding sequence.
9. The mutant Bacillus strain of any of paragraphs 6-8, wherein the disruption of the endogenous epsA-O operon is in a control sequence of the endogenous epsB coding sequence.
10. The mutant Bacillus strain of any of paragraphs 1-9, wherein the disruption of the endogenous epsA-O operon is a disruption of an endogenous epsC coding sequence.
11. The mutant Bacillus strain of paragraph 10, wherein the endogenous epsC coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 52, 53, or 54; (b) hybridizes under at least low, medium, medium-high, high, or very high stringency conditions with the full-length complementary strand of SEQ ID NO: 7, 8, or 9; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 7, 8, or 9.
12. The mutant Bacillus strain of paragraph 10 or 11, wherein the disruption of the endogenous epsA-O operon is in a coding region of the endogenous epsC coding sequence.
13. The mutant Bacillus strain of any of paragraphs 10-12, wherein the disruption of the endogenous epsA-O operon is in a control sequence of the endogenous epsC coding sequence.
14. The mutant Bacillus strain of any of paragraphs 1-13, wherein the disruption of the endogenous epsA-O operon is a disruption of an endogenous epsD coding sequence.
15. The mutant Bacillus strain of paragraph 14, wherein the endogenous epsD coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 55, 56, or 57; (b) hybridizes under at least low, medium, medium-high, high, or very high stringency conditions with the full-length complementary strand of SEQ ID NO: 10, 11, or 12; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 10, 11, or 12.
16. The mutant Bacillus strain of paragraph 14 or 15, wherein the disruption of the endogenous epsA-O operon is in a coding region of the endogenous epsD coding sequence.
17. The mutant Bacillus strain of any of paragraphs 14-16, wherein the disruption of the endogenous epsA-O operon is in a control sequence of the endogenous epsD coding sequence.
18. The mutant Bacillus strain of any of paragraphs 1-17, wherein the disruption of the endogenous epsA-O operon is a disruption of an endogenous epsE coding sequence.
19. The mutant Bacillus strain of paragraph 18, wherein the endogenous epsE coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 58, 59, or 60; (b) hybridizes under at least low, medium, medium-high, high, or very high stringency conditions with the full-length complementary strand of SEQ ID NO: 10, 11, or 12; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 10, 11, or 12.
20. The mutant Bacillus strain of paragraph 18 or 19, wherein the disruption of the endogenous epsA-O operon is in a coding region of the endogenous epsE coding sequence.
21. The mutant Bacillus strain of any of paragraphs 18-20, wherein the disruption of the endogenous epsA-O operon is in a control sequence of the endogenous epsE coding sequence.
22. The mutant Bacillus strain of any of paragraphs 1-21, wherein the disruption of the endogenous epsA-O operon is a disruption of an endogenous epsF coding sequence.
23. The mutant Bacillus strain of paragraph 22, wherein the endogenous epsF coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 61, 62, or 63; (b) hybridizes under at least low, medium, medium-high, high, or very high stringency conditions with the full-length complementary strand of SEQ ID NO: 16, 17, or 18; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 16, 17, or 18.
24. The mutant Bacillus strain of paragraph 22 or 23, wherein the disruption of the endogenous epsA-O operon is in a coding region of the endogenous epsF coding sequence.
25. The mutant Bacillus strain of any of paragraphs 22-24, wherein the disruption of the endogenous epsA-O operon is in a control sequence of the endogenous epsF coding sequence.
26. The mutant Bacillus strain of any of paragraphs 1-25, wherein the disruption of the endogenous epsA-O operon is a disruption of an endogenous epsG coding sequence.
27. The mutant Bacillus strain of paragraph 26, wherein the endogenous epsG coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 64, 65, or 66; (b) hybridizes under at least low, medium, medium-high, high, or very high stringency conditions with the full-length complementary strand of SEQ ID NO: 19, 20, or 21; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 19, 20, or
28. The mutant Bacillus strain of paragraph 26 or 27, wherein the disruption of the endogenous epsA-O operon is in a coding region of the endogenous epsG coding sequence.
29. The mutant Bacillus strain of any of paragraphs 26-28, wherein the disruption of the endogenous epsA-O operon is in a control sequence of the endogenous epsG coding sequence.
30. The mutant Bacillus strain of any of paragraphs 1-29, wherein the disruption of the endogenous epsA-O operon is a disruption of an endogenous epsH coding sequence.
31. The mutant Bacillus strain of paragraph 30, wherein the endogenous epsH coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 67, 68, or 69; (b) hybridizes under at least low, medium, medium-high, high, or very high stringency conditions with the full-length complementary strand of SEQ ID NO: 22, 23, or 24; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 22, 23, or 24.
32. The mutant Bacillus strain of paragraph 30 or 31, wherein the disruption of the endogenous epsA-O operon is in a coding region of the endogenous epsH coding sequence.
33. The mutant Bacillus strain of any of paragraphs 30-32, wherein the disruption of the endogenous epsA-O operon is in a control sequence of the endogenous epsH coding sequence.
34. The mutant Bacillus strain of any of paragraphs 1-33, wherein the disruption of the endogenous epsA-O operon is a disruption of an endogenous epsI coding sequence.
35. The mutant Bacillus strain of paragraph 34, wherein the endogenous epsI coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 70, 71, or 72; (b) hybridizes under at least low, medium, medium-high, high, or very high stringency conditions with the full-length complementary strand of SEQ ID NO: 25, 26, or 27; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 25, 26, or 27.
36. The mutant Bacillus strain of paragraph 34 or 35, wherein the disruption of the endogenous epsA-O operon is in a coding region of the endogenous epsI coding sequence.
37. The mutant Bacillus strain of any of paragraphs 34-36, wherein the disruption of the endogenous epsA-O operon is in a control sequence of the endogenous epsI coding sequence.
38. The mutant Bacillus strain of any of paragraphs 1-37, wherein the disruption of the endogenous epsA-O operon is a disruption of an endogenous epsJ coding sequence.
39. The mutant Bacillus strain of paragraph 38, wherein the endogenous epsJ coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 73, 74, or 75; (b) hybridizes under at least low, medium, medium-high, high, or very high stringency conditions with the full-length complementary strand of SEQ ID NO: 28, 29, or 30; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 28, 29, or 30.
40. The mutant Bacillus strain of paragraph 38 or 39, wherein the disruption of the endogenous epsA-O operon is in a coding region of the endogenous epsJ coding sequence.
41. The mutant Bacillus strain of any of paragraphs 38-40, wherein the disruption of the endogenous epsA-O operon is in a control sequence of the endogenous epsJ coding sequence.
42. The mutant Bacillus strain of any of paragraphs 1-41, wherein the disruption of the endogenous epsA-O operon is a disruption of an endogenous epsK coding sequence.
43. The mutant Bacillus strain of paragraph 42, wherein the endogenous epsK coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 76, 77, or 78; (b) hybridizes under at least low, medium, medium-high, high, or very high stringency conditions with the full-length complementary strand of SEQ ID NO: 31, 32, or 33; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 31, 32, or 33.
44. The mutant Bacillus strain of paragraph 42 or 43, wherein the disruption of the endogenous epsA-O operon is in a coding region of the endogenous epsK coding sequence.
45. The mutant Bacillus strain of any of paragraph 42-44, wherein the disruption of the endogenous epsA-O operon is in a control sequence of the endogenous epsK coding sequence.
46. The mutant Bacillus strain of any of paragraphs 1-45, wherein the disruption of the endogenous epsA-O operon is a disruption of an endogenous epsL coding sequence.
47. The mutant Bacillus strain of paragraph 46, wherein the endogenous epsL coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 79, 80, or 81; (b) hybridizes under at least low, medium, medium-high, high, or very high stringency conditions with the full-length complementary strand of SEQ ID NO: 34, 35, or 36; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 34, 35, or 36.
48. The mutant Bacillus strain of paragraph 46 or 47, wherein the disruption of the endogenous epsA-O operon is in a coding region of the endogenous epsL coding sequence.
49. The mutant Bacillus strain of any of paragraphs 46-48, wherein the disruption of the endogenous epsA-O operon is in a control sequence of the endogenous epsL coding sequence.
50. The mutant Bacillus strain of any of paragraphs 1-49, wherein the disruption of the endogenous epsA-O operon is a disruption of an endogenous epsM coding sequence.
51. The mutant Bacillus strain of paragraph 50, wherein the endogenous epsM coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 82, 83, or 84; (b) hybridizes under at least low, medium, medium-high, high, or very high stringency conditions with the full-length complementary strand of SEQ ID NO: 37, 38, or 39; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 37, 38, or 39.
52. The mutant Bacillus strain of paragraph 50 or 51, wherein the disruption of the endogenous epsA-O operon is in a coding region of the endogenous epsM coding sequence.
53. The mutant Bacillus strain of any of paragraphs 50-52, wherein the disruption of the endogenous epsA-O operon is in a control sequence of the endogenous epsM coding sequence.
54. The mutant Bacillus strain of any of paragraphs 1-53, wherein the disruption of the endogenous epsA-O operon is a disruption of an endogenous epsN coding sequence.
55. The mutant Bacillus strain of paragraph 54, wherein the endogenous epsN coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 85, 86, or 87; (b) hybridizes under at least low, medium, medium-high, high, or very high stringency conditions with the full-length complementary strand of SEQ ID NO: 40, 41, or 42; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 40, 41, or 42.
56. The mutant Bacillus strain of paragraph 54 or 55, wherein the disruption of the endogenous epsA-O operon is in a coding region of the endogenous epsN coding sequence.
57. The mutant Bacillus strain of any of paragraphs 54-56, wherein the disruption of the endogenous epsA-O operon is in a control sequence of the endogenous epsN coding sequence.
58. The mutant Bacillus strain of any of paragraphs 1-57, wherein the disruption of the endogenous epsA-O operon is a disruption of an endogenous epsO coding sequence.
59. The mutant Bacillus strain of paragraph 58, wherein the endogenous epsO coding sequence (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 88, 89, or 90; (b) hybridizes under at least low, medium, medium-high, high, or very high stringency conditions with the full-length complementary strand of SEQ ID NO: 43, 44, or 45; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 43, 44, or 45.
60. The mutant Bacillus strain of paragraph 58 or 59, wherein the disruption of the endogenous epsA-O operon is in a coding region of the endogenous epsO coding sequence.
61. The mutant Bacillus strain of any of paragraphs 58-60, wherein the disruption of the endogenous epsA-O operon is in a control sequence of the endogenous epsO coding sequence.
62. The mutant Bacillus strain of any of paragraphs 1-61, wherein the endogenous epsA-O operon (a) encodes for at least one polypeptide having at least 60%, e.g., at least 70%, at least 75%, at least 80%, at least 85%, at least 90%, at least 95%, at least 96%, at least 97%, at least 98%, at least 99%, or 100% sequence identity to any of SEQ ID NOs: 46-90; (b) comprises at least one coding sequence that hybridizes under at least low, medium, medium-high, high, or very high stringency conditions with the full-length complementary strand of any of SEQ ID NOs: 1-45; or (c) comprises at least one coding sequence that has at least 60%, e.g., at least 70%, at least 75%, at least 80%, at least 85%, at least 90%, at least 95%, at least 96%, at least 97%, at least 98%, at least 99%, or 100% sequence identity to any of SEQ ID NOs: 1-45.
63. The mutant Bacillus strain of any of paragraphs 1-62, wherein the endogenous epsA-O operon encodes for at least one polypeptide having at least 60%, e.g., at least 70%, at least 75%, at least 80%, at least 85%, at least 90%, at least 95%, at least 96%, at least 97%, at least 98%, at least 99%, or 100% sequence identity to any of SEQ ID NOs: 46-90.
64. The mutant Bacillus strain of any of paragraphs 1-63, wherein the endogenous epsA-O operon encodes for at least one polypeptide comprising or consisting of any of SEQ ID NOs: 46-90.
65. The mutant Bacillus strain of any of paragraphs 1-64, wherein the endogenous epsA-O operon comprises at least one coding sequence that hybridizes under at least low, medium, medium-high, high, or very high stringency conditions with the full-length complementary strand of any of SEQ ID NOs: 1-45.
66. The mutant Bacillus strain of any of paragraphs 1-65, wherein the endogenous epsA-O operon comprises at least one coding sequence that has at least 60%, e.g., at least 70%, at least 75%, at least 80%, at least 85%, at least 90%, at least 95%, at least 96%, at least 97%, at least 98%, at least 99%, or 100% sequence identity to any of SEQ ID NOs: 1-45.
67. The mutant Bacillus strain of any of paragraphs 1-66, wherein the endogenous epsA-O operon comprises at least one coding sequence comprising or consisting of any of SEQ ID NOs: 1-45.
68. The mutant Bacillus strain of any of paragraphs 1-67, wherein the disruption of the endogenous epsA-O operon occurs in the promoter sequence of the epsA-O operon.
69. The mutant Bacillus strain of any of paragraphs 1-68, wherein the disruption of the endogenous epsA-O operon occurs in one or more coding sequences of the epsA-O operon.
70. The mutant Bacillus strain of paragraph 69, wherein the disruption of the endogenous epsA-O operon occurs in:
(i) an epsA coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 46, 47, or 48; (b) hybridizes under at least low, medium, medium-high, high, or very high stringency conditions with the full-length complementary strand of SEQ ID NO: 1, 2, or 3; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 1, 2, or 3;
(ii) an epsB coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 49, 50, or 51; (b) hybridizes under at least low, medium, medium-high, high, or very high stringency conditions with the full-length complementary strand of SEQ ID NO: 4, 5, or 6; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 4, 5, or 6;
(iii) an epsC coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 52, 53, or 54; (b) hybridizes under at least low, medium, medium-high, high, or very high stringency conditions with the full-length complementary strand of SEQ ID NO: 7, 8, or 9; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 7, 8, or 9;
(iv) an epsD coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 55, 56, or 57; (b) hybridizes under at least low, medium, medium-high, high, or very high stringency conditions with the full-length complementary strand of SEQ ID NO: 10, 11, or 12; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 10, 11, or 12;
(v) an epsE coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 58, 59, or 60; (b) hybridizes under at least low, medium, medium-high, high, or very high stringency conditions with the full-length complementary strand of SEQ ID NO: 10, 11, or 12; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 10, 11, or 12;
(vi) an epsF coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 61, 62, or 63; (b) hybridizes under at least low, medium, medium-high, high, or very high stringency conditions with the full-length complementary strand of SEQ ID NO: 16, 17, or 18; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 16, 17, or 18;
(vii) an epsG coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 64, 65, or 66; (b) hybridizes under at least low, medium, medium-high, high, or very high stringency conditions with the full-length complementary strand of SEQ ID NO: 19, 20, or 21; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 19, 20, or 21;
(viii) an epsH coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 67, 68, or 69; (b) hybridizes under at least low, medium, medium-high, high, or very high stringency conditions with the full-length complementary strand of SEQ ID NO: 22, 23, or 24; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 22, 23, or 24;
(ix) an epsI coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 70, 71, or 72; (b) hybridizes under at least low, medium, medium-high, high, or very high stringency conditions with the full-length complementary strand of SEQ ID NO: 25, 26, or 27; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 25, 26, or 27;
(x) an epsJ coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 73, 74, or 75; (b) hybridizes under at least low, medium, medium-high, high, or very high stringency conditions with the full-length complementary strand of SEQ ID NO: 28, 29, or 30; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 28, 29, or 30;
(xi) an epsK coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 76, 77, or 78; (b) hybridizes under at least low, medium, medium-high, high, or very high stringency conditions with the full-length complementary strand of SEQ ID NO: 31, 32, or 33; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 31, 32, or 33;
(xii) an epsL coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 79, 80, or 81; (b) hybridizes under at least low, medium, medium-high, high, or very high stringency conditions with the full-length complementary strand of SEQ ID NO: 34, 35, or 36; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 34, 35, or 36;
(xiii) an epsM coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 82, 83, or 84; (b) hybridizes under at least low, medium, medium-high, high, or very high stringency conditions with the full-length complementary strand of SEQ ID NO: 37, 38, or 39; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 37, 38, or 39;
(xiv) an epsN coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 85, 86, or 87; (b) hybridizes under at least low, medium, medium-high, high, or very high stringency conditions with the full-length complementary strand of SEQ ID NO: 40, 41, or 42; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 40, 41, or 42; and/or
(xv) an epsO coding sequence that (a) encodes for a polypeptide having at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 88, 89, or 90; (b) hybridizes under at least low, medium, medium-high, high, or very high stringency conditions with the full-length complementary strand of SEQ ID NO: 43, 44, or 45; or (c) has at least 60%, e.g., at least 70%, at least 75%, at least 80%, at least 85%, at least 90%, at least 95%, at least 96%, at least 97%, at least 98%, at least 99%, or 100% sequence identity to SEQ ID NO: 43, 44, or 45.
   71. The mutant Bacillus strain of any of paragraphs 1-70, wherein the mutant produces at least 25% less (e.g., at least 50% less, at least 60% less, at least 70% less, at least 80% less, at least 90% less, or 100% less) of the polypeptide encoded by the endogenous epsA, epsB, epsC, epsD, epsE, epsF, epsG, epsH, epsI, epsJ, epsK, epsL, epsM, epsN, or epsO coding sequence compared to the parent Bacillus strain that lacks disruption of the endogenous epsA-O operon, when cultivated under identical conditions.
   72. The mutant Bacillus strain of any of paragraphs 1-71, wherein the endogenous epsA, epsB, epsC, epsD, epsE, epsF, epsG, epsH, epsI, epsJ, epsK, epsL, epsM, epsN, or epsO coding sequence is inactivated.
   73. The mutant Bacillus strain of any of paragraphs 1-72, wherein the disruption of the endogenous epsA-O operon comprises disruption of at least two (e.g., three, four, five, six, etc.) of the epsA-O operon coding sequences.
   74. The mutant Bacillus strain of any of paragraphs 1-73, wherein the disruption of the endogenous epsA-O operon comprises inactivation of at least two (e.g., three four, five, six, etc.) of the epsA-O operon coding sequences.
   75. The mutant Bacillus strain of any of paragraphs 1-74, wherein the disruption of the endogenous epsA-O operon comprises disruption of both the epsG and epsH coding sequences.
   76. The mutant Bacillus strain of any of paragraphs 1-74, wherein the disruption of the endogenous epsA-O operon comprises inactivation of both the epsG and epsH coding sequences.
   77. The mutant Bacillus strain of any of paragraphs 1-76, wherein the mutant has improved transformation efficiency compared to the parent Bacillus strain that lacks disruption of the endogenous epsA-O operon, when cultivated under identical conditions.
   78. The mutant Bacillus strain of any of paragraphs 1-77, wherein the mutant is capable of producing at least 10-fold (e.g., at least 100-fold, at least 1000-fold, at least 10000-fold, or at least 100000-fold) more transformants compared to the parent Bacillus strain that lacks disruption of the endogenous epsA-O operon, when cultivated under identical conditions.
   79. The mutant Bacillus strain of any of paragraphs 1-78, wherein the mutant further comprises disruption of an endogenous mecA gene.
   80. The mutant Bacillus strain of paragraph 79, wherein the disruption of the endogenous mecA gene occurs in the coding sequence and/or promoter sequence.
   81. The mutant Bacillus strain of paragraph 79 or 80, wherein the mutant produces at least 25% less (e.g., at least 50% less, at least 60% less, at least 70% less, at least 80% less, at least 90% less, or 100% less) of the polypeptide encoded by the endogenous mecA gene compared to the parent Bacillus strain that lacks disruption of the endogenous mecA gene, when cultivated under identical conditions.
   82. The mutant Bacillus strain of any of paragraphs 79-81, wherein the endogenous mecA gene is inactivated.
   83. The mutant Bacillus strain of any of paragraphs 79-82, wherein the mutant has improved transformation efficiency compared to the parent Bacillus strain that lacks disruption of the endogenous mecA gene, when cultivated under identical conditions.
   84. The mutant Bacillus strain of any of paragraphs 79-83, wherein the mutant is capable of producing at least 10-fold (e.g., at least 100-fold, at least 1000-fold, at least 10000-fold, or at least 100000-fold) more transformants compared to the parent Bacillus strain that lacks disruption of the endogenous mecA gene, when cultivated under identical conditions.
   85. The mutant Bacillus strain of any of paragraphs 1-84, wherein the parent Bacillus strain is selected from Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis.
   86. The mutant Bacillus strain of paragraph 85, wherein the parent Bacillus strain is a Bacillus amyloliquefaciens strain.
   87. The mutant Bacillus strain of paragraph 85, wherein the parent Bacillus strain is a Bacillus licheniformis strain.
   88. The mutant Bacillus strain of paragraph 85, wherein the parent Bacillus strain is a Bacillus subtilis strain.
   89. The mutant Bacillus strain of any of paragraphs 1-88, which further comprises a polynucleotide encoding a polypeptide.
   90. The mutant Bacillus strain of paragraph 89, wherein the polypeptide is native to the parent Bacillus strain.
   91. The mutant Bacillus strain of paragraph 89, wherein the polypeptide is heterologous to the parent Bacillus strain.
   92. The mutant Bacillus strain of any of paragraphs 89-91, wherein the polypeptide a hydrolase, isomerase, ligase, lyase, oxidoreductase, or transferase.
   93. The mutant Bacillus strain of paragraph 92, wherein the polypeptide is an alpha-glucosidase, aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, glucocerebrosidase, alpha-glucosidase, beta-glucosidase, invertase, laccase, lipase, mannosidase, mutanase, oxidase, pectinolytic enzyme, peroxidase, phospholipase, phytase, polyphenoloxidase, proteolytic enzyme, ribonuclease, transglutaminase, urokinase, or xylanase.
   94. The mutant Bacillus strain of any of paragraphs 89-91, wherein the polypeptide is an albumin, collagen, tropoelastin, elastin, or gelatin.
   95. The mutant Bacillus strain of any of paragraphs 1-88, which further comprises one or more polynucleotides encoding one or more polynucleotides of a fermentation pathway for producing a fermentation product.
   96. The mutant Bacillus strain of paragraph 95, wherein the one or more polypeptides are native to the parent Bacillus strain.
   97. The mutant Bacillus strain of paragraph 95 or 96, wherein the one or more polypeptides are heterologous to the parent Bacillus strain.
   98. The mutant Bacillus strain of any of paragraphs 95-97, wherein the fermentation product is an alcohol, an alkane, a cycloalkane, an alkene, an amino acid, a gas, an isoprenoid, a ketone, an organic acid, or a polyketide.
   99. A method for obtaining the Bacillus mutant strain of any of paragraphs 1-98, comprising
(a) disrupting an endogenous epsA-O operon in a parent Bacillus strain; and
(b) isolating the Bacillus mutant strain resulting from (a).
   100. A method of producing a polypeptide, comprising: cultivating a Bacillus mutant strain of any of paragraphs 89-94 under conditions conducive for producing the polypeptide.
   101. The method of paragraph 100, further comprising recovering the polypeptide.
   102. A method of producing a fermentation product, comprising cultivating a Bacillus mutant strain of any of paragraphs 95-98 under conditions conducive for producing the fermentation product.
   103. The method of paragraph 102, further comprising recovering the fermentation product.
   104. A method for obtaining a transformant of a Bacillus mutant strain of any of paragraphs 89-94, comprising
(a) introducing a polynucleotide encoding a polypeptide into a Bacillus mutant strain which comprises a disruption of an endogenous epsA-O operon; and
(b) isolating the Bacillus transformant.
   105. A method for obtaining a transformant of a Bacillus mutant strain of any of paragraphs 95-98, comprising
(a) introducing one or more polynucleotides encoding one or more polypeptides of a fermentation pathway for producing a fermentation product into a Bacillus mutant strain which comprises a disruption of an endogenous epsA-O operon; and
(b) isolating the Bacillus transformant.

Although the foregoing has been described in some detail by way of illustration and example for the purposes of clarity of understanding, it is apparent to those skilled in the art that any equivalent aspect or modification may be practiced. Therefore, the description and examples should not be construed as limiting the scope of the invention.

1. A mutant Bacillus strain, comprising a disruption of an endogenous epsA-O operon.

2. The mutant Bacillus strain of claim 1, wherein the endogenous epsA-O operon (a) encodes for at least one polypeptide having at least 60%, e.g., at least 70%, at least 75%, at least 80%, at least 85%, at least 90%, at least 95%, at least 96%, at least 97%, at least 98%, at least 99%, or 100% sequence identity to any of SEQ ID NOs: 46-90; (b) comprises at least one coding sequence that hybridizes under at least low, medium, medium-high, high, or very high stringency conditions with the full-length complementary strand of any of SEQ ID NOs: 1-45; or (c) comprises at least one coding sequence that has at least 60%, e.g., at least 70%, at least 75%, at least 80%, at least 85%, at least 90%, at least 95%, at least 96%, at least 97%, at least 98%, at least 99%, or 100% sequence identity to any of SEQ ID NOs: 1-45.

3. The mutant Bacillus strain of claim 1 or 2, wherein the mutant produces at least 25% less (e.g., at least 50% less, at least 60% less, at least 70% less, at least 80% less, at least 90% less, or 100% less) of the polypeptide encoded by the endogenous epsA, epsB, epsC, epsD, epsE, epsF, epsG, epsH, epsI, epsJ, epsK, epsL, epsM, epsN, or epsO coding sequence compared to the parent Bacillus strain that lacks disruption of the endogenous epsA-O operon, when cultivated under identical conditions.

4. The mutant Bacillus strain of claim 1, wherein the endogenous epsA, epsB, epsC, epsD, epsE, epsF, epsG, epsH, epsI, epsJ, epsK, epsL, epsM, epsN, or epsO coding sequence is inactivated.

5. The mutant Bacillus strain of claim 1, wherein disruption of the endogenous epsA-O operon comprises disruption of at least two (e.g., three, four, five, six, etc.) of the epsA-O operon coding sequences.

6. The mutant Bacillus strain of claim 1, wherein disruption of the endogenous epsA-O operon comprises inactivation of at least two (e.g., three four, five, six, etc.) of the epsA-O operon coding sequences.

7. The mutant Bacillus strain of claim 1, wherein the mutant has improved transformation efficiency compared to the parent Bacillus strain that lacks disruption of the endogenous epsA-O operon, when cultivated under identical conditions.

8. The mutant Bacillus strain of claim 1, wherein the mutant is capable of producing at least 10-fold (e.g., at least 100-fold, at least 1000-fold, at least 10000-fold, or at least 100000-fold) more transformants compared to the parent Bacillus strain that lacks disruption of the endogenous epsA-O operon, when cultivated under identical conditions.

9. The mutant Bacillus strain of claim 1, wherein the parent Bacillus strain is selected from Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis.

10. The mutant Bacillus strain of claim 1, which further comprises a polynucleotide encoding a polypeptide.

11. The mutant Bacillus strain of claim 1, which further comprises one or more polynucleotides encoding one or more polynucleotides of a fermentation pathway for producing a fermentation product.

12. A method for obtaining the Bacillus mutant strain of claim 1, comprising
(a) disrupting an endogenous epsA-O operon in a parent Bacillus strain; and
(b) isolating the Bacillus mutant strain resulting from (a).

13. A method of producing a polypeptide, comprising cultivating a Bacillus mutant strain of claim 10 under conditions conducive for producing the polypeptide.

14. The method of claim 13, further comprising recovering the polypeptide.

15. A method of producing a fermentation product, comprising cultivating a Bacillus mutant strain of claim 11 under conditions conducive for producing the fermentation product.

16. The method of claim 15, further comprising recovering the fermentation product.


SEQUENCE LISTING (108 sequences)

SEQ ID NO: 1 - DNA, 708 bp, Bacillus amyloliquefaciens
atgaatgagaatatgagttttaaagagttatttgacattattaaacacagatttttactgatttttatcatgacagcagttgtaacgctggtgacgggatacatccaattccgggtgatttcgcccgtttatcaggcatcgacccaagtgctcattcatgaaacaagcggtgaaaaaaattcgaatctcagcgacgttcagctgaatcttcattacaacaatacgtttcaaacgataatgaaaagcccggttgtgcttgagaaagtgaaacagaagctgcatctttctgagacggcatccgctttaaaagcaaagatcacgacaagcagcgaaaccgattcagagatcattaacgcagcggtgcaggacgagaacccgaaacaggccgccgctatagcgaacacgctgatgaagacatttaaaaaagaagtccgtgacaggatgaatataaaaggcgtcattgttttgtccgaggcaaaagcatcggaaagcccgatggtcaagccttcgcgcatcaggaatatcatgatggcgttcggtgcggctctcatggcgggtgtgacgcttgcgttttttctccactttcttgatgaaaccgttaaaagcgagcggcagctcagcgaaaaaacagacttgcctgttttaggggttgtgtatgacatcaaaaatcagcagacacggtctgatgaaaaacatttcggggagtga

SEQ ID NO: 2 - DNA, 753 bp, Bacillus licheniformis
atgaaagaaaatattgattttagagaactgattgcaatcttgcgaaaaagaacggttcttattctcgttttgacaataggtgtaacattgacgaccggaatcattcagttctatgtgctgacacctgtctatcaggcatcgacgcagatcttggtgcatcaagtaggggagaaaaaggggagcgccacatacagcgatattcaaatcaatcttcaatacacacggacattccaagcgcttttgaaaaacccggtgattttggagcaagtcaagagagagcttgatttaccttactctgccggccggttgggtgaaaaaattgcaacgagcagtgaaagcgaatctgagattataaatatttcggtccaagatgaaaatcagaaacgggcggccgatatagcgaacactttaactgcggtgctcaaaaaagagattaagcaaattatgaacaccgatcgggtaaccgtcctgtcaaaagccgaaatagtcgattcgccgacacctgtcagaccgaattacaaaatgaatattttgctggcattcggcgccgcattaatgaccggaatcgctttggcgttctttttggactttatcgatgatacggttgcaagaccgtctcaagtcgaaaaggaagcgggattcatttatttgggaagtattgagcaaatgaagcataaaaaaagtctgtttcgcggggaccccgatatgaatatccgcgtaaaagcaggaaggagtgagccgcttgggtattag

SEQ ID NO: 3 - DNA, 705 bp, Bacillus subtilis
atgaatgagaatatgagtttcaaagaattatatgcgattgtcagacacagattcgtgctgattctgctcatcacaatcggcgtcacccttattatgggttttgtgcaatttaaggtcatttcaccgacctaccaggcgtcgacacaggtgctggttcatgaatcagacggtgaagaaaactcgaatctcagtgacatccagcgaaatcttcagtatagcagcacgttccaatcgattatgaaaagcactgccttgatggaagaagttaaggcggaattgcacctatctgaatcggcttcctcgctgaaaggaaaagtggttaccagcagtgaaaatgaatcagaaattatcaacgttgccgtccaggatcacgacccggcgaaagcggctgagattgcgaacacgttagtgaacaagtttgaaaaagaagtagatgaaagaatgaatgtacaaggcgtacatatattatcagaggcgaaggcttctgaaagcccgatgatcaagccggccaggctgcgaaatatggtcatggcttttggcgctgctgtcatgggcggcattacactggcattttttctgcattttctcgatgatacttgcaaaagcgcacggcagctcagcgagagaaccggattgccatgcttaggctccgttcctgatgtccacaaagggcggaatcgtgggataaaacatttcggggagtga

SEQ ID NO: 4 - DNA, 681 bp, Bacillus amyloliquefaciens
ttgggattcagaaaaaagaagtcaagacggggattggctcaaatatccgttttacaccacaaatcattggtagctgaacaataccgcaccattcggacaaatattgagttttcctctgtacagacccatttgcgctctattctcgtcacttcttccgttccgggagaaggaaaatcattcagcgccgccaaccttgccgcggtatttgcgcagcaggaaaaaaaggtgctgctcgtcgatgcggatttacgaaaaccgacgatacatgagacctatcagcttgaaaatgtacaaggccttacgaatgtgttagtcggaaacgcttccctcggcgaaacggtgcaaaaaacactgatagataatctttatgtcttaacgagcggtccgacgccgccgaatccggctgagcttttgtcctctaaagcgatgggagagctgattcaggagatgtacagccgatacagtctggtcattttcgattcacctccgcttttggcggtggcggacggacaggttttagcgaaccagacggatggaagcgttctcgtcgttttgagcggaaaaacaaaaatggataccgtccaaaaggcgaaagacgcgcttcagcagtcgaaggcgaagcttttgggcgcgcttttgaataaaaagaaaatcaaaaaaacagaacactactcgtattga

SEQ ID NO: 5 - DNA, 702 bp, Bacillus licheniformis
gtgagccgcttgggtattagaaaaaaacgctctcgcaaatatcaatcggcgcttgtcgcattgcatcagccgaacacgccgatcgtcgaacagtatcggacgatcaggacgaacattgagttttcatcatttgagaagccgttcaagtcattgctcattacatcgggcctgccgggagaaggcaaatcattctcggcttcaaacttggcgatcgtattttcgcaacaggaaaaaaaggtccttttgatcgacgcagatttaagaaagccgacgatccataaaatttttgagctcgataaccattcaggtgtcacaaatgtattaatgaaaaaatcgacgctggaaaatgtcgtccagcaaagccaggcggaaaatctccatgtgctgacaagcggtccgattcctccgaatccgtccgagcttttgtcgtcgcaggcgatggaggacctgcttgcggaagcgtacgaccaatacgatttagtcatccttgattcgccgccgcttttgccggtcgcagacgcgcaaatattggcgaatcaggtggatggaagcattcttgtcatcctcagcggaaaaacaaagcttgataacgcgatcaagtctcgggacgcgctgaattcttccaaaagcgaactgctcggcgccgtgctgaacgggcggaaagtgaagaaagcgcgccaatataattacgcaaccatgtaa

SEQ ID NO: 6 - DNA, 684 bp, Bacillus subtilis
gtgttctttagaaaaaagaaagcaaggcgaggtttggctcaaatatccgttttacacaataaatcaattgttgcagaacaatatcgcaccattcggacaaacattgagttttcatccgtccagaccaacttgcggtctatcctcgtcacctcctctgtgcctggtgaaggtaaatcgttcagtgcagcgaatcttgcggctgtctttgcgcagcagcaggaaaagaaagtgctgctggtggatgctgatttaagaaagccgaccatcaatcagacgtttcaggttgagaatgtaaccgggctgacaaatgtgctggtcgggaatgcttcactcagtgagacggtgcaaaagacgccgatcgataacttgtatgtgctgacaagcggaccgaccccgccaaatccggctgaactgctgtcttcaaaagcgatgggagatttaatatctgacatctatgaacaattcagcctcgttatctttgattcccctccgctattggctgttgcggatgcgcagattttagcaaatcagacagatggcagcgtgctcgtagtgttaagcggaaaaacgaaaaccgataccgtcttgaaagcaaaggatgcactggaacaatccaatgcgaagctgttaggcgctcttttaaacaaaaagaaattgaaaaaatcggaacactattcctactaa

SEQ ID NO: 7 - DNA, 1794 bp, Bacillus amyloliquefaciens
atgatttttgcattggatacgtatctcgttttactttccgttgttataggatatcaattttttgaggattcttatcacttttatgactccggagcgctcctgctgaccgcggtgagcatgctgatcagccaccatgtatgcgcttttatgtttcaccagtataagcaggtatggacgtatacgggaataggggagctgcttgccctgctgaaggggatcactctgtccgcagcggtgacggccgccgttcaatatggggtgttccacacgattttgttccggcttttagccgtcagctggatggttcagctattgtttatcggaggcagccggatgatttcgcgcgtgctgaaagaaacgatcggcaagaagcaaaatgactcttcacgggcgctgattatcggcgctggcgcgggggggacgctgctcgcccgtcagcttacgcagaaaaacgatctcggaatcatgcctgtggcttttattgatgatgaccagacgaagcataagctcgagattatggggctgcccgtcatcggcggaaaagaaagcattttgccggcggtgcagaggctgagaattcaccatatcatcatcgccattccgtctctgcgaacccatgagcttcagactttatacaaagaatgtgtgcagacgggcgcccatattaaaatcatgccgcaatttgatgagatcctgctcggaacgcaggctgccggacacatcagagatgtaaacgccgaagatttgctcggcagaaaaccggtcactctggatacgagcaaaatttctgacagcataaagggaaaaacgattctggtcacgggtgccggcggctctatcggttctgagatctgccgccagatcagctcgtttcgtccgcgtgaaatcgtccttctcggccacggggaaaacagcatttattccgtgcatggcgaactgtcagcacgctttgggaaagaggtgctttttcacgcggagatcgccgatattcaggatagagataaaatctttaccttgatgaaaaaatacgagccgcacgtcgtctatcatgcggctgcccataaacatgtgccgttaatggaacataacccggaggaagctgtcaaaaacaacatcctcggcacaaaaaatgtcgccgaggccgccgatatgtgcggaacggaaacgttcgtgctgatttcttctgataaagcggtcaatccggccaatgttatgggggcgacgaagcggtttgcggaaatggtcatcatgaacctcggaaaaatcagccgcaccaaattcgccgccgtccgtttcggaaatgtgctcggaagccggggcagcgtcattccgattttcaaaaagcagattgcaaaaggcggacccgtcaccgtcacgcatccggcgatgacaagatattttatgacgatccccgaagcgtcaagactcgtcattcaggcgggggcgcttgcaaaagggcggcagattttcgttctggatatgggagaacccgtcaaaatcgtcgatctggcaaaaaacctgattcatttatcaggctatacgacagaacagattcctattgaattctccggcatccggccgggagaaaagatgtatgaagaactgctgaatcataatgaagtacatacggaacagatttttccgaaaatccatatcggaaaagcggtagacggggattgggccgtgcttatccgctttatggaagaattcagccgtctgcctgaggaagagctgagaaaacggctgtttgaggcaatcgaatcagtacatgaagaagcggccgcgggcgtgtga

SEQ ID NO: 8 - DNA, 1818 bp, Bacillus licheniformis
ttgacatatcggagaaggctttccattattaccgcactcgattcgtacttggttttgctgtccatctttatcggatatcagctgattttgccatcatatgatttatacccttcggaaatgctgctgatgacttcactgatactgcttggcgctcagcatttattcgcccattgcttccacctttataaaaaagtatgggagtatgcaagcatcggtgaattgtatgtgctgcttaaatcgattacattgtcccatcttgtgacggcggccctcgagctgtttttctttcaaaacgttccggttcgtcttttatgtttaagctggctgttccagctcattttgatcggcggatcgcggatgatgtggcgcatcatcagggaacaggtgaacaaagaaagcaaaggatctctaagggcgcttatcatcggagcgggctctgccggcagtttgattgcaaaacagcttgtgcagaagccggaattgaacattaagcccgtcgcctttatcgacgatgacaaaacgaaataccggcttgaaatcatgggtctgcccgtcttaggcgggaaagagcagattatgcaggcggtccggcaatggaatatcgaccggatcatcatcgccattccatctttgagcgtcactcagatgcaggaaatgtacaaggcgtgtgcgcaaacaggtgtcaaaacgcaaatcatgccgaaaatagacgaaattttgcttggcagacatcctgtcggccagcttcgcgatgtcaaagcagaagatttgcttggaagggagcctgtccagctcgatacgagcgaaatctccaatacggtcaaggaccgcgtcgtacttgtgactggggccggaggttcaatcggctcggaaatctgccggcaaatcagcaaatttaaaccgaaatcgatcattttagtcgggcacggagaaaacagcatccattcgatcctgcttgaactgaaggagaaattcggaaagcatgtcgcctattatcccgaaatcgcggacatacaggacagagaaaaaatgtttttgctgatggaacgctacaaaccgaatgtcatttatcatgcagctgcccacaagcatgtaccgctgatggaaaaatgcccgaaagaagctgtcaaaaacaatatcctcggcacgaaaaacgtcgctgaggccgccgacgaaaccgaagtggagacatttgtcctgatatcgtcagacaaagcggtcaaccctgccaatatcatgggggcaacgaagcggttcgcggaaatgctgatcatgaatctcggcaaaacgagcaaaaccaaatttgtcgccgtccgcttcggaaatgttctcggcagcaggggaagcgtcattccgattttcaaaaaacaaatcgctaaaggcggcccggtcactgtcacacaccaggacatgacgaggtacttcatgacgattccggaagcttcaaggctcgttattcaagcgggggcgcttgccaaaggaagacagatattcgtgctcgatatgggcgaaccggtcaaaatcgtcgacctcgccaaaaatcttatccagctttccggctatacgacagaacagatcaaaatcgaatttacaggcatccggccgggagaaaaaatgtacgaagagctgctgaatcaaaatgaagtgctggcagagcaggtttttccgaagattcatatcggcaaggcggtcgacgtcgaatggacggtgctgaagtcatttatggatgaatttatgtatttgtcagaccgcgagctgagagaacgcttgttcaaagcgatcggccagcacgagaaaaagctggtgacagcgcactag

SEQ ID NO: 9 - DNA, 1797 bp, Bacillus subtilis
atgattattgcgctggatacttacctcgttttaaattcagttattgcaggatatcaatttttaaaagattcctatcaattttatgactccggagcattactgcttaccgctgtcagcttgctcctcagctatcatgtgtgtgctttcctgttcaatcagtataaacaggtgtggacatacaccgggcttggcgagctgattgtcctgcttaagggcattacgctttcagccgctgtgaccggcgtcattcagtatgctgtgtatcacacgatgttcttccgtctgttaaccgcgtgctgggtgcttcagcttttgtctattggagggacccgtattttatccagagtattaaacgaaagcatcaggaaaaaacgctgcgcctcgtcccgcgcgctgattatcggggcgggctcaggtgggactctgatggtcaggcagctgctttcgaaagatgaacctgatatcatacctgtcgcttttattgatgacgaccaaacgaagcataaattagaaattatggggctgcccgtaatcggcggaaaagaaagtatcatgcctgcggtgcaaaagctcaaaattaattatattattattgccattccttcactccgcacccatgagcttcaggtgttatataaagaatgtgtgcgaaccggagtaagcattaaaattatgcctcattttgatgaaatgctgcttggcacacgaactgccggacaaatcagagatgtaaaagctgaggacttgctcggcagaaagccggtaaccctcgacactagcgaaatttcgaaccgcatcaaaggaaaaacagttctcgtcacgggagcgggcggatcaatcggctcggaaatctgccgtcagatcagcgcgtttcagcctaaggaaatcattctgctcggccatggggaaaacagcattcattcgatttatacagagctgaacggacgattcggcaaacacattgtgttccatacggaaatcgctgatgtgcaggaccgcgataaaatgtttaccttgatgaaaaaatacgagccgcatgttgtctatcatgcagctgcccataagcatgtgcctttaatggaacacaatccagaagaggcggtcaaaaacaatattatcggaacaaaaaatgtcgcggaagcagccgatatgtcgggaactgagacattcgtgctgatttcatcggacaaagcggtgaacccagccaacgtaatgggggcgacaaaacgattcgcagagatgattattatgaatcttgggaaagtcagcagaaccaaatttgttgctgttcgcttcggcaatgtactcgggagccgcggcagcgtcattccaattttcaaaaaacagattgaaaaaggcggcccggtgacagtaacacatccggcaatgacccgctatttcatgacgattcccgaggcatcaaggcttgtgattcaggctggggcactggcgaaagggcgtcaaattttcgttctcgatatgggagagcccgtaaagattgtggatcttgccaaaaacctcattcatttgtccggctacacgactgagcaggttccaatcgaattcacaggcattcgtccgggcgaaaaaatgtatgaagaattgctgaacaaaaatgaagtccatgctgaacaaatctttccaaaaattcacatcggtaaagcggtggacggcgattggccggtgctgatgcgctttatcgaggattttcatgagctgccggaagccgacctgagagcgaggctgtttgcggcaatcaatacatcagaagaaatgacggctgccagcgttcattag

SEQ ID NO: 10 - DNA, 1140 bp, Bacillus amyloliquefaciens
atgacgaaaaaagtattattctgcgccactgtcgattatcattttaaagcctttcatcttccgtattttcaatggtttcaggatatgggctgggaggtgcacgtcgcggcaggcggcaatatgaatcttccgttcgttgatgaaaagttttccattccgatccggcgctcgccgtttcatcccgaaaatctttctgtttacaggcggctgaaacggctcattcaggacaacggctacgacatgattcactgtcatacgccggtcggcggcgtgcttgcacgccttgcggcgagacaagcgcgccaaaaagggacaaaggtgctgtacaccgcgcacggctttcacttctgcgacggagcgcccctgaaaaattggcttctttattacccgatcgaaaaatttctctcttcttacacggattgtttgattacgattaatgaagaggattacgaacgggccaagcaaatgaaaaaaacagcttgcggcgcgaagaaaatacacggcatcggcgtgaatacagacaggtttcgccctgtgagccgggcagagagtgaacgtctgagagaaaaacacggcttcggcgccggggaatttatcctcatataccccgctgaattaaacggcaataaaaaccagggacttctcattgaggctgcggcactgctgaagaaccgcattccggagctgaaactcgtatttgccggagaaggcgcgatggaagagccgtacagaaaaaaggctgaatcactcggggtatccgacattgtgcggttttacggtttttgccgtgacattcatgagctgattcagcttgctgatctgtccgtcgcctcaagcatccgcgaagggctcggtatgaatctgcttgaggggatggctgccgaaaagcccgccgttgccgcagacaacagggggcaccgtgagatcatcgaagacggtgtgaacggttttctcgtgccggcaggggacagcgcggcatttgccgatcggattgaaaagctgtaccgatccccgggtctgcgaaaagcaatgggacaagaagggcgcaggacggcggaatgtttttcagaaacacgtacggtaaaggaaatggcgcacatttacgccggttatatggataaaaaggagaaaagcttatga

SEQ ID NO: 11 - DNA, 1149 bp, Bacillus licheniformis
atgacaagaacggttttgttttgcgctactgtggattaccatttcaaggccttccacctcccgtatttaaaatggtttaaagagcaggggtggaatgttcatatcgccgcaaaaggagatatgacactgccctatacagataaaaaatttgatatcgatatcaggcgttctcctttgaatgcaagcaatatcgctgcctatcgggagctggcgcgaatcattgacgaacaccggtacagcatcattcattgccacacgccgatgggaggcgtgctggcaaggcttgcggcccggaaacagagaaaagaggggacgaaagtgatctataccgctcacggtttccacttttgccaaggtgctcctttaaaaaattggctgctgtattatccgattgaaaaagggctgtccgctttgaccgattgcctgattacgatcaatgaagaagatttcgtccttgcaaaaggcttgcgaaaagcgctgcgcacggaaaaaatccacgggatcggcgtcgatacggagcggtttcatcctgtcagtgaaacagagaaaatgctgctcaggaaaacatacggtttcaaagaagacgactttatcctcatatatcccgcggagctgaacgcgaataaaaaccaggccttgctcatcgaaacggcggctgctttaaaagacagagccccgaacttaaaggtcgtgtttgcaggaaaagggcagatggagcaaaaataccgaaatcacgctgaacaaaaaggcgtttcttcgctcgtcatgtttgccggttttcaaaaaaacatccacgaatggattcagcttgcagacgtgtctgtcgcctcaagcatcagggaagggctcggcatgaacctccttgaaggaatggcgtcaggaaagcccgccgttgcagcggacaaccgcggacaccgggaagtcattcaagagggcgtgaacggatttttggttccgcagggagacgccggaacgttcagcgaccggatattgcagctgtaccgtctgccttctttgcgaaaaaagatgggagacgcggggagacgaacagccgccgctttttcccagcagcgcaccgtcaaagaaatggcgggcatttactcttcctttatggataacgaaacagttgaaaggaggctgaaaggatga

SEQ ID NO: 12 - DNA, 1146 bp, Bacillus subtilis
atgacgaaaaagatattgttttgcgcgactgttgattatcattttaaggcctttcacctcccttattttaaatggttcaagcaaatgggctgggaggttcatgtcgccgcgaacggacaaaccaagctgccgtatgtggatgagaaattctccatcccgattcgcaggtcaccttttgaccctcagaacctggccgtttataggcagctgaagaaagtgatcgacacctatgaatacgacattgtccattgccatacaccggtcggcggcgttctcgccagactggcggcgaggcaggcacggcggcacggaacaaaggtgctgtacacagcgcacggatttcacttctgcaaaggggcaccgatgaaaaattggcttctttactatccggttgagaaatggctttcagcatatacagactgcctgattacgattaatgaagaggattacatacgggcgaaaggacttcaaaggccgggcggaaggacgcagaaaattcacggcattggcgtcaataccgagcgtttccggcctgtcagtccgatagagcagcaaagactcagagaaaagcacgggttccgtgaagatgattttatattggtttatccggctgagctcaatctgaacaaaaaccagaagcagttaattgaagccgcagccttgctaaaagaaaaaattccctcactccgccttgtgtttgccggggaaggggcaatggaacatacgtatcaaacgttagctgaaaagcttggtgcctccgcccatgtctgtttttacggcttttgcagcgacatacatgagttgattcagcttgcggatgtatctgtcgcatccagcattagagaaggcctcggtatgaatgtgcttgagggaatggcggcagaacaaccggcgatcgccacagataatcgcgggcatcgggaaatcatccgcgacggagaaaacggttttctgatcaaaatcggtgacagtgctgcttttgcccgccggattgaacagctttaccataagccggagctctgccgaaagctgggacaggaaggccgaaaaacagccttgcgcttctcggaggcgcgaacggtggaagaaatggcagatatttattccgcgtacatggatatggatacaaaggagaaaagcgtatga

SEQ ID NO: 13 - DNA, 843 bp, Bacillus amyloliquefaciens
atgaatgcaggagcacaaccgaaaatttccgtcatcatgggaatttataattgcgaagagactttagcggagagtattgaatccatcctgagccaatcgtataagaattgggaactgattatgtgtgatgacgcgtcaactgacggaacgtatcagattgcgcgccgttacgcggatcattacagcgaccggatcacgctgattcaaaataagacgaatcaacggcttgccgcttcgctgaaccgctgtctgacatacgcaacgggggattatatcgccaggcaggacggtgatgacatctccaacccgaggagactggaaaaacaggccgctttcttaaacaagcacgcccactatcaagtcgtgggcacggggatgctcgtatttgatgaattcggcgtcaggggcgcccggcttttgccgcccgtgccgaaaccgggaattatggcgaaaggcacaccgttctgccacggcaccattatgatgcgggctgaagcgtacaaagcgcttggaggctaccggtcagtcaggcggacgcggcggatggaggatatcgatttgtggctgcgcttttttgaagcgggtttccgagggtacaatctccaggaaacgctgtataaagtaagagaggacagtgatgcgttcaaaaggagatcgtttacgtattccattgataatgcggtgctcgtttttcaggcatgcagacggctgaagctgccgatttcccattacgcgtatatcgcaaaaccgctcatccgcgccattacgccgcccgcggtcatgaaccgttatcataaaaacagggacatccgccaaaaagaagggcttgccgaacatgactga

SEQ ID NO: 14 - DNA, 843 bp, Bacillus licheniformis
atgatagggggtcaaaagccgaaagtttctgtcattatgggagtctacaattgtgagaacacgatcgcagagagcatcgagtcaattttaaatcaaacctataaaaattgggaactgattatatgcgacgatgcttcgacagacgggacatatgctgttgccaggcggtatgccgatcattacgcagataagattaagctgatcaaaaacgagaagaatcagcggctggctgcctcgttaaaccactgtctccaatacgccggcgggaaatatatcgcgcgccaggacggagatgacatctctttgccgaggcggtttgaaaagcaggtcgcgtttttggaatcgcaatctcattatcatgtcgtcggaagcggcatgatggccttcgacgaaaacgggattagaggcgtcagaatgcttccttcctctccagaaccgagaatcatggcgaaagggacgccgttttgccatgcgacgatcatgatgagagccgacgtctacgaggcgctggacggctatcgggtcggccggagaacaagaaggatggaagatgtcgatttgtggcttcgtttttttgaggcgggcttcacgggctacaaccttcaggaagccttatacaaagtaagggaagacgaatcggcgtttaaaaggagaaagctcagctactcgattgataatgcgtttatcgtctttgccgcctgcagacggctgaagctgccgctatcggactatatttatacaatgaaacccatcatcagggggcttatgcctccttttatcatgaacagatatcataaaagaagattgatgaatgaaggcggaggggtcgtaaaacatgaatga

SEQ ID NO: 15 - DNA, 837 bp, Bacillus subtilis
atgaactcaggaccgaaagtttctgtcattatgggcatttataattgcgaacgcactttggcagaaagcatagaatccatactcagccaatcctataaaaattgggagctgattttgtgcgatgatgcgtcaacagacggcacgctccgtatcgcgaagcagtatgccgctcattacagcgaccgcatcaaactgattcaaaacaaaacaaataagcggcttgccgcatcattaaatcattgtctttcgcatgcgacaggcgattatatcgcacgtcaggacggagatgacctttcgtttccgcgccgtctggaaaagcaggtcgcgtttttagaaaagcaccgacactatcaggtggttggcaccggcatgcttgtgtttgatgaatttggcgtaagaggcgcccgcattctgccttctgttccggagccgggcatcatggcaaaagggactccattttgccacggcacgattatgatgagagcgagtgcctaccgcacgctgaaaggctaccggtcggtgcggcggacgagacgaatggaagatattgatttgtggcttcgcttttttgaagagggcttcaggggctataatcttcaggaagccttgtataaagtgagggaagacagcgatgcattcaaacggcggtcatttacgtattcaatcgacaatgccattcttgtctatcaggcgtgcagacgcttgaagcttcctttatctgattacatatatatcgcaaaaccgttaattcgcgcctttatgcctgcagctgtgatgaatcgctaccataaaaaaagagtgatgaaccaaaaggaagggcttgtcaagcatgaatag

SEQ ID NO: 16 - DNA, 1137 bp, Bacillus amyloliquefaciens
atgactgacaaaccgatgcgggtgctgcacatcttcagcgggatgaacaggggcggcgcagaaacgatgatgatgaatttgtaccggaagatggacagaacgaaggtgcagtttgattttttaacccataggaacgatccgtgcgcttatgatgaggaaattctcgctctcggcggacggcttttttatgtgccgagcatcggcagcacaaacccgattacatttgtgaaacaggtcaagcgggtgattcaggaaaaaggcccgtttgccgccgtgcacgcccataccgattttcagtcgggattcatcgcgcttgccgctcgtttagccggcgtcccggtgcggatctgccattcgcacagtacgtcgtggcgggggcgggcgtcgcggcttgccgggatgcagctgtttgttttcagacggctgatcacggcaaacgccacggcactgtgcgcctgcggaaaagacgcgggccgtttcttattcgggaaagaaaaagacgtgcatcttttgccgaacggcattgatctcggcttatttgccggcgggggcgcggacacagaagctgaaaaaaggaagcggggcattgcagacggccgtttggtgatcggccatatcggccgctttacagaagaaaaaaaccatgaatttctgctccggctggcagctgatatgaaagagcggggcatcggtttacagctgattctcgccggagacggtccgcttcgcaccgacatggaaaatctggcggcgaagctggggcttgatgatgatgtgcgttttatcggcatagaagaccgcgttcatgccctgctgaaaacgctggatgtatttgtcatgccgtctctttatgaagggctgcccgtcacgctcgttgaagcgcaggcctccggggtgccgtgcgtgatatcagacggcataacggaagaagccgatgccgggctcgggctggtcaaaaggctcagcctcaaagaaccgcccggacaatgggcatccgcggtgttacgggcagcagaggcggcaaagcctgacggggagcgcattaaagaaaccctcagacggcagggatatgacgcgggagaaaatgcgggggctgttatgaagctttacaatatgaattggaaaaaggaacagtaa

SEQ ID NO: 17 - DNA, 1161 bp, Bacillus licheniformis
atgaatgacggaagcgtgagaccaaaacgggtgcttcacatcgtaagcggaatgaaccgcggcggcgcggagacgatgattatgaatatataccgccacacagacaggcggcatattcaatttgattttatttcccaccgggaagaaacgtgcgattacgacccggagatcatcacgcgcggcggccgggtgttttatgtaccgagcatcggtcggtcgggtcctgtcgcctacatcaaaaacatcagaaggattttggttgagaaagggccttatgccgccgtacacgctcatacggattttcagacgggctttgccgcattggcggccaggctcgccggcgttccggtcagggtctgccattcccacaacacggcctggaagcctaacccccggttttgggatacatggcagcttcttgcattccgccgcttgattttctccagtgctacggctctgtgcgcttgcggcaaagatgccgggcgttttttattcggcgcaaagaagatgggtgaaaacgcggtccatcttttgcaaaacgggattgaacttgaccggttcaaagaagcgaacggcgtttcaaaaacgaatgcgaaaaagagcttcggtatcaaagaagacgcactggtgatcgggcatgtcggccgtttttttgaacagaaaaaccacgcgtttctgctcgggcttgccgcttattgcaagaaatcgggcataccttttcaagcggtgttcgcaggtgacggtccgctgcgcagacagatggaagaaaaagccgctgctctcggtgtaaaagacgacattctgtttctcggcgtcgtcgaagatatcccggctctcatgcaggcatttgatgtatttgtcatgccgtctttgtttgaagggctacccctcgtactggtcgaagcgcaagcgtcagggctcccctgcattgtatcagacaacattacggaagaaaccgatttgggactcggcctgctgcaacgtctcagcttaaatgccggttttgaacggtgggctgaggatatcagccgtgctgctcagccgaaaaagcctgcatggccggaaatagagagaagccttgctgagagaggctatgatgcaaaagcaaatttggcgagactgatggacatctattcaatctccgcagcagaaggacagtga

SEQ ID NO: 18 - DNA, 1155 bp, Bacillus subtilis
atgaatagcagccaaaagcgcgtgctccatgttctcagcggcatgaacaggggcggcgcggaaaccatggtaatgaatttatatcggaagatggacaaaagcaaagtgcaatttgattttttaacgtatcgaaatgatccgtgcgcttatgatgaagagattttatctttaggcgggcggcttttttatgtcccgagcattgggcaaagcaatccccttacatttgtgaggaatgtgagaaacgcgataaaagaaaatgggccgttcagcgccgttcatgcgcacacggatttccaaacgggttttatcgcccttgcggcaaggctcgccggagtgccggtcagggtatgccactcccacaatacgtcttggaagaccggcttcaactggaaggatcgattgcagctgctcgtgttcaggcggctcattttggcaaatgcgacagcgctgtgtgcctgcggagaggatgcgggcaggtttttatttggacagtccaatatggagcgggagcgtgttcaccttcttcctaacgggattgaccttgagttgttcgccccaaatgggcaggcggctgatgaagaaaaagcagcacgcggcattgcagccgaccggctcatcattggccatgtggcccggtttcatgaagtgaaaaaccacgcgttcctgttgaagcttgccgcacatctcaaggaaagaggcattcgctttcagctcgttctggcgggagacgggccgttgtgcggggagatagaggaggaggcgcggcagcagaatttgctatcagacgtcctctttttaggcacggaagaacggatccatgaactgatgcgaacattcgatgtatttgtcatgccgtctctgtacgaaggcttgccggttgtgcttgtggaagcgcaggcgtcggggcttccatgcatcatttcagacagcattacagaaaaagtcgacgccggtctcgggcttgtcacaagattaagtctttctgagccgatcagcgtctgggctgaaaccattgcaagggcggccgccgcaggcaggccgaagcgtgagttcatcaaagaaacactcgctcaacttggctacgatgcacagcaaaatgtaggagcgctgctgaatgtatacaacatcagcacggaaaaggaccataaccgatga

SEQ ID NO: 19 - DNA, 1104 bp, Bacillus amyloliquefaciens
atgattgtatatgtagtcaatatgggaattgtttatatttggtcatggttcgcgaaaatgtgcggcgggcgtgatcaatcgctttcgaccggataccggccgaatcagctcgtcatgatcgttccgctgctatcgctcgtattggtctccggtttgcgctacagagtcgggacggattttcagacgtacgcgctgatgtacaagctggcgggaaattacggaagcatttgggagatcttcgggttcggagcgaaaaaagcagccgttgatccggggtttacggctctcatctggctcatgaattttatcacgaaagatccgcaaatcatgtatttcaccgtggcggtcgtgacgtgcagctttattttaaaggggctcgttgagttcggccgtccgtttgagctgagtgtgtttttgtttttgggcacgtatcactattacgcttcctttaacgggatcaggcagtatatggtggcggccgttttgttttgggccgtccgctacgtgatcagcggaagctggaagcgttatatgtcaatcgtgctgctgtgttcgctttttcattcctcagcgctgattatgattccggtttattttctcgtcagaaaaagagcctggtcgcccgcgattttcggactgtcggcattgtttcttcttatgacgtttctgtatcagaagttcatttcgctgtttgtcgtcgtgctggaaaacagctcatacggccattatgagaaatggctgatgaccaatacgaatgggatgaacgtcgtaaaaatcgcggtgctgcttctgccgctgtttttggctttctgctatcgggagcggctgcggaagctctggcctgacagtgatgtcatcgtgaatttctgtctgctcggcctgctcttcggtcttttggcgacaaaggatgttattttcgcccgatttaatatatatttcggcctgtaccaaatgatcttgatcccttattttgtgagaatttttgatgaaaaatcaaatgcgctgatttatatcgccattattgtctgttattttctttacagttacttgcttatgccggtcgattcgtctgtgcttccgtacagaacgattttttcccggtaa

SEQ ID NO: 20 - DNA, 1104 bp, Bacillus licheniformis
atggctgtctacatgttgaatatggggattgttttcgtctggtcatggtttgcgaaaatgtacggcagggaagatcacaggctgccgacgggctaccgtccgaatgcgatcctcaccgtcgttccgctcgcgtctttgattatcgttgccggcttaagatacaaggtcggaacggattaccacacatatatgctgctttacgaattagccggaaaatacaacagcatttgggagatattcggtttcggaacaggcaagtcgtcgacggacccgggatttaccgcactcttatggattttaaatcagatttcagccgatccggcgctcatgtttgccgtcgttgccgcgattacctatatctatattgtcaagacgctttatgtgtatggaaggccgtttgaattgagcatgtttctgtttatcggcatgtttcactattatgcttcgtttaacggcattcgccaatacatggcggcggccattctgttttgggcggtgcggtatctgatcgatgggaagctggtgcgctatatgatcgttgtactgatctgttcgctttttcattcttcggcattgatcatgattccggtttatttcatcgtcagaagaaaagcgtggtcgcctgtcctctggtgcctgatgctcgtctttttggcggggacttttctgtatcaaaaatttctgtccgtattcctagtcgtgcttgaaaacagtcaatacggacattatgaagaatggctgatgaagaatacaaacggcatgaacgttattaaaatcatcgttcttcttctcccgctcgcacttgccttttgcttcagggagcagctcagaaagcgctggcccgaagtcgattatatcgtaaacctttgtctgatcggtttcctgttcggaattttggcgacaaaggatgtcatttttgcgaggttcaacatttatttcgggctctatcagctgattctcgttccttattttgtacggatttttgaaccgaaatcaaacgcgcttctttatgttttgattttgatctgttacttcttatacagctttatgctcatgccgttcgactcgtcggtattgccgtacagaacgatttttgaacgttga

SEQ ID NO: 21 - DNA, 1104 bp, Bacillus subtilis
atgattgtatatgccgtcaatatggggattgtatttatttggtcttggttcgctaaaatgtgcggcggccgtgatgattcgcttgccacggggtatcggccgaataagcttttgatctggattccgctcgcttcacttgtgctcgtgtcaggtctccgctatcgagtcggcacggattttcagacgtacacgctgttgtacgaattggcgggcgattatcaaaatgtgtggcagatattcggtttcggcacagcgaaaacagcgacagatccggggtttaccgcactcctttggctgatgaatttcatcacggaagatcctcaaatcatgtattttacggtggcggtcgtgacctacagctttattatgaagacactcgccgactatggcaggccgtttgagctgagtgtctttttatttttgggaacctttcattattacgcatcttttaacggcatcaggcaatacatggtggcagctgttttgttttgggcgatccgttatatcattagcgggaactggaagcgatatttcctgattgtgctggtcagctcgctctttcattcgtcggcgctgattatgattccagtgtactttattgtcagaagaaaagcctggtcaccggcgatattcggcctatccgctttatttctcggcatgacatttttatatcaaaaatttatttctgtgtttgtcgttgtacttgaaaacagctcatacagccattatgaaaaatggctcatgacgaacacaaatggaatgaatgtgatcaaaatcgctgttttggttctgccgctgttccttgcattttgctataaagaacgactgcggagtctgtggccgcaaattgatattgtcgtcaatttgtgcctgctaggttttttgttcggccttttggccacaaaggacgtgatttttgccagattcaatatttatttcggtctgtatcaaatgatcctagtcccttatttcgtcaggatatttgatgaaaaatcgaacgctcttatctatatcgctatcgttgtttgttattttctttacagttatttgcttatgccggtcgattcatcggttctgccttacagaacgattttttcccggtaa

SEQ ID NO: 22 - DNA, 1038 bp, Bacillus amyloliquefaciens
atggaaacacccgcggtcagtttattaattgctgtgtataatacggaaacttatttagaaagatgcttagattcattgctcaatcagtcgctcgccaatatcgaaatcgtggccgttaatgacgggtcaacggatcaaagcccggcggttcttgaagcctatcagaagcgggatgagagaatccgggtcattcatcagcaaaaccgtgggctcggcgcggtccgcaacaaggggatcgaggcggcgcgcggcgaatttatcgcttttattgatgcggacgactgggttgaacctgattattgcctgcgcatgtatgaaaaagcgaaggctgatcaggctgatctcgtgatttgcgaatatgcggccgagtttgcggatacggggaaaaccgccgtttcaacgatcgcttcagcgtacgcgggccgtccgaaacaactgtacctgaaagatttatttgaaggaagagtcagcggtttttcatggaacaagctgtacaaaagaaccatgatcgagcatcatcagctgcgctttccgcttcgtgatgagctggaacacgtggaggatcagtacttcagcctccgggcgcatgtttacgcaggtgcggtctcgtacgtggatcagccgctctatcattatcggattcacttgacttccattgtgcaaagctatcagaaaaagctgtttgactcaggtctcgttctctatcggctgaacgagacttttttgcgtgaaaacggctgcttacaggaataccggcaggagctggattttttcatcgttcagcacggaacagtctgcctgctgaatgaatggaagcggaataatggcggccggttttctgagaagtggaagaatatcagccggatatgcgctgatcccgtgtttcgcctgagcctttccaaaaccggaacggcgccgtttgacgccaaacgctcatgccttctgcttttggcgaagctgaagctgattccgttcgtgtcgctggcatcagccgcgtatcagcgggcaatcgagtacaaaatgaaaatcagagggtga

SEQ ID NO: 23 - DNA, 1032 bp, Bacillus licheniformis
ttggaaaatccagcagttagtctactggtcgctgtttataatacagaagcttttttgccgaattgtttgcagtcgctgatcagccagaccttgaaaaatattgaaatcattatcgtcaatgacggttcaacagacggaagccagaagatcattgatcattacgcccgaaaggacgggcgcattaaaacgattcagcaggacaatcagggcctcggcgctgtccgcaataaaggcatcgaagcggcgtcaggcgagtacttggcattcatcgactccgacgactggattgagccggactattgccagtctatgtatgaaaaagcgaaggatgaggatgccgatcttgtcatctgtgattatgccgttgaaattcaggataccgaaaaaacggtctgcccggacatcgggaagaactatgaaggaaagccgaaagaggcatttatgaaggatttgttaaagggtaaagtaagcggcttttcctggaacaagctctacaggcggagtctgattgagcggcataagctcgttttcccgctccgcgacgagctggaaaacatcgaagatcaatatttcagcttcagatgccttctatatgcgaataccgcggcgttcgtgactaagccgctttatcattacagggtccacctggcgtccatcgtccaaaagtatcaggcagggctgtttgaagacggtctcgctctttatgaagcaaacctggactgcctgacaaagcacggagagctcccggctttaaaggaggcgctgcacgtctttatcgtcaaccacggctgcatcagtattttaaatgaatgcaagagccgaaacaaaaacccttcaatagaaaaatataaaaatatccgcagcatatgcgcctgtccggaattcagggggaaaatctccgcagtggacatgtcggcattcgattccaagaagaagcttttgcttatgctgattcgcttgcggttgatgccggcagtgtatggatttgcggccatttaccagaaaatgatcgagcacagaatgaagaaatag

SEQ ID NO: 24 - DNA, 1035 bp, Bacillus subtilis
atggaaacacctgcggttagtctgttagtcgctgtttataacacagaaacatatatcagaacgtgtctcgaatcactgcggaaccagacaatggacaatattgaaatcatcattgtcaatgacggttcagctgacgccagcccggatatcgcagaagaatacgccaaaatggacaacaggttcaaggtgattcatcaggaaaaccagggactcggtgcggttcggaataaaggcattgaagctgcacgcggcgaatttatcgcgtttatcgattcagacgattggattgagcctgattattgcgagcagatgctccggacagcaggcgatgaaactgatctggtcatttgcaattacgccgcagagtttgaggacactggcaaaaccatggactctgacattgcccaaacctatcaggatcagccgaaggagcactatatcaaggcgttattcgaagggaaggtcagagggttttcatggaacaaactgtacagaagaagcatgattgaagcccatcggctgtcgtttccgctccgaggcgagctggagcatgtcgaggatcagtttttcagcttcagggctcattttttcgcccgctcagtatcctacgtaaaaacgccgctctatcattatcgaattcacctttcctccattgtgcagcgctatcagaaaaaattgtttgaatcaggacttgcgctgtatgagacgaatgcggcgtttttacaggagaacaacaaactggaggagtatcgcaaggagcttgatacctttatcgttcttcacagcagcatctgtatgctgaatgaatggaaaacgagcggcagccgccggctgtttgaaaagcttagaaatgttggcgtgatatgcgcggacccggtgtttcaagaaagtctttcaaaaacgggtactgctccttttgacgcaaaacggtcatgcctgcttctgatggcgaaatacagaatgattccgttcgtcgctatggcatcggctgtgtatcagcgggtgatcgagtacaaaatgagaaacagagggtga

SEQ ID NO: 25 - DNA, 1077 bp, Bacillus amyloliquefaciens
atgtcgatacaatctttaaaaatcaatctcgcggaatggctgttgcttaaggtcaaatacccgtcccaatttcatttcggaacggcagcggatggggcggagcttacagcggcaagaaaaaagatcattctgacgctgctcccgtcccacgataatctcggcgatcacgccatcgcctacgcaagcaaaacgtttcttgaacgggagtatcccgattttgacattgtcgaggtggatatgaaggatatatacagatccgcaaaagcactgatcaaaaagcgccaccccgatgatatggtattcatcatcggcggcgggaacatgggggatttgtaccggtatgaggaatggacacgccgctttgtcataaaaacattccggcaatatcggatcgttcagcttccggcgacggctcacttttctgaatcaaaaaagggacggaaggaactgaagcgcgcccgaaaagtgtacaatgcgcatcccgaccttttgttaatggcgagagatgagacgacgtatcaatggatgaagcgccattttcccggaaaaacagtgctcaaacagcctgacatggtcttatatttggataaaagcgagcggggcataccgcgggaagggatctacctctgtttacgggaggataaggagagtgcgctgacggctgaggacagaacgatggtaaaagaagctttggcaaaagagtacggagagctgtattcctttacgacgacggtcggccggagggtcagccgccatacgcgtgaaaaggagctggaggcgctttggaatacgctgaaaggcgcggaagccgtcgtcactgacaggcttcacgggatgattttttgtgcgctgacgaaaacgccttgtgtcgtcattcgttcgtttgatcataaggtgatggaaggctatcaatggctgaaaaacataccgaacatgacattgcttgaacgtccagaccctgaagctgtaacggccgccgtaaaccggctgctcagcggcaagcatgaggaaggcggttctttgcgcagtgtttattttgccgggctgcgctcgaaaatcagcggtgatgcccaatga

SEQ ID NO: 26 - DNA, 1080 bp, Bacillus licheniformis
atgacctttcaggaactgaagatcaatcttgcagaatggcttttgctcaaggtgaaatatccttctgaatatgtgatgggaacgccgggtttaaggcgctttgaacaatataaaggcaagaaaaaaatcattctgactttaattccgtcccatgataatttgggagatcatgcgattgccctggcaagccgcacattcatagaaaacgagtttcccgattttgagttaattgaaatcggcattaacgatatttacaaacatgcaaaagcgctcatgcgcatccgccaccccgaggatatggtattcatcatcggcggaggaaacatgggggatttgtaccgcaatgaggaatggacgaggcgcttcattatcaaaacattcaagcattataaaatcgtccagctcccggcaactgcgcacttttccgagacgctccgcggcaaaaaagagctgaaaagggcgaaaaaaatctacaactcccatcgccggctgttcatgatggcgcgcgatgatacaacctatcaatttatgaaacagcacttctcaaaccaaacgattgtaaagcagcctgatatggtgctttatttaaagaaagaacagcagtctgaaagagaaggtgtgttggtttgtttaagggaggataaagaaagctttctcaggccggaagagcgtaaaaagctgctgaaggctgtcggcgacgagtatggcggtgcaaaaacttttacaacgacgatcggcaggagggtcagccgcgtgtcccgcgagaaggagctgaaccggctttgggatcagctgagaggcgcggaggtcgtcgtgacagacaggctgcacggcatgattttttgtgcgataacaggaacgccgtgcgtcgtgatccgctcttttgatcataaggtgcttgaaggctttcgctggctgaaagacgttccttcgatgaagcttgtggaaaatcccgatgcggcagaggttctcggcgcaatcgaagagctggtcaagaccggtgactcgcatcgtgagaccccggcaagggatcattatttcgcagatttaagacggaaaattatgggtgatgtccaatga

SEQ ID NO: 27 - DNA, 1077 bp, Bacillus subtilis
atgtcgttacaatcgttgaaaatcaattttgcagaatggctgctgctaaaggtcaaatacccgtcccaatattggctgggagcggcagatcaaccggtaaaggccgcagcacatcagaaaaaaatcatactgaccctgctgccgtcccatgacaatttgggagatcacgcaattgcttatgccagcaaggcatttcttgagcaagaatacccggactttgacatcgtcgaggtcgatatgaaggacatttacaaatcagcaaaaagcctgatccgctcgcgccatccggaggatatggtctttatcatcggcggcggaaacatgggggatttataccgttatgaggagtggacgcgccgcttcatcattaaaacattccatgactatcgggttgtccagcttccggcaacggctcatttttctgacacgaaaaaagggcgcaaagagctgaaacgggcacagaaaatttataatgcgcaccccggcctattgctgatggcgcgggatgaaacaacgtatcaatttatgaaacagcattttcaagaaaaaacaattttgaagcagccggacatggtgctgtatttagacagaagcaaggctcccgcagaacgcgaaggggtttatatgtgtttgcgcgaggatcaggaaagcgtgcttcaggaggagcagaggaaccgggtcaaggctgcgctatgtgaggaattcggcgagatcaaatcctttacgacaacgatcggccgccgggtcagccgcgatacacgcgaacatgaacttgaagcactgtggtctaagctgcaaagcgcagaagccgtcgtcactgacaggcttcatggcatgattttttgcgcgctgacaggaacgccgtgtgttgtcattcgctcctttgaccataaggtgatggagggctatcaatggcttaaagacatcccgttcatgaagctgatagaacatccggagccagagcgcgtaacagccgcagtcaatgagcttttaacaaaagaaacatcccgtgcaggctttccgagagatgtgtattttaaaggtctgcgtgacaaaatcagcggtgaagcgcaatga

SEQ ID NO: 28 - DNA, 1035 bp, Bacillus amyloliquefaciens
atgaacgcgccgctggtaagcgtaatcgtcccgatgtataagacagagccttttattaaagcatgcgcggtatctttaacaaaacagatgctgagagacatcgaaattattttcgtcaatgacggctcaccggatcagtccggccggatggccgaacaattcgccgcagaagatgcgagaatccgggtgattcatcaagaaaacggcggtctcagctcggcccgcaatgccggaattaaagccgcccgcggccgttatatcggctttgtggacggcgatgactatgtgacggaaacgatgttcgagcggctttatgaagaagcggagaagaaccggctggatatcgccggatgcggctattataaggaaacgccgtctaaggaaagagcgtatatgccgccgtccattccgccgggccgcgtcttcacagcggctgagatgactgatctgctgacctgcgcgcacgaacatcggtttatttggtacgtatggcgttatatataccggagagaagtattgcaagggctgctgtttcatgaagatatccgatttgccgaggactctccgtttaacttggcagcgttccgccatgcagcacgtgtcaaagtgatagatgaagggctttatatttaccgggaaaaccccacaagccttacagaaactccgtttaagccacacttagatgacgaactgcaaaagcaatatgaagcgaaaatggctttttatgaggcgaacgggctgacggacgcctgccaaagcgatatcaatacgtatttgtgtaagcatcagatcccgatgctgatcgccaatgcatgcgccgccccgcagccgtcacatgagattacggcgcacatcggcagaattttaacgtatgacatggtaaaaacggcggtgcgcagcacgccgtgccggcataaacagctgctggccggggaacgtgtggtgctcggtctgtgcaaaatgcgccttccgctgcttctgcatgcatttttcgatcggaaaacgaaagagaaaggcagtgcggaaggagcatga

SEQ ID NO: 29 - DNA, 1020 bp, Bacillus licheniformis
atgaagccatttatcagtatcattgttccgatgtacaatgtcgaagattatatagaagaatgcgtcgattcgctgcgcaggcagacgctgaaaaatattgaaatcatccttgttgatgacggctctcccgacagatccggggagatagccagaacgtactgcagcctggatgccagagtgaaagtgattcataaaaaaaacggcgggctgagctcggcgagaaatgccggtcttcaagccgcgacaggagattatgtcgggtttgtcgacggtgatgattttgtattgcctgccatgtttgaaaacatgtacgccgctgccaaaaaagacgacctcgatatcgtcatgtgcgggtatcataaacattccgatacggaagacgcctacttcccgccgccgctgccgaccgatcgcctcttgttgagctgggatattaaacgcgagctcaaaaaggcgcacgaaacccgcttcatctggtatgtgtggcggaatttgtacaggcgcgacctgctgaagaaaaaccagctgtacttttttgaagacattcgttttgcagaggattcgccgtttaatttgtacgccttttatgcagcgaaacgagtgagagcgatcgatgaaggctactacatgtacaggtgcaacccggacagcctgacagaggcgccgtttaaaccgtacatggatgaaagcctgaaaaggcagtaccgggcgaaaaggagattctatgagacgtttcagcttttggatgaatgtgcggacgatttggaaacgtacacatgcaaacatcaaattccgatgcttttagccaatgcctgtgcggaaccgaagccttcaaagcaggtgaggcggcacattaaagacattttgtcttaccggatggtgcagtcgtgcgtcaaagccacgtctctccgcaaccgcaacctgttaatcggccagcggctcgttttattgctatgcaagctgaatattccgattctgctcgaattgttttttaaacgaaatctgcccagtaagggatga

SEQ ID NO: 30 - DNA, 1035 bp, Bacillus subtilis
atgatcccgctcgtcagcattattgtcccgatgtataatgttgaaccatttatagaagagtgcattgattctttgcttcgtcaaacgctttctgatattgaaatcatcctcgtgaatgacggaacaccggatcgttcaggcgaaattgcagaggactatgcaaaacgggatgcgagaatccgggtcattcatcaggcaaacggcgggcttagttcagcgcgaaatacgggaataaaggccgcgcggggcacttacatcggctttgtagacggagacgattatgtatcatccgccatgttccagaggctgactgaagaagcggagcaaaatcagcttgacatcgtcggatgcggtttttacaagcagtcatcggacaggcggacatatgtgccgccgcagcttgaggcaaaccgcgtgctgacgaaaccagaaatgactgaacagcttaaacatgctcacgaaacgagatttatctggtatgtatggcgttatctttaccgtcgtgagctttttgaaagggcgaatctgctgtttgatgaagacatccgttttgctgaagactctccctttaatttgtccgcttttcgcgaagcggagcgggtgaaaatgcttgatgaaggattgtacatttatcgtgaaaacccgaacagcctgacagaaatcccttataagccggcgatggatgaacatcttcaaaaacaatatcaggctaaaatcgcattctacaatcactacggcttagcaggcgcatgtaaagaagatttgaatgtgtacatttgcaggcaccagcttccgatgcttttggcaaatgcctgtgcttctccgaattcgccgaaagacatcaaaaagaagatcagacagattttatcctatgacatggtgcggcaggctgtcagacatacaccgtttcagcatgagaaattattaagaggagagcgtttggtattagcactgtgtaaatggcggctcacttttctcatcaagctgtttttcgagcagcgggggacaatgaaaggcagtgcgaagcaggcatga

SEQ ID NO: 31 - DNA, 1518 bp, Bacillus amyloliquefaciens
atgaaatttgccattaatttcggcgccaatgtgacggcgtttttactatcggtgtttttatcagtgtggatgacgccctttattgtgaaaacattaggtgttgaggcttttgggtttgtccatctgacacagaatatcattaactatttctctattattaccgttgcgctgagctcagtcgttgtgaggtttttctccgtcgccgcccacagggggaacagggacgaggccaatgcgtatgtaagcaattatttagccgcgtctgtcgtgatatctctattactcgctgtgccgctcgccgggactgctttttttattgacaggattatgaatgtaccggccgggctcttaacggatgtgaggctgtctatcgtaatcggcagcgtgctgtttatgctgacgttttttatggcggggttcgcgacggggccgttttttgccaacaagctttatatcacaagttccattcaggccgtgcaaatgctggtcagggtgctgtgtgtgctcgctctgtttacgtgcctgccgccgaagatctggcagatccagctttcggcattagcgggagccgtctgtgcagctgtgctgacttttttcttctttaagaaattgattccgtggttttctttcagccgtaaaactttatcattacagacaagcaaagtgctgttttcggccggtgcctggagttctgtcaaccagatcggcgtgctgctgtttctgcagattgacctcatgacggccaaccttgtgctggggccgtctgaagcgggtgtctatgcggcgattattcaattcccgcttctgctcagaagtcttgccgggacgcttgcttcactgtttgcgccggttttgacttcctattattcaaaaggcgatatggaaggtctcctgtcttacgccaataaggccgtgcggatgaacgggcttctcctcgcgcttcccgccgcgttattaggagggcttgccgaaccgtttctggcgatttggctgggcccgtcgtttgtgcagacggcgccgcttttatatattcacgccgcgtatttggcggtcagtctctctgtcatgccgcttttttacgtatggacggcttttaataaacaaaaaacgcccgccgtcgttactttatgtttaggcggactgaatgtcatattggccgtggttctcagcggaccggcacatctcgggctgtacggcatcaccattgccggggctgtctccctcattttgaaaaatgccgtcttcacgccgctgtatgtttcgcatattaccggctttcagaaaaccgcgttttacaaaggcatgttcggcccgttggcggcggccgtatttgcctgggccgtctgccggggtatccggctgttttcaccgcttgacggctgggccggtctgatcgccgcggggcttgccgtatgcataagctatgccgcattcgcttttttcttcatttgtacgaaagaagaaaggcggcttgctttacagaaatgccgaaaagtgaaaggagctgttcagatttga

SEQ ID NO: 32 - DNA, 1548 bp, Bacillus licheniformis
ttgaataagacatttgttttgaatctcggcgccaatatggcgtcttttttattatcggtgctgttttcgatgtggctgacgccttacgtcatcaagaccctcggtgttgaggcgtttggatttgtccatcttacacagaatatgatcaattacttttcgataattacggtcgccctgagcgcggtcgttgtccggtttttttctgtttccgcccacaggggagcgcttgatgaagcgagaggctatatgaatacctatatcgtttcatcactggtgctttcagtcatcttgttttttccgctcggcggcacggtgttttttatcgatcagatcattcgcgttcccgccggtcttttgggggatgtgcaggtcgcgcttttgatcggcagcctgctgtttttattgacctttgtgatgtcggggtttgccgccggtccgtttttcgccaataaaatctatattacgagcacgatccaagccatccaaatgctgattcgcgttctgagcgtattgttgattttcgcgtggtttgcaccgaagatctggcacattcagctcgccgctctcatcgcaaccgcttcggcgtgcatcctgtcgatctttttctttaagagattgattccatggtttacgttccgggtgagggatatgtcgtttgcaaagtgcaaaaaactgcttcaggcgggaggatggagctccgtcagccaagtcggcatcctgctgtttctgcaaatcgatttaatggtggcgaatgtgatgctgggggtttccgaatccggcatgtacgcggcgatcattcagtttccgctgttgttgcggacgctttcgggaacgctagcggccgtattttcacctacgattacactctattattcaaaaggcgacaaagaagggctcgtccgttatgccaatcaggccgtcaggtttaacggcatcctgctcgctttgccggcggcgcttctgggggggcttgccggcccgtttctgtcgctttggctcggcccttcattcgaacatttgaaatggctcttactgatacatgcgggctatttggttgtttcattaagcccggcgccgctgttttacatctttaccgcctataacaagctgagaacaccggcgctgaccacggttgccttcggcgttgtcaatttgctgcttgccatcgtgctgagcggcccggcgggactcggcctttacggaattgcgctggccggtgcggcagctttaacgctgaaaaacgtcgtcttcactccgatttatgcatcaaagattacgggtgaaagaaaaagggtcttttataaaggcatatacgggccggttgccggcgcttcatttactttggctgtctgttacgctcttcaatacttattttcgatcgtcagcctgctgtctttgtttgtcacggcactggctgcgacgttggcttacggcctgtttgcctatttcgtcatgttgacgaaagcggaacggcgcattgtcacaacgaagctgcaagcataccggtgttctctatccttcccgtttcaaaagggattttttaaataa

SEQ ID NO: 33 - DNA, 1518 bp, Bacillus subtilis
atgaaattcacgataaatttcagcgcgaatctcacggcttttctcttgtctgttttcctatcggtttggatgacgccttttattgtcaaaacgctcggtgtcgaagcgtttggctttgttcacttgacacaaaatgtgattaattacttttcggttattaccgtggcgctcagctcggttgtcgttcggttcttttctgttgctgcccacaggggagagcgggagaaagcaaatgcgtatatcagcaattatttagccgcctctgttttgatttccttgctgctcttgctgccgcttgcgggttcggctttttttattgaccgcgtcatgaatgtgccgcaagcgcttttggcagatgtgcgtttgtcgattttaatcggcagtgtgctgtttattttaacgtttctgatggcgggcttcggcgctgcaccattttatgccaaccgcctttatatcaccagttccattcaggcggtgcaaatgcttatacgggtgctgtctgtgctgctcctgtttgcatgctttgcgccgaaaatctggcagatccagcttgcagctttagctggtgctgttattgcgtctgtgctgtctttctatttcttcaaaaagctgattccgtggttttcgtttcgtatgaaggatctttcattccgtacaagcaaggagctgtttcaagcgggcgcatggagctccgtcaatcaaatcggcgtcctgctttttttgcagattgatctgttaaccgccaatttgatgctgggggcgtctgcatccgggaaatacgcggcgattatccagtttccgctgcttttgcgcagcttggccggaacggtcgcatccctgtttgcgcccatcatgacttcatattattcaaaaggcgatatggaaggattgatgaattacgccaataaggcagtaaggctcaatggtcttttgcttgcacttcctgctgccttattgggcggattggcgggaccttttctgacaatctggctcggaccgtccttttcaacgatagcaccgcttttatttattcatgccggatacttggttgtcagcctcgcctttatgccgctgttttatatatggaccgcttttaatcaacaaaaaacaccggcgattgttaccctgctgttaggtgcggtgaatgtggtgctggcggtcacgctgagcgggccggctcatctcggtctgtacggcataacattggcaggggccatttctcttattttaaaaaatgccatctttacgccgctttacgtatcccgcattacaggctacaaaaagcacgtgttccttaaaggcataatcgggcctctttcagccgctgtatttgcctggacggtctgtaaggcaattcagttcattgtgaagattgacagctggccgtcattgatagcgacgggagtgacagtcagcttttgctacgctgttttcgcttttatgctcgtttgtacaaaagaggaaagacagctggtattaaaacggtttcgaaaaacgaaaggagctgtgaatctttga

SEQ ID NO: 34 - DNA, 609 bp, Bacillus amyloliquefaciens
ttgaaggtgaaacgtgtatttgatatagcggccgcgacgctgttattgtgcggcgcgagtgtcattcttctcttcgccatggcagccgtcagatgcgccatcgggtctccggtattgttcaaacagacgagaccggggcataacgggcgtccgtttacgttgtataaactcagaacgatgacggatgcgcgtgatgaaaacggtgtgctgcttccggatcatctgcgcctgaccaaaacgggaaggctgatcagaaaactgagcatcgacgagcttccccagctttttaatgtgctcaaaggcgatatcagtctggtcggcccccgccctttgctgatggattatctgccgctttacacggcggagcaggcgcggcggcacgaggtgaagccggggattacgggctgggcgcaggtcaatgggagaaacgcgatttcatgggaggagaaattcaagctcgacgtgtggtacgtcgacaaccgcacctttcttctcgacttaaagattctgcttttaacagtgaaaaaagttctcgtgtcagaaggcatacaccaagccggccatgtgacggcaaagcgcttcacaggcagcggagatatgtcatcgtga

SEQ ID NO: 35 - DNA, 603 bp, Bacillus licheniformis
gtgctagcgaaacggtttttcgatcttgcattgtcagttattttgctggtcgcgctcagtccggccatgattctgacggcttgtctcatcagatggaaaatcggctcgcctgttttatttcgccaaacccgtccggggctgaatggcgagccttttacattatataaattcagaacgatgactgatgaaagagatgcagcaggcaatctgctaagcgatgaaaagcggctgacaaagacggggcggctgatcagaaaaacgagccttgacgaattgccgcagctgatcaacgttatcaaaggagacctcagcctcgtcgggccgcgcccgcttttgatggagtatatccccctttatacgaagcggcagtggagacgccacgaagtcaagccgggcatcacgggctgggcgcagatcaacgggagaaataaagtgacatgggaggaaaaatttgaactcgatgtctggtatgtggatcatcgttcttttttgcttgatctcaaaatccttttgttgactgtcgtaaaggttttgaagtcggaaggcgtcagccaggaccggcatgtgacagcagaaaaatttacagggagaaggaatgcctga

SEQ ID NO: 36 - DNA, 609 bp, Bacillus subtilis
ttgatcctgaaacgactttttgatctgacggccgccatttttttgttgtgctgtacaagtgtgatcattctgttcaccatcgccgttgtcaggctgaaaataggatcacctgtcttctttaagcaagtaaggccgggcctgcacggcaaaccgttcaccctatataagttccggacaatgacggatgaacgggacagtaaaggaaatctgctgcctgatgaagtccggctgacgaaaacgggcaggctgatcagaaagctgagcattgatgagcttccgcagctcctgaatgtcctgaagggcgatctgagccttgtcgggccgcggccgcttttgatggactatctgcctctttatacagaaaaacaggcacggcgccatgaggtgaagccgggtatcacaggctgggcgcaaatcaatggcagaaacgcgatttcctgggaaaagaaatttgaattagatgtttggtacgttgacaactggtcattttttctcgatttgaaaattttatgtttgacggtgcgaaaggtccttgtttcagaagggattcagcaaaccaatcatgtgaccgcggaacggtttaccggaagcggagatgtgtcctcatga

SEQ ID NO: 37 - DNA, 648 bp, Bacillus amyloliquefaciens
gtgaaaaaggtcgtgctgatcggaaacggcggacacggaaaagttgtaaaggaaatcgtgaaggctcgttccgatatggagcttgcgggcatattggacgacggattcagcggttttaccgttcgggacggtttgtatacgggccgaacgaaagacgtgcacatgcttcggaaactcgtcccgggagccgtattcacgatatgcatcggtcacaatggtgtaagaaagcagctcgctgaaacgcttgggcttgaacatgatgattacaccgctctcattcaccccggggcaatcgtcagtgatacggcttccgtcggacacggaacggttgtgatggcgggcgccgtcattcaggcgggcgctgacatcggcgcgcattgcatcatcaatacaggtgcggttgccgatcatgacaatgcaatcggagattacgtccatctttccccgcgcgctgcgcttgccggcggagtgaaagtcggggaaggagcgcacatcggaatcggcgcgtctgtcataccgcggacagacatcggtccttggtcggttatcggcgcgggggctgccgtcatcagccggattcccgatcatgtgacggcggtcggcgttccggcccgcgtcatctcctctattcataatgaaaaaggatga

SEQ ID NO: 38 - DNA, 627 bp, Bacillus licheniformis
atgcaaaacgtggtgatcatcggagccggcggccatgggaaagtcgttcgggaacttgtaaaagagcggccggatacggagcttgccggtattcttgatgaccggtatgcggagcttcatgttgagaacggtctgtatcgagggccttcagctgccgcagaagaacttgcgcggcttcatccggacgcgaagttcgtcctggctgtcggccaaaacagcatcagacagcagctgtatgaacgcattggtcttccgcttgaccggtatgcggttttgattcatccctctgctgttgtcagcggttcggcccggattcaaaacggcgccgttgttatggcatcgagcgtcatccaagcggatgcagacgtcggcatccacgcgattgtcaacacaggtgcgatcgtcgaacacgacaatcggatcggcgattacgttcatctttcgcccggaacggtgttaaccggcggcgtgacagttatggaaggcgctcatctcggcgcgggaacggcggtcattcccggaaagacagtcggacgctggagcgtgacgggagcgggggcagccgtgattcacgacattcctgataattgcaccgcagtcggagtccctgcaagaatgatcaaataa

SEQ ID NO: 39 - DNA, 651 bp, Bacillus subtilis
atgaaaaatgtggccattgtgggtgacggcggtcacggaaaggtgatcagagagctgataaacgcccgctcagatacgcgcttagccgcggtgctggatgataaattcaaaacgttcgaaggcggaaaagaatggtacacaggaccgccgaaagccgttactgaactgcgcaggctcattcctgatgtgctgtttctgattgctgttgggaataacagtgtcagaaaacagctggcggagcgactgggactggggaaagatgattttattacattgattcacccgtcagccatcgtcagcaagtcggctgtcattggggaagggacagtgattatggcgggcgcgatcattcaggcggatgcgcgcatcggcgcccactgcatcatcaatacgggtgcagtggcagagcacgacaatcaaatcagcgattacgttcatctgtccccgcgtgccacgctgtcaggagcggtttccgttcaggaaggcgctcacgtcggaaccggtgcatccgtcataccgcagatcataatcggggcttggagcattgtcggagccggctccgcggtgatccgttccataccggacagggtaacggcggccggtgctccggcacgcatcatttcttccattcaaacatcaaacaaaggatga

SEQ ID NO: 40 - DNA, 1173 bp, Bacillus amyloliquefaciens
atgcaaacaaacaaacggatttatttgtctccgcctcatatgagcggaaaagaacaggaatacatcgcagaagccttccgttccaattggattgccccgctcggtccgctcgtcaattcgtttgaagcgcggctcgcggaatatgcgggagtgaaaagcgcggcggccgtaagctcgggaacggccgccatccatcttgccctgcggcttgcgggtgtgaaaaaaggagacgttgtattttgtccttcctttacgtttgtcgccacggcgaatccgattgtgtatgagcaggctgagcccgtttttattgattcggaatgggagacgtggaatatgtcgcccgatgcgctggagcgggcgctgcgggatgcaaagcggcgcggcagactcccgaaagcggtgatcgccgtcaatctctacggccaaagcgcgaaaatggatgaattaatgagcctttgtgacgcatacggcgtctgtctgattgaagacgctgccgagtccttaggatcaacatataaaggcaggcagagcggcacgttcggccgtttcggcatttattcctttaacggcaacaaaatcattacaacatcgggaggcggcatgctcgtgtctgacgatgaagccgcgattgaaaaagcgaggtttcttgcgtcccaggcgagagatgccgccgttcattaccagcacagcgagctcgggtataactacaggctcagcaacattctcgcgggcgtggggatatcgcagctggaagtgctcgaagatcgcgttcgggcgaggagagaaatctttcacaggtatcgggaagcgctggaaacatatccgggcatccgcatgatgccggagcttgaaggaacggtttcaaaccgctggctgacggctctgacgcttgataacggcgtgacgccggaagaagccgtcgcctgtcttgcggaacagaacattgaggcccggccgctgtggaaaccgctgcatacgcagccgcttttctcatcttccgtcttttatcctcacagcgaacatgagcgggtctctgagaaccttttcagtcggggtatctgtctgccgtccgggtctgacttgtcttctgaggagcagcagcgtgtcattgacgcacttgcgcaactgtttgaaacgaaaggggagaaaacatggacagccgccatgttatga

SEQ ID NO: 41 - DNA, 1146 bp, Bacillus licheniformis
atgagtcagaataagcgaatttatttatcaccgccgcacatgagcggagacgaggagcgctatgtagccgaagcgtttcggacaaactggatcgcgcccctcggtccccttgtcgacacatttgaagaaaagcttgccgcctatgcggggacgtccggagccgcggcagtcagctcaggaacagctgcgatccacctggccttgaaattgctcggcgtcggcaaaggcgatacggtcttctgctcttcttttacgtttgtagcgagcgccaatccgatcatatatgagcaggctgaaccggttttcattgattctgaacgggatacatggaacatgtcgcccgaggcgcttgaacgggcgcttgacgaagcggagcgggccaggaatctcccgaaagccgtcatcgtcgtcaacttgtacggccaaagcgcgaaaatggacgagattatggccatttgcgatcgatttgccgtgcctgtcattgaagatgcagccgaatcgctcggttctgtttataaaggcagaaaaagcgggaccttcggacgcttcggcatttattcgttcaacggtaacaaaatcatcaccacatcgggcggaggaatgctggtcagcgatgatgaagacgcgttgaagaaggcgcgctttttagccactcaggcgcgcgagccagccattcattatcagcacgaaaaagcgggctacaattaccggatgagcaatgttctggccggaatcggcatcgcacagctcgccgttctggatgaccgggtacatgccagacgggcggttttcgagcgctataaggaggcgctttccggtatcgaaggtatagaattcatgcctgaggccggcatgtcaaaccgctggctcacgacattaacgttagacacagcaaagattcaaacaacaccggcggacatcatcgaacagctcgcaaatgaaaacattgaggcccgcccgttatggaagcctttgcacagacagcccctttttaaaggcgcggccttttatccgcacgatgaccagggctctgtctgctgcgacttatttcagcgcgggctctgcctgccgtcaggatcaagtatgacgcgaaaagagcaggaccgggtaattcaaatcgttgccgaccggattaaatataaatga

SEQ ID NO: 42 - DNA, 1167 bp, Bacillus subtilis
atgcataaaaaaatctacttatctccccctcatatgagcggcagggagcagcactatatttcagaagcctttcgctcaaactggattgcgccgcttgggccgctcgtgaattcatttgaagaacagctggcagaacgagtcggtgtaaaagcagcagctgcggtcggctcaggaacggcggcgattcatctcgcgctgcgtttgcttgaggtaaaagaaggtgacagcgtgttttgccagtccttcacatttgtagcaaccgccaatccgattctatatgaaaaagcggtgcccgtctttattgattctgagcctgatacgtggaatatgtcgccgacagcccttgaaagagcattggaggaagcgaaaagaaacggaacactgccaaaagcggtaattgccgtcaatctatatgggcagagcgcgaaaatggatgaaatcgtaagcctgtgtgatgcatacggagttcctgtcattgaggacgcagccgaatctctcggcacagtctataaagggaagcaaagcggaacattcgggcgcttcggcattttttcatttaacgggaacaaaatcatcaccacatcagggggagggatgctcgtttcaaatgatgaagccgcaattgaaaaagcaagatttctcgcttcgcaggcacgagagccggctgttcattatcagcacagtgaaattggacataattacaggctgagcaatatcttagccggcgtcggcattgcccagcttgaggtgctggatgagcgagtggagaaaagaaggaccattttcacgagatacaaaaatgcgctcggtcacttagacggcgtccgctttatgccagagtacgcagcaggcgtatccaatcgctggctcaccacgctcacacttgataacgggctcagcccatatgacatagttcaacgtcttgctgaagaaaacattgaagcccgtccgctgtggaagccgctccatacccagccgctgttcgatccggctttattttattctcatgaagatacaggaagcgtatgcgaagatttgttcaagcgaggaatctgtctcccatcggggtccaatatgacagaagatgagcaaggccgggtcattgaagtgctactgcacttattccatactgtcgaggtgaagaaatggacagcaagcattcgatga

SEQ ID NO: 43 - DNA, 966 bp, Bacillus amyloliquefaciens
atggacagccgccatgttatgagccgattaaaagagacgctgacgggtctgctcagcgttataccgccgcaatccgacatcatatatgccgactatcccctctacggaaatgtaggggatttattgattatgaaagggacggaggcgttttttaaagcgcacggcatccgcgtgaaacagcgctggaatcccgacaattttccattcgggcgccgggcggacaaaaagacgatcatcgtctgtcagggtggcggaaatttcggggatctctatccgtactatcagacgttcagggaaaaaatcgtcaaatcctttccggaaaacaggatcgtcatcctgccgcaatcgatttattatcaggatgagacacggctgcaaaagacatcggcgctttttgcggagcacaaggatctccacctgtttacgagagatcatgtgtcatacgagactgcgaaacgctttttttcggcgaatcatatcaggcttatgcctgatatggcccatcagctgtatccgatcgcggcgtcagcggttccgtcacgcggcaggctttattttatccgcacggatggggaaaacaacccgaagctgcaaaacaattcttccgttaaaaactgtgactggcaggatgttctgtcagcaagcgaccgcaggggaatcgcttttttccaaacgctgaatgttttgaataaaaaagcaggaaacccgctgccgattgcgcggttttggaaacgctattccgattacttaacgaaaaaagcggtcctgtttttcagcaggtacgaatcggtggaaacgtcaaggctccacggccacatcctgtcctcgcttctcgggaaaccgaataccgtcattgataattcatacgggaaaaacgcgaactattattacacctggacgcacgaagcgccggacgtccggctgatcggtgaaaccgccggcacgaaggaaaaccttccgctttga

SEQ ID NO: 44 - DNA, 978 bp, Bacillus licheniformis
atggcgattacatattccatggacagcttaaagcataagctggcagaaattttggatgtcattccaaggcattcatcagtcgtttacttggactacccgctatacggaaacgtcggggatctattgatcatgaaaggaacggaagctttttttgaagcatacggcatcaaggtgcgcgaaagatggaatgcggagaatttcattccgggccgccgcattccaaaggacgccatcattgtttgtcaggggggcggcaatttcggcgacttgtaccctcacttccagcagttcagagaacgggtggtcgaacattacccggacaaccggatcgtcattctgccgcagtcgatttattatgagcatgaagaaaatataataaaaacgcgcggcattttggcggctcacccggatctgcacttattcacgcgggaaaaggcatcattcgattttgccgtcaagcgtttcgaagaggtgaaaaacatcaaaatgatgcccgatatggctcaccagctctggccgatcgcggcacctgccgaaaagccgtccgagtccgttctgcggttgatcaggaccgacaaagaagccaacagcagcctgcagaaagcaggggagccggacacgtacgattggaacgtcatcttatcagaaggcgacaaacgcggaatcaaacgcctgcagacgatcaatgtcctcaacaaaaaagcgggcaatccgctgccgatcgcttcttattggaaacgcttttcggacagcctcgtcgacaaatcaatccgtttcttcagccgctatgaatcagtcgttacttcaaggctgcacggccacatcttatcgtgcctgctcggaaaagaaaacgtagtgattgacaactcctacggaaaaaacgcgaattactacaatacatggatgaaagacatcccgaatacgaaactgattcaaaaccatcagacagaagcagaaaaaccgcctgttcacgtatga

SEQ ID NO: 45 - DNA, 969 bp, Bacillus subtilis
atggacagcaagcattcgatgatcagcctgaaacagaaactgtccgggctgctcgacgtcattccgaaacaatcagagattatatatgctgactatcctctatatgggaatgtaggggatttatttattatgaaaggaacagaagccttctttaaagaacacgggattcgggtcagaaaacgctggaatccagacaatttcccaatcgggcgaaagcttgatccgaatctcatcatcgtctgccagggaggcggaaacttcggggatctgtatccgtattatcaaggctttagagagaaaatcgtccaaacctatccgaaccacaaaattgtgatcctgccgcaatcgatttattttcaaaacaaagacaacctcaagcggacggcagagatattttctaagcatgcgaaccttcacatcatgacaagggaaaaagcctcctatgctacggcacaggcctattttacaacaaatcacattcagcttctgcctgatatggctcatcagctgtttcccgtcattcccacgcagcagccgtccaatcaaaagctgagatttatccgaacagatcatgaagcaaaccaggcgcttcaggaacacgcagaagcggaaagctacgactggcgcacggtgctgtcagcttcagaccgccggacgattgcttttctccaaacgctgaacgtcttgaataaaaaagcaggcaaccctttgcccattgcgtatatatgggaaaaatactcggattatatcgtccaaaaagcgattcggtttttcagccgttacgaatcggtggaaacatcaaggctgcacggccacatcctgtcttctcttcttcaaaaagaaaacacggtcattgataattcctacgggaaaaacgccaattactttcatacctggatggaaggcgtgccaagcacccgtctcatccagcacgcctcaaagaaggaaaaccttcctgctcacatgtga

SEQ ID NO: 46 - protein, 235 aa, Bacillus amyloliquefaciens
MNENMSFKELFDIIKHRFLLIFIMTAVVTLVTGYIQFRVISPVYQASTQVLIHETSGEKNSNLSDVQLNLHYNNTFQTIMKSPVVLEKVKQKLHLSETASALKAKITTSSETDSEIINAAVQDENPKQAAAIANTLMKTFKKEVRDRMNIKGVIVLSEAKASESPMVKPSRIRNIMMAFGAALMAGVTLAFFLHFLDETVKSERQLSEKTDLPVLGVVYDIKNQQTRSDEKHFGE

SEQ ID NO: 47 - protein, 250 aa, Bacillus licheniformis
MKENIDFRELIAILRKRTVLILVLTIGVTLTTGIIQFYVLTPVYQASTQILVHQVGEKKGSATYSDIQINLQYTRTFQALLKNPVILEQVKRELDLPYSAGRLGEKIATSSESESEIINISVQDENQKRAADIANTLTAVLKKEIKQIMNTDRVTVLSKAEIVDSPTPVRPNYKMNILLAFGAALMTGIALAFFLDFIDDTVARPSQVEKEAGFIYLGSIEQMKHKKSLFRGDPDMNIRVKAGRSEPLGY

SEQ ID NO: 48 - protein, 234 aa, Bacillus subtilis
MNENMSFKELYAIVRHRFVLILLITIGVTLIMGFVQFKVISPTYQASTQVLVHESDGEENSNLSDIQRNLQYSSTFQSIMKSTALMEEVKAELHLSESASSLKGKVVTSSENESEIINVAVQDHDPAKAAEIANTLVNKFEKEVDERMNVQGVHILSEAKASESPMIKPARLRNMVMAFGAAVMGGITLAFFLHFLDDTCKSARQLSERTGLPCLGSVPDVHKGRNRGIKHFGE

SEQ ID NO: 49 - protein, 226 aa, Bacillus amyloliquefaciens
MGFRKKKSRRGLAQISVLHHKSLVAEQYRTIRTNIEFSSVQTHLRSILVTSSVPGEGKSFSAANLAAVFAQQEKKVLLVDADLRKPTIHETYQLENVQGLTNVLVGNASLGETVQKTLIDNLYVLTSGPTPPNPAELLSSKAMGELIQEMYSRYSLVIFDSPPLLAVADGQVLANQTDGSVLVVLSGKTKMDTVQKAKDALQQSKAKLLGALLNKKKIKKTEHYSY

SEQ ID NO: 50 - protein, 233 aa, Bacillus licheniformis
MSRLGIRKKRSRKYQSALVALHQPNTPIVEQYRTIRTNIEFSSFEKPFKSLLITSGLPGEGKSFSASNLAIVFSQQEKKVLLIDADLRKPTIHKIFELDNHSGVTNVLMKKSTLENVVQQSQAENLHVLTSGPIPPNPSELLSSQAMEDLLAEAYDQYDLVILDSPPLLPVADAQILANQVDGSILVILSGKTKLDNAIKSRDALNSSKSELLGAVLNGRKVKKARQYNYATM

SEQ ID NO: 51 - protein, 227 aa, Bacillus subtilis
MFFRKKKARRGLAQISVLHNKSIVAEQYRTIRTNIEFSSVQTNLRSILVTSSVPGEGKSFSAANLAAVFAQQQEKKVLLVDADLRKPTINQTFQVENVTGLTNVLVGNASLSETVQKTPIDNLYVLTSGPTPPNPAELLSSKAMGDLISDIYEQFSLVIFDSPPLLAVADAQILANQTDGSVLVVLSGKTKTDTVLKAKDALEQSNAKLLGALLNKKKLKKSEHYSY

SEQ ID NO: 52 - protein, 597 aa, Bacillus amyloliquefaciens
MIFALDTYLVLLSVVIGYQFFEDSYHFYDSGALLLTAVSMLISHHVCAFMFHQYKQVWTYTGIGELLALLKGITLSAAVTAAVQYGVFHTILFRLLAVSWMVQLLFIGGSRMISRVLKETIGKKQNDSSRALIIGAGAGGTLLARQLTQKNDLGIMPVAFIDDDQTKHKLEIMGLPVIGGKESILPAVQRLRIHHIIIAIPSLRTHELQTLYKECVQTGAHIKIMPQFDEILLGTQAAGHIRDVNAEDLLGRKPVTLDTSKISDSIKGKTILVTGAGGSIGSEICRQISSFRPREIVLLGHGENSIYSVHGELSARFGKEVLFHAEIADIQDRDKIFTLMKKYEPHVVYHAAAHKHVPLMEHNPEEAVKNNILGTKNVAEAADMCGTETFVLISSDKAVNPANVMGATKRFAEMVIMNLGKISRTKFAAVRFGNVLGSRGSVIPIFKKQIAKGGPVTVTHPAMTRYFMTIPEASRLVIQAGALAKGRQIFVLDMGEPVKIVDLAKNLIHLSGYTTEQIPIEFSGIRPGEKMYEELLNHNEVHTEQIFPKIHIGKAVDGDWAVLIRFMEEFSRLPEEELRKRLFEAIESVHEEAAAGV

SEQ ID NO: 53 - protein, 605 aa, Bacillus licheniformis
MTYRRRLSIITALDSYLVLLSIFIGYQLILPSYDLYPSEMLLMTSLILLGAQHLFAHCFHLYKKVWEYASIGELYVLLKSITLSHLVTAALELFFFQNVPVRLLCLSWLFQLILIGGSRMMWRIIREQVNKESKGSLRALIIGAGSAGSLIAKQLVQKPELNIKPVAFIDDDKTKYRLEIMGLPVLGGKEQIMQAVRQWNIDRIIIAIPSLSVTQMQEMYKACAQTGVKTQIMPKIDEILLGRHPVGQLRDVKAEDLLGREPVQLDTSEISNTVKDRVVLVTGAGGSIGSEICRQISKFKPKSIILVGHGENSIHSILLELKEKFGKHVAYYPEIADIQDREKMFLLMERYKPNVIYHAAAHKHVPLMEKCPKEAVKNNILGTKNVAEAADETEVETFVLISSDKAVNPANIMGATKRFAEMLIMNLGKTSKTKFVAVRFGNVLGSRGSVIPIFKKQIAKGGPVTVTHQDMTRYFMTIPEASRLVIQAGALAKGRQIFVLDMGEPVKIVDLAKNLIQLSGYTTEQIKIEFTGIRPGEKMYEELLNQNEVLAEQVFPKIHIGKAVDVEWTVLKSFMDEFMYLSDRELRERLFKAIGQHEKKLVTAH

SEQ ID NO: 54 - protein, 598 aa, Bacillus subtilis
MIIALDTYLVLNSVIAGYQFLKDSYQFYDSGALLLTAVSLLLSYHVCAFLFNQYKQVWTYTGLGELIVLLKGITLSAAVTGVIQYAVYHTMFFRLLTACWVLQLLSIGGTRILSRVLNESIRKKRCASSRALIIGAGSGGTLMVRQLLSKDEPDIIPVAFIDDDQTKHKLEIMGLPVIGGKESIMPAVQKLKINYIIIAIPSLRTHELQVLYKECVRTGVSIKIMPHFDEMLLGTRTAGQIRDVKAEDLLGRKPVTLDTSEISNRIKGKTVLVTGAGGSIGSEICRQISAFQPKEIILLGHGENSIHSIYTELNGRFGKHIVFHTEIADVQDRDKMFTLMKKYEPHVVYHAAAHKHVPLMEHNPEEAVKNNIIGTKNVAEAADMSGTETFVLISSDKAVNPANVMGATKRFAEMIIMNLGKVSRTKFVAVRFGNVLGSRGSVIPIFKKQIEKGGPVTVTHPAMTRYFMTIPEASRLVIQAGALAKGRQIFVLDMGEPVKIVDLAKNLIHLSGYTTEQVPIEFTGIRPGEKMYEELLNKNEVHAEQIFPKIHIGKAVDGDWPVLMRFIEDFHELPEADLRARLFAAINTSEEMTAASVH

SEQ ID NO: 55 - protein, 379 aa, Bacillus amyloliquefaciens
MTKKVLFCATVDYHFKAFHLPYFQWFQDMGWEVHVAAGGNMNLPFVDEKFSIPIRRSPFHPENLSVYRRLKRLIQDNGYDMIHCHTPVGGVLARLAARQARQKGTKVLYTAHGFHFCDGAPLKNWLLYYPIEKFLSSYTDCLITINEEDYERAKQMKKTACGAKKIHGIGVNTDRFRPVSRAESERLREKHGFGAGEFILIYPAELNGNKNQGLLIEAAALLKNRIPELKLVFAGEGAMEEPYRKKAESLGVSDIVRFYGFCRDIHELIQLADLSVASSIREGLGMNLLEGMAAEKPAVAADNRGHREIIEDGVNGFLVPAGDSAAFADRIEKLYRSPGLRKAMGQEGRRTAECFSETRTVKEMAHIYAGYMDKKEKSL

SEQ ID NO: 56 - protein, 382 aa, Bacillus licheniformis
MTRTVLFCATVDYHFKAFHLPYLKWFKEQGWNVHIAAKGDMTLPYTDKKFDIDIRRSPLNASNIAAYRELARIIDEHRYSIIHCHTPMGGVLARLAARKQRKEGTKVIYTAHGFHFCQGAPLKNWLLYYPIEKGLSALTDCLITINEEDFVLAKGLRKALRTEKIHGIGVDTERFHPVSETEKMLLRKTYGFKEDDFILIYPAELNANKNQALLIETAAALKDRAPNLKVVFAGKGQMEQKYRNHAEQKGVSSLVMFAGFQKNIHEWIQLADVSVASSIREGLGMNLLEGMASGKPAVAADNRGHREVIQEGVNGFLVPQGDAGTFSDRILQLYRLPSLRKKMGDAGRRTAAAFSQQRTVKEMAGIYSSFMDNETVERRLKG

SEQ ID NO: 57 - protein, 381 aa, Bacillus subtilis
MTKKILFCATVDYHFKAFHLPYFKWFKQMGWEVHVAANGQTKLPYVDEKFSIPIRRSPFDPQNLAVYRQLKKVIDTYEYDIVHCHTPVGGVLARLAARQARRHGTKVLYTAHGFHFCKGAPMKNWLLYYPVEKWLSAYTDCLITINEEDYIRAKGLQRPGGRTQKIHGIGVNTERFRPVSPIEQQRLREKHGFREDDFILVYPAELNLNKNQKQLIEAAALLKEKIPSLRLVFAGEGAMEHTYQTLAEKLGASAHVCFYGFCSDIHELIQLADVSVASSIREGLGMNVLEGMAAEQPAIATDNRGHREIIRDGENGFLIKIGDSAAFARRIEQLYHKPELCRKLGQEGRKTALRFSEARTVEEMADIYSAYMDMDTKEKSV

SEQ ID NO: 58 - protein, 280 aa, Bacillus amyloliquefaciens
MNAGAQPKISVIMGIYNCEETLAESIESILSQSYKNWELIMCDDASTDGTYQIARRYADHYSDRITLIQNKTNQRLAASLNRCLTYATGDYIARQDGDDISNPRRLEKQAAFLNKHAHYQVVGTGMLVFDEFGVRGARLLPPVPKPGIMAKGTPFCHGTIMMRAEAYKALGGYRSVRRTRRMEDIDLWLRFFEAGFRGYNLQETLYKVREDSDAFKRRSFTYSIDNAVLVFQACRRLKLPISHYAYIAKPLIRAITPPAVMNRYHKNRDIRQKEGLAEHD

SEQ ID NO: 59 - protein, 280 aa, Bacillus licheniformis
MIGGQKPKVSVIMGVYNCENTIAESIESILNQTYKNWELIICDDASTDGTYAVARRYADHYADKIKLIKNEKNQRLAASLNHCLQYAGGKYIARQDGDDISLPRRFEKQVAFLESQSHYHVVGSGMMAFDENGIRGVRMLPSSPEPRIMAKGTPFCHATIMMRADVYEALDGYRVGRRTRRMEDVDLWLRFFEAGFTGYNLQEALYKVREDESAFKRRKLSYSIDNAFIVFAACRRLKLPLSDYIYTMKPIIRGLMPPFIMNRYHKRRLMNEGGGVVKHE

SEQ ID NO: 60 - protein, 278 aa, Bacillus subtilis
MNSGPKVSVIMGIYNCERTLAESIESILSQSYKNWELILCDDASTDGTLRIAKQYAAHYSDRIKLIQNKTNKRLAASLNHCLSHATGDYIARQDGDDLSFPRRLEKQVAFLEKHRHYQVVGTGMLVFDEFGVRGARILPSVPEPGIMAKGTPFCHGTIMMRASAYRTLKGYRSVRRTRRMEDIDLWLRFFEEGFRGYNLQEALYKVREDSDAFKRRSFTYSIDNAILVYQACRRLKLPLSDYIYIAKPLIRAFMPAAVMNRYHKKRVMNQKEGLVKHE

SEQ ID NO: 61 - protein, 378 aa, Bacillus amyloliquefaciens
MTDKPMRVLHIFSGMNRGGAETMMMNLYRKMDRTKVQFDFLTHRNDPCAYDEEILALGGRLFYVPSIGSTNPITFVKQVKRVIQEKGPFAAVHAHTDFQSGFIALAARLAGVPVRICHSHSTSWRGRASRLAGMQLFVFRRLITANATALCACGKDAGRFLFGKEKDVHLLPNGIDLGLFAGGGADTEAEKRKRGIADGRLVIGHIGRFTEEKNHEFLLRLAADMKERGIGLQLILAGDGPLRTDMENLAAKLGLDDDVRFIGIEDRVHALLKTLDVFVMPSLYEGLPVTLVEAQASGVPCVISDGITEEADAGLGLVKRLSLKEPPGQWASAVLRAAEAAKPDGERIKETLRRQGYDAGENAGAVMKLYNMNWKKEQ

SEQ ID NO: 62 - protein, 386 aa, Bacillus licheniformis
MNDGSVRPKRVLHIVSGMNRGGAETMIMNIYRHTDRRHIQFDFISHREETCDYDPEIITRGGRVFYVPSIGRSGPVAYIKNIRRILVEKGPYAAVHAHTDFQTGFAALAARLAGVPVRVCHSHNTAWKPNPRFWDTWQLLAFRRLIFSSATALCACGKDAGRFLFGAKKMGENAVHLLQNGIELDRFKEANGVSKTNAKKSFGIKEDALVIGHVGRFFEQKNHAFLLGLAAYCKKSGIPFQAVFAGDGPLRRQMEEKAAALGVKDDILFLGVVEDIPALMQAFDVFVMPSLFEGLPLVLVEAQASGLPCIVSDNITEETDLGLGLLQRLSLNAGFERWAEDISRAAQPKKPAWPEIERSLAERGYDAKANLARLMDIYSISAAEGQ

SEQ ID NO: 63 - protein, 384 aa, Bacillus subtilis
MNSSQKRVLHVLSGMNRGGAETMVMNLYRKMDKSKVQFDFLTYRNDPCAYDEEILSLGGRLFYVPSIGQSNPLTFVRNVRNAIKENGPFSAVHAHTDFQTGFIALAARLAGVPVRVCHSHNTSWKTGFNWKDRLQLLVFRRLILANATALCACGEDAGRFLFGQSNMERERVHLLPNGIDLELFAPNGQAADEEKAARGIAADRLIIGHVARFHEVKNHAFLLKLAAHLKERGIRFQLVLAGDGPLCGEIEEEARQQNLLSDVLFLGTEERIHELMRTFDVFVMPSLYEGLPVVLVEAQASGLPCIISDSITEKVDAGLGLVTRLSLSEPISVWAETIARAAAAGRPKREFIKETLAQLGYDAQQNVGALLNVYNISTEKDHNR

SEQ ID NO: 64 - protein, 367 aa, Bacillus amyloliquefaciens
MIVYVVNMGIVYIWSWFAKMCGGRDQSLSTGYRPNQLVMIVPLLSLVLVSGLRYRVGTDFQTYALMYKLAGNYGSIWEIFGFGAKKAAVDPGFTALIWLMNFITKDPQIMYFTVAVVTCSFILKGLVEFGRPFELSVFLFLGTYHYYASFNGIRQYMVAAVLFWAVRYVISGSWKRYMSIVLLCSLFHSSALIMIPVYFLVRKRAWSPAIFGLSALFLLMTFLYQKFISLFVVVLENSSYGHYEKWLMTNTNGMNVVKIAVLLLPLFLAFCYRERLRKLWPDSDVIVNFCLLGLLFGLLATKDVIFARFNIYFGLYQMILIPYFVRIFDEKSNALIYIAIIVCYFLYSYLLMPVDSSVLPYRTIFSR

SEQ ID NO: 65 - protein, 367 aa, Bacillus licheniformis
MAVYMLNMGIVFVWSWFAKMYGREDHRLPTGYRPNAILTVVPLASLIIVAGLRYKVGTDYHTYMLLYELAGKYNSIWEIFGFGTGKSSTDPGFTALLWILNQISADPALMFAVVAAITYIYIVKTLYVYGRPFELSMFLFIGMFHYYASFNGIRQYMAAAILFWAVRYLIDGKLVRYMIVVLICSLFHSSALIMIPVYFIVRRKAWSPVLWCLMLVFLAGTFLYQKFLSVFLVVLENSQYGHYEEWLMKNTNGMNVIKIIVLLLPLALAFCFREQLRKRWPEVDYIVNLCLIGFLFGILATKDVIFARFNIYFGLYQLILVPYFVRIFEPKSNALLYVLILICYFLYSFMLMPFDSSVLPYRTIFER

SEQ ID NO: 66 - protein, 367 aa, Bacillus subtilis
MIVYAVNMGIVFIWSWFAKMCGGRDDSLATGYRPNKLLIWIPLASLVLVSGLRYRVGTDFQTYTLLYELAGDYQNVWQIFGFGTAKTATDPGFTALLWLMNFITEDPQIMYFTVAVVTYSFIMKTLADYGRPFELSVFLFLGTFHYYASFNGIRQYMVAAVLFWAIRYIISGNWKRYFLIVLVSSLFHSSALIMIPVYFIVRRKAWSPAIFGLSALFLGMTFLYQKFISVFVVVLENSSYSHYEKWLMTNTNGMNVIKIAVLVLPLFLAFCYKERLRSLWPQIDIVVNLCLLGFLFGLLATKDVIFARFNIYFGLYQMILVPYFVRIFDEKSNALIYIAIVVCYFLYSYLLMPVDSSVLPYRTIFSR

SEQ ID NO: 67 - protein, 345 aa, Bacillus amyloliquefaciens
METPAVSLLIAVYNTETYLERCLDSLLNQSLANIEIVAVNDGSTDQSPAVLEAYQKRDERIRVIHQQNRGLGAVRNKGIEAARGEFIAFIDADDWVEPDYCLRMYEKAKADQADLVICEYAAEFADTGKTAVSTIASAYAGRPKQLYLKDLFEGRVSGFSWNKLYKRTMIEHHQLRFPLRDELEHVEDQYFSLRAHVYAGAVSYVDQPLYHYRIHLTSIVQSYQKKLFDSGLVLYRLNETFLRENGCLQEYRQELDFFIVQHGTVCLLNEWKRNNGGRFSEKWKNISRICADPVFRLSLSKTGTAPFDAKRSCLLLLAKLKLIPFVSLASAAYQRAIEYKMKIRG

SEQ ID NO: 68 - protein, 343 aa, Bacillus licheniformis
MENPAVSLLVAVYNTEAFLPNCLQSLISQTLKNIEIIIVNDGSTDGSQKIIDHYARKDGRIKTIQQDNQGLGAVRNKGIEAASGEYLAFIDSDDWIEPDYCQSMYEKAKDEDADLVICDYAVEIQDTEKTVCPDIGKNYEGKPKEAFMKDLLKGKVSGFSWNKLYRRSLIERHKLVFPLRDELENIEDQYFSFRCLLYANTAAFVTKPLYHYRVHLASIVQKYQAGLFEDGLALYEANLDCLTKHGELPALKEALHVFIVNHGCISILNECKSRNKNPSIEKYKNIRSICACPEFRGKISAVDMSAFDSKKKLLLMLIRLRLMPAVYGFAAIYQKMIEHRMKK

SEQ ID NO: 69 - protein, 344 aa, Bacillus subtilis
METPAVSLLVAVYNTETYIRTCLESLRNQTMDNIEIIIVNDGSADASPDIAEEYAKMDNRFKVIHQENQGLGAVRNKGIEAARGEFIAFIDSDDWIEPDYCEQMLRTAGDETDLVICNYAAEFEDTGKTMDSDIAQTYQDQPKEHYIKALFEGKVRGFSWNKLYRRSMIEAHRLSFPLRGELEHVEDQFFSFRAHFFARSVSYVKTPLYHYRIHLSSIVQRYQKKLFESGLALYETNAAFLQENNKLEEYRKELDTFIVLHSSICMLNEWKTSGSRRLFEKLRNVGVICADPVFQESLSKTGTAPFDAKRSCLLLMAKYRMIPFVAMASAVYQRVIEYKMRNRG

SEQ ID NO: 70 - protein, 358 aa, Bacillus amyloliquefaciens
MSIQSLKINLAEWLLLKVKYPSQFHFGTAADGAELTAARKKIILTLLPSHDNLGDHAIAYASKTFLEREYPDFDIVEVDMKDIYRSAKALIKKRHPDDMVFIIGGGNMGDLYRYEEWTRRFVIKTFRQYRIVQLPATAHFSESKKGRKELKRARKVYNAHPDLLLMARDETTYQWMKRHFPGKTVLKQPDMVLYLDKSERGIPREGIYLCLREDKESALTAEDRTMVKEALAKEYGELYSFTTTVGRRVSRHTREKELEALWNTLKGAEAVVTDRLHGMIFCALTKTPCVVIRSFDHKVMEGYQWLKNIPNMTLLERPDPEAVTAAVNRLLSGKHEEGGSLRSVYFAGLRSKISGDAQ

SEQ ID NO: 71 - protein, 359 aa, Bacillus licheniformis
MTFQELKINLAEWLLLKVKYPSEYVMGTPGLRRFEQYKGKKKIILTLIPSHDNLGDHAIALASRTFIENEFPDFELIEIGINDIYKHAKALMRIRHPEDMVFIIGGGNMGDLYRNEEWTRRFIIKTFKHYKIVQLPATAHFSETLRGKKELKRAKKIYNSHRRLFMMARDDTTYQFMKQHFSNQTIVKQPDMVLYLKKEQQSEREGVLVCLREDKESFLRPEERKKLLKAVGDEYGGAKTFTTTIGRRVSRVSREKELNRLWDQLRGAEVVVTDRLHGMIFCAITGTPCVVIRSFDHKVLEGFRWLKDVPSMKLVENPDAAEVLGAIEELVKTGDSHRETPARDHYFADLRRKIMGDVQ

SEQ ID NO: 72 - protein, 358 aa, Bacillus subtilis
MSLQSLKINFAEWLLLKVKYPSQYWLGAADQPVKAAAHQKKIILTLLPSHDNLGDHAIAYASKAFLEQEYPDFDIVEVDMKDIYKSAKSLIRSRHPEDMVFIIGGGNMGDLYRYEEWTRRFIIKTFHDYRVVQLPATAHFSDTKKGRKELKRAQKIYNAHPGLLLMARDETTYQFMKQHFQEKTILKQPDMVLYLDRSKAPAEREGVYMCLREDQESVLQEEQRNRVKAALCEEFGEIKSFTTTIGRRVSRDTREHELEALWSKLQSAEAVVTDRLHGMIFCALTGTPCVVIRSFDHKVMEGYQWLKDIPFMKLIEHPEPERVTAAVNELLTKETSRAGFPRDVYFKGLRDKISGEAQ

SEQ ID NO: 73 - protein, 344 aa, Bacillus amyloliquefaciens
MNAPLVSVIVPMYKTEPFIKACAVSLTKQMLRDIEIIFVNDGSPDQSGRMAEQFAAEDARIRVIHQENGGLSSARNAGIKAARGRYIGFVDGDDYVTETMFERLYEEAEKNRLDIAGCGYYKETPSKERAYMPPSIPPGRVFTAAEMTDLLTCAHEHRFIWYVWRYIYRREVLQGLLFHEDIRFAEDSPFNLAAFRHAARVKVIDEGLYIYRENPTSLTETPFKPHLDDELQKQYEAKMAFYEANGLTDACQSDINTYLCKHQIPMLIANACAAPQPSHEITAHIGRILTYDMVKTAVRSTPCRHKQLLAGERVVLGLCKMRLPLLLHAFFDRKTKEKGSAEGA

SEQ ID NO: 74 - protein, 339 aa, Bacillus licheniformis
MKPFISIIVPMYNVEDYIEECVDSLRRQTLKNIEIILVDDGSPDRSGEIARTYCSLDARVKVIHKKNGGLSSARNAGLQAATGDYVGFVDGDDFVLPAMFENMYAAAKKDDLDIVMCGYHKHSDTEDAYFPPPLPTDRLLLSWDIKRELKKAHETRFIWYVWRNLYRRDLLKKNQLYFFEDIRFAEDSPFNLYAFYAAKRVRAIDEGYYMYRCNPDSLTEAPFKPYMDESLKRQYRAKRRFYETFQLLDECADDLETYTCKHQIPMLLANACAEPKPSKQVRRHIKDILSYRMVQSCVKATSLRNRNLLIGQRLVLLLCKLNIPILLELFFKRNLPSKG

SEQ ID NO: 75 - protein, 344 aa, Bacillus subtilis
MIPLVSIIVPMYNVEPFIEECIDSLLRQTLSDIEIILVNDGTPDRSGEIAEDYAKRDARIRVIHQANGGLSSARNTGIKAARGTYIGFVDGDDYVSSAMFQRLTEEAEQNQLDIVGCGFYKQSSDRRTYVPPQLEANRVLTKPEMTEQLKHAHETRFIWYVWRYLYRRELFERANLLFDEDIRFAEDSPFNLSAFREAERVKMLDEGLYIYRENPNSLTEIPYKPAMDEHLQKQYQAKIAFYNHYGLAGACKEDLNVYICRHQLPMLLANACASPNSPKDIKKKIRQILSYDMVRQAVRHTPFQHEKLLRGERLVLALCKWRLTFLIKLFFEQRGTMKGSAKQA

SEQ ID NO: 76 - protein, 505 aa, Bacillus amyloliquefaciens
MKFAINFGANVTAFLLSVFLSVWMTPFIVKTLGVEAFGFVHLTQNIINYFSIITVALSSVVVRFFSVAAHRGNRDEANAYVSNYLAASVVISLLLAVPLAGTAFFIDRIMNVPAGLLTDVRLSIVIGSVLFMLTFFMAGFATGPFFANKLYITSSIQAVQMLVRVLCVLALFTCLPPKIWQIQLSALAGAVCAAVLTFFFFKKLIPWFSFSRKTLSLQTSKVLFSAGAWSSVNQIGVLLFLQIDLMTANLVLGPSEAGVYAAIIQFPLLLRSLAGTLASLFAPVLTSYYSKGDMEGLLSYANKAVRMNGLLLALPAALLGGLAEPFLAIWLGPSFVQTAPLLYIHAAYLAVSLSVMPLFYVWTAFNKQKTPAVVTLCLGGLNVILAVVLSGPAHLGLYGITIAGAVSLILKNAVFTPLYVSHITGFQKTAFYKGMFGPLAAAVFAWAVCRGIRLFSPLDGWAGLIAAGLAVCISYAAFAFFFICTKEERRLALQKCRKVKGAVQI

SEQ ID NO: 77 - protein, 515 aa, Bacillus licheniformis
MNKTFVLNLGANMASFLLSVLFSMWLTPYVIKTLGVEAFGFVHLTQNMINYFSIITVALSAVVVRFFSVSAHRGALDEARGYMNTYIVSSLVLSVILFFPLGGTVFFIDQIIRVPAGLLGDVQVALLIGSLLFLLTFVMSGFAAGPFFANKIYITSTIQAIQMLIRVLSVLLIFAWFAPKIWHIQLAALIATASACILSIFFFKRLIPWFTFRVRDMSFAKCKKLLQAGGWSSVSQVGILLFLQIDLMVANVMLGVSESGMYAAIIQFPLLLRTLSGTLAAVFSPTITLYYSKGDKEGLVRYANQAVRFNGILLALPAALLGGLAGPFLSLWLGPSFEHLKWLLLIHAGYLVVSLSPAPLFYIFTAYNKLRTPALTTVAFGVVNLLLAIVLSGPAGLGLYGIALAGAAALTLKNVVFTPIYASKITGERKRVFYKGIYGPVAGASFTLAVCYALQYLFSIVSLLSLFVTALAATLAYGLFAYFVMLTKAERRIVTTKLQAYRCSLSFPFQKGFFK

SEQ ID NO: 78 - protein, 505 aa, Bacillus subtilis
MKFTINFSANLTAFLLSVFLSVWMTPFIVKTLGVEAFGFVHLTQNVINYFSVITVALSSVVVRFFSVAAHRGEREKANAYISNYLAASVLISLLLLLPLAGSAFFIDRVMNVPQALLADVRLSILIGSVLFILTFLMAGFGAAPFYANRLYITSSIQAVQMLIRVLSVLLLFACFAPKIWQIQLAALAGAVIASVLSFYFFKKLIPWFSFRMKDLSFRTSKELFQAGAWSSVNQIGVLLFLQIDLLTANLMLGASASGKYAAIIQFPLLLRSLAGTVASLFAPIMTSYYSKGDMEGLMNYANKAVRLNGLLLALPAALLGGLAGPFLTIWLGPSFSTIAPLLFIHAGYLVVSLAFMPLFYIWTAFNQQKTPAIVTLLLGAVNVVLAVTLSGPAHLGLYGITLAGAISLILKNAIFTPLYVSRITGYKKHVFLKGIIGPLSAAVFAWTVCKAIQFIVKIDSWPSLIATGVTVSFCYAVFAFMLVCTKEERQLVLKRFRKTKGAVNL

SEQ ID NO: 79 - protein, 202 aa, Bacillus amyloliquefaciens
MKVKRVFDIAAATLLLCGASVILLFAMAAVRCAIGSPVLFKQTRPGHNGRPFTLYKLRTMTDARDENGVLLPDHLRLTKTGRLIRKLSIDELPQLFNVLKGDISLVGPRPLLMDYLPLYTAEQARRHEVKPGITGWAQVNGRNAISWEEKFKLDVWYVDNRTFLLDLKILLLTVKKVLVSEGIHQAGHVTAKRFTGSGDMSS

SEQ ID NO: 80 - protein, 200 aa, Bacillus licheniformis
MLAKRFFDLALSVILLVALSPAMILTACLIRWKIGSPVLFRQTRPGLNGEPFTLYKFRTMTDERDAAGNLLSDEKRLTKTGRLIRKTSLDELPQLINVIKGDLSLVGPRPLLMEYIPLYTKRQWRRHEVKPGITGWAQINGRNKVTWEEKFELDVWYVDHRSFLLDLKILLLTVVKVLKSEGVSQDRHVTAEKFTGRRNA

SEQ ID NO: 81 - protein, 202 aa, Bacillus subtilis
MILKRLFDLTAAIFLLCCTSVIILFTIAVVRLKIGSPVFFKQVRPGLHGKPFTLYKFRTMTDERDSKGNLLPDEVRLTKTGRLIRKLSIDELPQLLNVLKGDLSLVGPRPLLMDYLPLYTEKQARRHEVKPGITGWAQINGRNAISWEKKFELDVWYVDNWSFFLDLKILCLTVRKVLVSEGIQQTNHVTAERFTGSGDVSS

SEQ ID NO: 82 - protein, 215 aa, Bacillus amyloliquefaciens
MKKVVLIGNGGHGKVVKEIVKARSDMELAGILDDGFSGFTVRDGLYTGRTKDVHMLRKLVPGAVFTICIGHNGVRKQLAETLGLEHDDYTALIHPGAIVSDTASVGHGTVVMAGAVIQAGADIGAHCIINTGAVADHDNAIGDYVHLSPRAALAGGVKVGEGAHIGIGASVIPRTDIGPWSVIGAGAAVISRIPDHVTAVGVPARVISSIHNEKG

SEQ ID NO: 83 - protein, 208 aa, Bacillus licheniformis
MQNVVIIGAGGHGKVVRELVKERPDTELAGILDDRYAELHVENGLYRGPSAAAEELARLHPDAKFVLAVGQNSIRQQLYERIGLPLDRYAVLIHPSAVVSGSARIQNGAVVMASSVIQADADVGIHAIVNTGAIVEHDNRIGDYVHLSPGTVLTGGVTVMEGAHLGAGTAVIPGKTVGRWSVTGAGAAVIHDIPDNCTAVGVPARMIK

SEQ ID NO: 84 - protein, 216 aa, Bacillus subtilis
MKNVAIVGDGGHGKVIRELINARSDTRLAAVLDDKFKTFEGGKEWYTGPPKAVTELRRLIPDVLFLIAVGNNSVRKQLAERLGLGKDDFITLIHPSAIVSKSAVIGEGTVIMAGAIIQADARIGAHCIINTGAVAEHDNQISDYVHLSPRATLSGAVSVQEGAHVGTGASVIPQIIIGAWSIVGAGSAVIRSIPDRVTAAGAPARIISSIQTSNKG

SEQ ID NO: 85 - protein, 390 aa, Bacillus amyloliquefaciens
MQTNKRIYLSPPHMSGKEQEYIAEAFRSNWIAPLGPLVNSFEARLAEYAGVKSAAAVSSGTAAIHLALRLAGVKKGDVVFCPSFTFVATANPIVYEQAEPVFIDSEWETWNMSPDALERALRDAKRRGRLPKAVIAVNLYGQSAKMDELMSLCDAYGVCLIEDAAESLGSTYKGRQSGTFGRFGIYSFNGNKIITTSGGGMLVSDDEAAIEKARFLASQARDAAVHYQHSELGYNYRLSNILAGVGISQLEVLEDRVRARREIFHRYREALETYPGIRMMPELEGTVSNRWLTALTLDNGVTPEEAVACLAEQNIEARPLWKPLHTQPLFSSSVFYPHSEHERVSENLFSRGICLPSGSDLSSEEQQRVIDALAQLFETKGEKTWTAAML

SEQ ID NO: 86 - protein, 381 aa, Bacillus licheniformis
MSQNKRIYLSPPHMSGDEERYVAEAFRTNWIAPLGPLVDTFEEKLAAYAGTSGAAAVSSGTAAIHLALKLLGVGKGDTVFCSSFTFVASANPIIYEQAEPVFIDSERDTWNMSPEALERALDEAERARNLPKAVIVVNLYGQSAKMDEIMAICDRFAVPVIEDAAESLGSVYKGRKSGTFGRFGIYSFNGNKIITTSGGGMLVSDDEDALKKARFLATQAREPAIHYQHEKAGYNYRMSNVLAGIGIAQLAVLDDRVHARRAVFERYKEALSGIEGIEFMPEAGMSNRWLTTLTLDTAKIQTTPADIIEQLANENIEARPLWKPLHRQPLFKGAAFYPHDDQGSVCCDLFQRGLCLPSGSSMTRKEQDRVIQIVADRIKYK

SEQ ID NO: 87 - protein, 388 aa, Bacillus subtilis
MHKKIYLSPPHMSGREQHYISEAFRSNWIAPLGPLVNSFEEQLAERVGVKAAAAVGSGTAAIHLALRLLEVKEGDSVFCQSFTFVATANPILYEKAVPVFIDSEPDTWNMSPTALERALEEAKRNGTLPKAVIAVNLYGQSAKMDEIVSLCDAYGVPVIEDAAESLGTVYKGKQSGTFGRFGIFSFNGNKIITTSGGGMLVSNDEAAIEKARFLASQAREPAVHYQHSEIGHNYRLSNILAGVGIAQLEVLDERVEKRRTIFTRYKNALGHLDGVRFMPEYAAGVSNRWLTTLTLDNGLSPYDIVQRLAEENIEARPLWKPLHTQPLFDPALFYSHEDTGSVCEDLFKRGICLPSGSNMTEDEQGRVIEVLLHLFHTVEVKKWTASIR

SEQ ID NO: 88 - protein, 321 aa, Bacillus amyloliquefaciens
MDSRHVMSRLKETLTGLLSVIPPQSDIIYADYPLYGNVGDLLIMKGTEAFFKAHGIRVKQRWNPDNFPFGRRADKKTIIVCQGGGNFGDLYPYYQTFREKIVKSFPENRIVILPQSIYYQDETRLQKTSALFAEHKDLHLFTRDHVSYETAKRFFSANHIRLMPDMAHQLYPIAASAVPSRGRLYFIRTDGENNPKLQNNSSVKNCDWQDVLSASDRRGIAFFQTLNVLNKKAGNPLPIARFWKRYSDYLTKKAVLFFSRYESVETSRLHGHILSSLLGKPNTVIDNSYGKNANYYYTWTHEAPDVRLIGETAGTKENLPL

SEQ ID NO: 89 - protein, 325 aa, Bacillus licheniformis
MAITYSMDSLKHKLAEILDVIPRHSSVVYLDYPLYGNVGDLLIMKGTEAFFEAYGIKVRERWNAENFIPGRRIPKDAIIVCQGGGNFGDLYPHFQQFRERVVEHYPDNRIVILPQSIYYEHEENIIKTRGILAAHPDLHLFTREKASFDFAVKRFEEVKNIKMMPDMAHQLWPIAAPAEKPSESVLRLIRTDKEANSSLQKAGEPDTYDWNVILSEGDKRGIKRLQTINVLNKKAGNPLPIASYWKRFSDSLVDKSIRFFSRYESVVTSRLHGHILSCLLGKENVVIDNSYGKNANYYNTWMKDIPNTKLIQNHQTEAEKPPVHV

SEQ ID NO: 90 - protein, 322 aa, Bacillus subtilis
MDSKHSMISLKQKLSGLLDVIPKQSEIIYADYPLYGNVGDLFIMKGTEAFFKEHGIRVRKRWNPDNFPIGRKLDPNLIIVCQGGGNFGDLYPYYQGFREKIVQTYPNHKIVILPQSIYFQNKDNLKRTAEIFSKHANLHIMTREKASYATAQAYFTTNHIQLLPDMAHQLFPVIPTQQPSNQKLRFIRTDHEANQALQEHAEAESYDWRTVLSASDRRTIAFLQTLNVLNKKAGNPLPIAYIWEKYSDYIVQKAIRFFSRYESVETSRLHGHILSSLLQKENTVIDNSYGKNANYFHTWMEGVPSTRLIQHASKKENLPAHM

SEQ ID NO: 91 - DNA, 585 bp, Bacillus amyloliquefaciens
atgcggcttgagcgtctgaactataataagattaaaatctttttaaccctcgacgatctgactgatcggggactgacaaaagaagacctctggaaggactcgtttaaagtccaccagttatttaaagatatgatgaatgaagcaaatacagagctcggctttgaagcgaatggtccgatcgcggtggaagtatattctcttcaggcacaaggcatggttgtgattgtaacgaaaaatcatgacgcggacgcagaagatgacgaatatgacgatgattatatcgagatgcaggtcaaacttgacgaaagcccggatattatttatcagttccattcgtttgaagatatcattcagcttgccggaagccttcaccgaatcgggattaccggcggcaccgtctaccactatgagaaccaatactatgtaagccttgaagacctcggctcccgttccgccgaaggcgtcatcgcggtgctggctgaatacggacacccggcgacgattacgatttacaggctccatgagtacggaaaactcatcatggacggcaatgctgtggagaccatccagaaacatttttcataa

SEQ ID NO: 92 - protein, 194 aa, Bacillus amyloliquefaciens
MRLERLNYNKIKIFLTLDDLTDRGLTKEDLWKDSFKVHQLFKDMMNEANTELGFEANGPIAVEVYSLQAQGMVVIVTKNHDADAEDDEYDDDYIEMQVKLDESPDIIYQFHSFEDIIQLAGSLHRIGITGGTVYHYENQYYVSLEDLGSRSAEGVIAVLAEYGHPATITIYRLHEYGKLIMDGNAVETIQKHFS

SEQ ID NO: 93 - DNA, 639 bp, Bacillus licheniformis
atggaaatcgaaagaataaacgaacacacggttaagttttatatttcctacggtgatatcgaagaccgcgggtttgacagagaagaaatttggtacaatcgagaacgcagtgaagagcttttttgggaaatgatggacgaagtgcacgaagaagaagagtttgccgttgaaggccccctttggattcaagtgcaggcccttgacaagggattggaaatcattgtgacaagagctcagttgtccaaagacggacaaaagctggaactgccgattcctgaagataaaaaacagcatgtagcagaagaaagccttgatgctttgcttgacgactttcaaaaagaagagcaggcagaagagcagaaactgcagtttgttttaaaatttgacgattttgaggatttaatctcactgtcaaaaatgtctgtcagcggttgccaaacgacattgtactctcatgaaaaccgctattatttattcgtggatttcaatgaactgcctgatgaagaggtggaaaaccagctgagcattctgctggaatatgcctcagaatcaaagatgacgattcatatgctgaaagagtatggaaaactgattgcagcggatcatgctcttcatacaataaaaaagcactttgcataa

SEQ ID NO: 94 - protein, 212 aa, Bacillus licheniformis
MEIERINEHTVKFYISYGDIEDRGFDREEIWYNRERSEELFWEMMDEVHEEEEFAVEGPLWIQVQALDKGLEIIVTRAQLSKDGQKLELPIPEDKKQHVAEESLDALLDDFQKEEQAEEQKLQFVLKFDDFEDLISLSKMSVSGCQTTLYSHENRYYLFVDFNELPDEEVENQLSILLEYASESKMTIHMLKEYGKLIAADHALHTIKKHFA

SEQ ID NO: 95 - DNA, 657 bp, Bacillus subtilis
atggaaattgaaagaattaacgagcatacagtaaaattttatatgtcttacggagatattgaagatcgcggttttgacagagaagaaatttggtataaccgtgagcgcagtgaagaacttttctgggaagtcatggatgaagttcatgaagaagaggaattcgctgtggaaggcccactttggattcaagttcaggcactggacaaaggattagaaatcatcgtcacaaaagcccagctttccaaagacggacaaaagctcgaactgccgattcctgaggataaaaagcaagaaccagcatctgaggatcttgacgccttgctggatgatttccagaaagaagagcaagccgtcaatcaggaagagaaggagcaaaagcttcaatttgtcttgcgatttggcgattttgaggatgttatttctctatctaaattgaacgttaacggaagcaaaacgactttatattcgtttgagaaccgatattatttatatgtagatttttgcaatatgactgatgaagaggttgaaaatcagctaagcatcctgctggagtacgcaactgaatcctcaatcagcatacaccgtcttgaagagtacggcaagctgattatttcagagcatgctctagaaacgataaaaaaacactttgcatcatag

SEQ ID NO: 96 - protein, 218 aa, Bacillus subtilis
MEIERINEHTVKFYMSYGDIEDRGFDREEIWYNRERSEELFWEVMDEVHEEEEFAVEGPLWIQVQALDKGLEIIVTKAQLSKDGQKLELPIPEDKKQEPASEDLDALLDDFQKEEQAVNQEEKEQKLQFVLRFGDFEDVISLSKLNVNGSKTTLYSFENRYYLYVDFCNMTDEEVENQLSILLEYATESSISIHRLEEYGKLIISEHALETIKKHFAS

SEQ ID NO: 97 - DNA, 35 bp, Bacillus amyloliquefaciens
gatcggatccatcgccgtccgcaaaaccgatataa

SEQ ID NO: 98 - DNA, 40 bp, Bacillus amyloliquefaciens
cggaagcatttgggagatctcgatcgcttcagcgtacgcg

SEQ ID NO: 99 - DNA, 40 bp, Bacillus amyloliquefaciens
cgcgtacgctgaagcgatcgagatctcccaaatgcttccg

SEQ ID NO: 100 - DNA, 48 bp, Bacillus amyloliquefaciens
gatcggatccattatatcgccaggcaggacggtgatgacatctccaac

SEQ ID NO: 101 - DNA, 28 bp, Bacillus amyloliquefaciens
gatcctcgagaaaacggtaaaagagacg

SEQ ID NO: 102 - DNA, 46 bp, Bacillus amyloliquefaciens
ccattggcgggcttcctccttttctgctccgctccccccttctgtt

SEQ ID NO: 103 - DNA, 46 bp, Bacillus amyloliquefaciens
aacagaaggggggagcggagcagaaaaggaggaagcccgccaatgg

SEQ ID NO: 104 - DNA, 24 bp, Bacillus amyloliquefaciens
gatcctcgagtgaaagaaggcagg

SEQ ID NO: 105 - DNA, 21 bp, Bacillus licheniformis
gaattccattaatagctgctg

SEQ ID NO: 106 - DNA, 36 bp, Bacillus licheniformis
tccatactctttcagcatggtcttcgatatcaccgt

SEQ ID NO: 107 - DNA, 36 bp, Bacillus licheniformis
acggtgatatcgaagaccatgctgaaagagtatgga

SEQ ID NO: 108 - DNA, 23 bp, Bacillus licheniformis
ctcgagcgcatcctcccaaaatc